(19) United States
(12) Patent Application Publication
(10) Pub. No.: US 2007/0083941 A1
(43) Pub. Date: Apr. 12, 2007

(54) Rat TRPM7 polynucleotide and encoded protein

(75) Inventors: Lan He, Plainsboro, NJ (US); Kodangattil Sreekumar, Plainsboro, NJ (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Wyeth, Madison, NJ (US)

(21) Appl. No.: 11/542,939

(22) Filed: Oct. 3, 2006

Related U.S. Application Data
(60) Provisional application No. 60/724,069, filed on Oct. 6, 2005.

(51) Int. Cl. G01N 33/567; C07H 21/04; C12P 21/06; C12N 5/06; C07K 14/705; A01K 67/027

(52) U.S. Cl. 800/14; 435/69.1; 435/320.1; 435/353; 530/350; 536/23.5; 435/7.2

(57) ABSTRACT

This invention relates to isolated nucleic acid and amino acid sequences of rat TRPM7 gene, expression cassettes nucleic acid sequences of rat TRPM7 gene, recombinant host cells comprising nucleic acid and amino acid sequences of rat TRPM7 gene, and methods of screening for modulators of the TRPM7 gene and protein.

### CROSS-REFERENCE TO RELATED APPLICATIONS

This application claims benefit of U.S. Provisional Application No. 60/724,069, filed Oct. 6, 2005, which is incorporated herein by reference in its entirety.

### FIELD OF THE INVENTION

This invention relates to the field of molecular and cellular biology, and pharmaceutical discovery and development. In particular, the invention features isolated nucleic acid and amino acid sequences of rat TRPM7 gene, expression cassettes nucleic acid sequences of rat TRPM7 gene, recombinant host cells comprising nucleic acid and amino acid sequences of rat TRPM7 gene, and methods of screening for modulators of the TRPM7 gene and protein.

### BACKGROUND OF THE RELATED ART

Various publications, including patents, published applications, technical articles and scholarly articles are cited throughout the specification. Each of these cited publications is incorporated by reference herein in its entirety.

The transient receptor potential channel TRPM7 is a member of the TRP superfamily of cation channels that comprises greater than 20 cation channels that play critical roles in varied processes within the body. TRP channels are integral membrane proteins in which the ion-conducting pores are formed by six membrane-spanning helical segments that are similar to those of voltage-gated potassium channels and cyclic nucleotide-gated channels. TRP channels are divided into three families based on their homology. The families are the short TRP channel family, the osm TRP family, and the long TRP family. Long TRP channels can be distinguished by their having particularly long extensions outside the channel segment. Long TRP channels are involved in critical control mechanisms regulating cell growth, differentiation and death (Montell et al., Cell 2002, 108:595-598, Harteneck et al., Trends Neurosci. 2000, 23:159-166).

The TRPM7 channel belongs to the long TRP family. The human TRPM7 protein was first identified by Runnels et al (Science 2001 291:1043-1047) and was identified as a bifunctional protein with kinase and ion channel activities. In another study by Nadler et al. (Nature 2001 411:590-595), TRPM7 was identified as a MGATP regulated cation channel required for cell viability. In an article published by Runnels in 2002 (Runnels et al., Nature Cell Biology, 4, 329-336), TRPM7 is described as a calcium-permeant ion channel. It was also reported that the kinase domain of TRPM7 directly associates with the C2 domain of phospholipase C (PLC) and that 4,5-biphophate (PIP₂), the substrate of PLC, is a key regulator of TRPM7. The TRPM7 channel produces pronounced outward currents at nonphysiological voltages ranging from +50 to +100 mV and small inward currents at negative potentials between −100 to −40 mV when expressed heterologously in mammalian cells (Jiang et al, J. Gen. Physiol. 2005 126(2), 137-150) The basal activity of TRPM7 is reported to be regulated by millimolar levels of intracellular mgATP and Mg²⁺. TRPM7 is activated by depletion of intracellular MgATP and Mg²⁺, and is inhibited by high concentrations of MgATP and Mg²⁺ with an IC₅₀ of about 0.6 mM (Nadler et al., supra, Jiang et al., supra). The TRPM7 channel is also known as the CHAK, CHAK1, LTRPC7, FLJ20117 or TRP-PLIK channel. More recently, the TRPM7 channel has been shown to be involved in ischemic CNS injury and anoxic neuronal cell death (Aarts et al., The Neuroscientist 2005 11(2):116-123; Aarts et al., Cell 2003 115:863-877).

Very few effective therapies have been realized to treat ischemic injury, and in particular, stroke. The TRPM7 family represents a new and exciting target for therapies to treat these ischemic injuries. Although the human and mouse TRPM7 channels have been identified, rat is the animal of choice for animal models for developing drugs to treat ischemic disorders such as stroke. Accordingly, a needs exists for the identification of the rat TRPM7 gene and protein. The present invention meets this and other needs.

### SUMMARY OF THE INVENTION

The present invention thus provides, inter alia, nucleic acid and amino acid sequences of a rat TRPM7 gene isolated from rat and methods of screening for modulators of the rat TRPM7 gene.

In one aspect, the invention provides an isolated nucleic acid encoding at least one function domain of a rat transient receptor potential channel TRPM7. The nucleic acid comprises a nucleic acid sequence having at least 96% identity to SEQ ID NO:1, SEQ ID NO:5, SEQ ID NO:6 or SEQ ID NO:7. In some such aspects, the polynucleotide encodes a rat transient receptor potential channel TRPM7. In certain aspects, the nucleic acid comprises SEQ ID NO:1, SEQ ID NO:5, SEQ ID NO:6 or SEQ ID NO:7, their complements, or conservatively modified variants thereof.

In another aspect, the invention provides an isolated polypeptide comprising an amino acid sequence that has at least 99% sequence identity to SEQ ID NO:2.

In another aspect, the invention provides an ion transport domain comprising an amino acid sequence that has at least 99% sequence identity to SEQ ID NO:3.

In another aspect, the invention provides an alpha kinase domain comprising an amino acid sequence that has at least 99% sequence identity to SEQ ID NO:4.

In another aspect, the invention provides vectors, such as expression vectors, comprising a polynucleotide sequence of the invention. In other aspects, the invention provides host cells or progeny of the host cells comprising a vector of the invention. In certain aspects, the host cell is a eukaryote. In other aspects, the expression vector comprises a rat TRPM7 polynucleotide in which the nucleotide sequence of the polynucleotide is operatively linked with a regulatory sequence that controls expression of the polynucleotide in a host cell. In certain aspects, the invention provides a host cell comprising a rat TRPM7 polynucleotide, wherein the nucleotide sequence of the polynucleotide is operatively linked with a regulatory sequence that controls expression of the polynucleotide in a host cell, or progeny of the cell. The nucleotide sequence of the polynucleotide can be operatively linked to the regulatory sequence in a sense or antisense orientation.

In another aspect, the invention provides a method of producing a polypeptide comprising (i) culturing the host cell as described herein under conditions such that the polypeptide is expressed; and (ii) recovering the polypeptide from the cultured host cell of its cultured medium.

In another aspect, the invention provides a method for identifying a compound or agent that binds to a rat TRPM7 polypeptide comprising: (i) contacting a rat TRPM7 polypeptide with the compound or agent under conditions that allow binding of the compound to the rat TRPM7 polypeptide to form a complex and (ii) detecting the presence of the complex.

In another aspect, the invention provides a method of screening bioactive agents comprising: a) providing a cell that expresses a rat TRPM7 gene; b) adding a bioactive agent candidate to the cell; and c) determining the effect of the bioactive agent candidate on the expression of the rat TRPM7 gene. In some methods, the determining comprises comparing the level of expression in the absence of the bioactive agent candidate to the level of expression in the presence of the bioactive agent candidate.

In another aspect, the invention provides a method of screening for a bioactive agent capable of binding to a rat TRPM7 protein, the method comprising: a) combining the rat TRPM7 protein and a candidate bioactive agent; and b) determining the binding of the bioactive agent to the rat TRPM7 protein.

In another aspect, the invention provides a method for screening for a bioactive agent that increases or decreases ion flux through a rat TRPM7 channel, the method comprising: a) contacting a candidate bioactive agent with a TRPM7 protein wherein the TRPM7 protein forms a TRPM7 channel; and b) determining the functional effect of the bioactive agent on the TRPM7 channel. In one aspect, the determining step comprising comparing ion flux in the absence of the bioactive agent to ion flux in the presence of the bioactive agent.

In another aspect, the invention provides a method for screening for a bioactive agent that modulates the monovalent or divalent cationic permeability of a rat TRPM7 channel comprising a TRPM7 protein. The method comprises the steps of a) contacting the rat TRPM7 channel with a candidate bioactive agent; b) activating the channel; and c) detecting whether the bioactive agent modulates the monovalent or divalent cationic permeability of the channel. In one aspect, the monovalent or divalent cationic permeability of the channel is increased by contacting the channel with the bioactive agent. In another aspect, the monovalent or divalent cationic permeability of the channel is decreased by contacting the channel with the bioactive agent.

In another aspect, the invention provides a method for screening for a bioactive agent that modulates the monovalent or divalent cationic permeability of a channel comprising a TRPM7 protein comprising a) providing a recombinant cell comprising a recombinant nucleic acid encoding a TRPM7 protein and an inducible promoter operably linked thereto; b) inducing the recombinant cell to express the TRPM7 protein and form a channel comprising the TRPM7 protein; c) contacting the recombinant cell with a candidate bioactive agent; d) activating the channel; and e) detecting modulation of monovalent cation or divalent permeability of the channel. In one aspect, the monovalent or divalent cationic permeability of the channel is increased by contacting the cell with the bioactive agent. In another aspect, the monovalent or divalent cationic permeability of the channel is decreased by contacting the cell with the bioactive agent. In another aspect, contacting the channel with the bioactive agent alters the membrane potential of a cell comprising the TRPM7 protein.

In another aspect, the invention provides a method for screening for a bioactive agent that modulates the monovalent or divalent cationic permeability of a channel comprising a TRPM7 protein comprising a) providing a recombinant cell comprising a recombinant nucleic acid encoding a TRPM7 protein and an inducible promoter operably linked thereto; b) inducing the recombinant cell to express the TRPM7 protein and form a channel comprising the TRPM7 protein; c) contacting the recombinant cell with a candidate bioactive agent; d) activating the channel; and e) detecting the monovalent or divalent cation permeability of the channel. In one aspect, the detecting step comprises comparing the intracellular monovalent or divalent cation levels to intracellular monovalent or divalent cation levels in a cell that does not express the TRPM7 protein but that is in contact with the bioactive agent.

In another aspect, the invention provides an expression cassette comprising a polynucleotide encoding a rat TRPM7 polypeptide, wherein said polynucleotide is under the control of a promoter operable in eukaryotic cells. In some expression cassettes, the promoter is heterologous to the coding sequence. In some such expression cassettes, the promoter is a tissue specific promoter. In other such expression cassettes, the promoter is an inducible promoter. In some such expression cassette is contained in a viral vector. In some such expression cassettes, the viral vector is selected from the group consisting of a retroviral vector, an adenoviral vector, and adeno-associated viral vector, a vaccinia viral vector, and a herpesviral vector. Some such expression cassette further comprises a polyadenylation signal.

In another aspect, the invention provides a cell comprising an expression cassette comprising a polynucleotide encoding a rat TRPM7 polypeptide, wherein said polynucleotide is under the control of a promoter operable in eukaryotic cells, said promoter being heterologous to said polynucleotide.

In another aspect, the invention provides a method of increasing expression of a rat TRPM7 gene comprising the steps of (i) providing a biological system in which expression of a rat TRPM7 gene is to be increased; and (ii) contacting the system with an expression cassette comprising a polynucleotide encoding a rat TRPM7 polypeptide, wherein said polynucleotide is under the control of a promoter operable in eukaryotic cells; and (iii) inhibiting expression of the gene encoding the protein.

In another aspect, the invention provides a method of screening for a modulator of ischemic injury, said method comprising: contacting a recombinant cell or cell line that expresses the rat TRPM7 gene with a test compound; and detecting an increase or a decrease in the amount of cell death.

In another aspect, the invention provides a method of screening for a modulator of rat TRPM7 activity, the method comprising: contacting a cell or cell line as described herein with a test compound; and detecting an increase or a decrease in the amount of rat TRPM7 production, or rat TRPM7 activity, thereby identifying the test compound as a modulator of rat TRPM7 activity.

### BRIEF DESCRIPTION OF THE FIGURES

FIG. 1 display the gene structure, including the exon coordinates, of Rat TRPM7.

FIG. 2A-H shows an alignment of rat TRPM7 cDNA (SEQ ID NO:1) with the mouse TRPM7 cDNA (NM_{—}021450.1) (SEQ ID NO:14).

FIG. 3A-H shows an alignment of rat TRPM7 cDNA (SEQ ID NO:1) with the human TRPM7 cDNA (NM_{—}017672.2) (SEQ ID NO:15).

FIG. 4A-C shows an alignment of rat TRPM7 (SEQ ID NO:2) with mouse TRPM7 (SEQ ID NO:16). The consensus sequence (SEQ ID NO:17) shows conserved amino acids as “+” and blank spaces for non-conserved amino acids.

FIG. 5A-C shows an alignment of rat TRPM7 (SEQ ID NO:2) with human TRPM7 (SEQ ID NO:18). The consensus sequence (SEQ ID NO:19) shows conserved amino acids as “+” and blank spaces for non-conserved amino acids.

FIG. 6A-B shows full cDNA of Rat TRPM7 RNA (SEQ ID NO:5) with 5′ untranslated region shown in bold (also SEQ ID NO:8) and 3′ untranslated region shown in bold (also SEQ ID NO:9).

FIG. 7 shows the Rat TRPM7 protein (SEQ ID NO:2) in which the ion transport domain is underlined (also SEQ ID NO:3), and the alpha kinase domain is shown in grey shading (also SEQ ID NO:4). The nucleic acids encoding the ion transport domain and the alpha kinase domain are shown in SEQ ID NO:6 and SEQ ID NO:7, respectively.

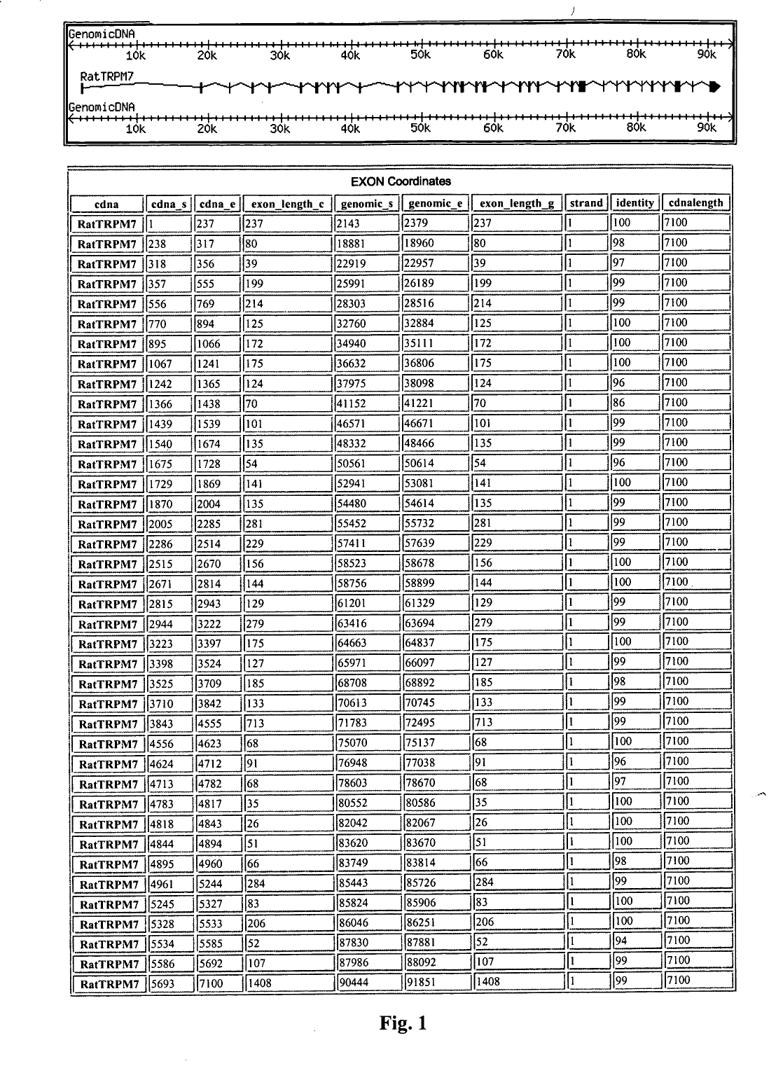

### DETAILED DESCRIPTION OF THE INVENTION

1. Introduction

The present invention provides, inter alia, a nucleic acid encoding TRPM7 identified and cloned from rat tissue. The rat TRPM7 polypeptide is a member of the TRP superfamily of cation channels.

The present invention also provides, inter alia, methods of screening for modulators (e.g., activators, inhibitors, stimulators, enhancers, agonists, and antagonists) of rat TRPM7 proteins. Such modulators can be used in the therapeutic treatment of ischemic injuries, including neurological diseases and conditions, such as stroke, Alzheimer's disease, Parkinson's disease, Huntington's disease, dementia, epilepsy, spinocerebellar ataxia, spinal and bulbar muscular dystrophy, dentatorubropallidoluysian atrophy, brain injury, spinal cord injury, and other traumatic nervous system injuries.

Functionally, the TRPM7 channel is, among other things, a Mg-ATP regulated divalent cation channel required for cell viability. It plays a significant role in neuronal cell death triggered by ischemic injuries.

Structurally, TRPM7, along with two other members of the TRP superfamily, can be distinguished from other known ion channels by their unusual architecture. They consist of enzyme domains linked to the C termini of ion channel domains

The isolation and characterization of TRPM7 from rat provides a means for assaying for modulators for TRPM7 activity that can then be tested in rat models of diseases, including rat models of diseases modulated by TRPM7 activity, including stroke.

It is to be understood that this invention is not limited to particular methods, reagents, compounds compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

“About” as used herein when referring to a measurable value such as an amount, a temporal duration, and the like, is meant to encompass variations of ±20% or ±110%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Rat TRPM7 protein” refers to an amino acid sequence that has at least 80%, at least 90%, at least 95%, preferably at least 99% amino acid sequence identity, including at least 99.1%, 99.2%, 99.3%, 99.4%, 99.5%, 99.6%, 99.7%, 99.8%, or at least 99.9%, identity to an amino acid sequence encoded by a rat TRPM7 nucleic acid, e.g., a rat TRPM7 protein as shown in SEQ ID NO:2.

“Nucleic acid encoding rat TRPM7 protein” or “TRPM7 gene” or “TRPM7 nucleic acid” refers to a nucleic acid sequence that has at least 96% nucleic acid sequence identity, or at least 90%, 95%, 96.5%, 97%, 97.5%, 98%, 98.5%, 99%, 99.1%, 99.2%, 99.3%, 99.4%, 99.5%, 99.6%, 99.7%, 99.8%, or at least 99.9%, to a rat TRPM7 nucleic acid as shown in SEQ ID NO:1, SEQ ID NO:5, their complements, or conservatively modified variants thereof.

Nucleic acid encoding at least one functional domain of a rat transient receptor potential channel TRPM7 refers to a nucleic acid sequence that has at least 96% nucleic acid sequence identity, or at least 90%, 95%, 96.5%, 97%, 97.5%, 98%, 98.5%, 99%, 99.1%, 99.2%, 99.3%, 99.4%, 99.5%, 99.6%, 99.7%, 99.8%, or at least 99.9%, to a rat TRPM7 nucleic acid as shown in SEQ ID NO:1, SEQ ID NO:5, SEQ ID NO:6, or SEQ ID NO:7, their complements, or conservatively modified variants thereof.

A rat transient receptor potential channel ion transport domain refers to an ion transport domain having amino acid sequence that has at least 80%, at least 90%, at least 95%, preferably at least 99% amino acid sequence identity, including at least 99.1%, 99.2%, 99.3%, 99.4%, 99.5%, 99.6%, 99.7%, 99.8%, or at least 99.9%, identity to SEQ ID NO:3. Activity of the ion transport domain can be determined using the methods described herein including patch clamp techniques.

A rat transient receptor potential channel alpha kinase domain refers to an alpha kinase domain having amino acid sequence that has at least 80%, at least 90%, at least 95%, preferably at least 99% amino acid sequence identity, including at least 99.1%, 99.2%, 99.3%, 99.4%, 99.5%, 99.6%, 99.7%, 99.8%, or at least 99.9%, identity to SEQ ID NO:4. Activity of the alpha kinase domain can be determined by measuring phosphorylation using well known techniques, including for example fluorescence resonance energy transfer based kinase assays, such as the LANCE™ assay available from Perkin Elmer (Schmitz et al., Journal of Biological Chemistry, Manuscript M509175200, Sep. 2, 2005).

The nucleic acids of the present invention preferably encode at least one functional domain of a rat transient receptor potential channel TRPM7, rat TRPM7, or functional equivalents thereof. In preferred embodiments, they do not encode human or mouse TRPM7 proteins including those represented by Genbank accession numbers NP_{—}060142 and NP_{—}067425. In preferred embodiments, the nucleic acids of the present invention do not include mouse or human TRPM7 nucleic acid sequences, including those represented by Genbank accession numbers NM_{—}017672.2 and NM_{—}021450.1.

A rat TRPM7 polynucleotide or polypeptide sequence can be naturally occurring or non-naturally occurring. It can be isolated from rat or synthetically constructed.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “functional effects” in the context of assays for testing compounds that affect a TRPM7 channel includes the determination of any parameter that is indirectly or directly under the influence of the channel. It includes changes in ion flux and membrane potential, changes in ligand binding, and other physiological effects such as increases or decreases in cell death following administration of a test compound.

By “determining the functional effect” refers to determining the functional effect of a compound on the TRPM7 channel. Such functional effects can be measured by any means known to those skilled in the art, e.g., patch clamping, voltage-sensitive dyes, whole cell currents, radioisotope efflux, inductive markers, and the like.

“Inhibitors,” “activators,” and “modulators” of rat TRPM7 genes and their gene products in cells refer to inhibitory or activating molecules identified using assays for TRPM7 channel function. Inhibitors are compounds that decrease, block, prevent, delay activation, inactivate, desensitize, or down regulate the channel. Activators are compounds that increase, open, activate, facilitate, enhance activation, sensitize or up regulate channel activity. Such assays for inhibitors and activators include e.g., expressing TRPM7 in cells or cell membranes and then measuring flux of ions through the channel and determining changes in polarization (i.e., electrical potential). Such assays also include measuring cell death after contacting a cell expressing TRPM7 with a putative modulator of TRPM7 activity. To examine the extent of inhibition, samples or assays comprising an TRPM7 channel are treated with a potential activator or inhibitor and are compared to control samples without the inhibitor. Control samples (untreated with inhibitors) are assigned a relative TRPM7 activity value of 100%. Inhibition of channels is achieved when the TRPM7 activity value relative to the control is about 90% or less, optionally about 80% or less, 70% or less, 60% or less, 50% or less, 40% or less, 30% or less; or 25-0%. Activation of channels comprising TRPM7 is achieved when the TRPM7 activity value relative to the control is about 110%, optionally 120%, 130%, 140%, 150% or more, 200-500% or more, 1000-3000% or more.

“Recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

A “constitutive” promoter is a promoter that is active under most environmental and developmental conditions. An “inducible” promoter is a promoter that is active under environmental or developmental regulation.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, streptavidin, digoxigenin, or haptens and proteins for which antisera or monoclonal antibodies are available (e.g., the polypeptide sequence as shown in the Figures can be made detectable, e.g., by incorporating a radiolabel into the peptide, and used to detect antibodies specifically reactive with the peptide).

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Identical” or percent “identity,” in the context of two or more nucleic acids or polypeptide sequences, refer to two or more sequences or subsequences that are the same or have a specified percentage of amino acid residues or nucleotides that are the same (i.e., 70% identity, preferably 75%, 80%, 85%, 90%, or 95% identity or higher over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length. In most preferred embodiments, the sequences are substantially identical over the entire length of, e.g., the coding region.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of (Smith et al., Adv. Appl. Math. 1981, 2:482), by the homology alignment algorithm of (Needleman et al., J. Mol. Biol. 1970, 48:443), by the search for similarity method of (Pearson et al., Proc. Nat'l. Acad. Sci. 1988, 85:2444), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology, 2005 supplement).

A preferred example of algorithm that is suitable for determining percent sequence identity and sequence similarity are the BLAST and BLAST 2.0 algorithms, which are described in (Altschul et al., Nuc. Acids Res. 1977, 25:3389-3402, and Altschul et al., J. Mol. Biol. 1990, 215: 403-410), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N-4 and a comparison of both strands. For amino acid sequences, the BLASTP program uses as defaults a wordlength of 3, and expectation (E) of 10, and the BLOSUM62 scoring matrix (see Henikoff et al., Proc. Natl. Acad. Sci. U.S.A. 1989, 89:10915) alignments (B) of 50, expectation (E) of 10, M=5, N=−4, and a comparison of both strands.

The BLAST algorithm also performs a statistical analysis of the similarity between two sequences (see, e.g., Karlin et al., Proc. Nat'l. Acad. Sci. 1993, 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of (Feng et al., J. Mol. Evol. 1987, 35:351-360). The method used is similar to the method described by (Higgins et al., CABIOS 1989, 5:151-153). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 1984, 12:387-395).

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The nucleic acids of the invention be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art (See, e.g., Sambrook, Tijssen and Ausubel discussed herein). In one example, a naturally occurring polynucleotide or polypeptide present in a living animal is not isolated, but the same polynucleotide or polypeptide, separated from some or all of the coexisting materials in the natural system, is isolated. Such polynucleotides can be part of a vector or part of a composition, and still be isolated in that such vector of composition is not part of its natural environment. As used herein, an isolated material or composition can also be a “purified” composition, i.e., it does not require absolute purity, rather, it is intended as a relative definition.

The nucleic acid sequences of the invention and other nucleic acids used to practice this invention, whether RNA, cDNA, genomic DNA, or hybrids thereof, can be isolated from a variety of sources, genetically engineered, amplified, and/or expressed recombinantly. Any recombinant expression system can be used, including, in addition to bacterial, e.g., yeast, insect or mammalian systems. Alternatively, these nucleic acids can be chemically synthesized in vitro. Techniques for the manipulation of nucleic acids, such as, e.g., subcloning into expression vectors, labeling probes, sequencing, and hybridization are well described in the scientific and patent literature, see, e.g., Sambrook, Tijssen and Ausubel. Nucleic acids can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, such as fluid or gel precipitin reactions, immunodiffusion (single or double), immunoelectrophoresis, adioimmunoassay (RIAs), enzyme-linked immunosorbent assays (ELISAs), immuno-fluorescent assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

“Recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins 1984).

Macromolecular structures such as polypeptide structures can be described in terms of various levels of organization. For a general discussion of this organization, (see, e.g., Alberts et al., Molecular Biology of the Cell 3: 1994) and (Cantor et al., Biophysical Chemistry Part I. The Conformation of Biological Macromolecules, 1980). “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity, e.g., a kinase domain. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript can be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are contemplated here.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in (Tijssen, Techniques in Biochemistry and Molecular Biology—Hybridization with Nucleic Probes, 1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

An intact “antibody” comprises at least two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. The term antibody includes antigen-binding portions of an intact antibody that retain capacity to bind TRPM7. Examples of binding include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 1989, 341:544-546), which consists of a VH domain; and (vi) an isolated complementarity determining region (CDR).

“Single chain antibodies” or “single chain Fv (scFv)” refers to an antibody fusion molecule of the two domains of the Fv fragment, VL and VH. Although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); (See, e.g., Bird et al., Science 1988, 242: 423-426; and Huston et al., Proc. Natl. Acad. Sci. 1988, 85: 5879-5883). Such single chain antibodies are included by reference to the term “antibody” fragments can be prepared by recombinant techniques or enzymatic or chemical cleavage of intact antibodies.

“Human sequence antibody” includes antibodies having variable and constant regions (if present) derived from human germline immunoglobulin sequences. The human sequence antibodies of the invention can include amino acid residues not encoded by human germline immunoglobulin sequences (e.g., mutations introduced by random or site-specific mutagenesis in vitro or by somatic mutation in vivo). Such antibodies can be generated in non-human transgenic animals, e.g., as described in PCT Publication Nos. WO 01/14424 and WO 00/37504. However, the term “human sequence antibody”, as used herein, is not intended to include antibodies in which CDR sequences derived from the germline of another mammalian species, such as a mouse, have been grafted onto human framework sequences (e.g., humanized antibodies).

Also, recombinant immunoglobulins may be produced. (See, Cabilly, U.S. Pat. No. 4,816,567); and (Queen et al., Proc. Nat'l Acad. Sci. 1989, 86: 10029-10033).

“Monoclonal antibody” refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions (if present) derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

“Polyclonal antibody” refers to a preparation of more than 1 (two or more) different antibodies to a TRPM7 protein. Such a preparation includes antibodies binding to a range of different epitopes. Antibodies to TRPM7 can bind to an epitope on rat TRPM7 so as to inhibit TRPM7. These and other antibodies suitable for use in the present invention can be prepared according to methods that are well known in the art and/or are described in the references cited here.

For preparation of monoclonal or polyclonal antibodies to polypeptides of the present invention, any technique known in the art can be used (see, e.g., Kohler et al., Nature 1975, 256: 495-497; Kozbor et al., Immunology Today 1983, 4: 72; Cole et al., Monoclonal Antibodies and Cancer Therapy, 1985, 77-9). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 1990, 348: 552-554; Marks et al., Biotechnology 1992, 10: 779-783).

An “anti-rat TRPM7” antibody is an antibody or antibody fragment that specifically binds a polypeptide encoded by the rat TRPM7 gene, cDNA, or a subsequence thereof.

An “immunoassay” is an assay that uses an antibody to specifically bind an antigen. The immunoassay is characterized by the use of specific binding properties of a particular antibody to isolate, target, and/or quantify the antigen.

“A monovalent cation indicator” refers to a molecule that is readily permeable to a cell membrane or otherwise amenable to transport into a cell e.g., via liposomes, etc., and upon entering a cell, exhibits a fluorescence signal, or other detectable signal, that is either enhanced or quenched upon contact with a monovalent cation. Examples of monovalent cation indicators useful in the invention are set out in Haugland, R. P. Handbook of Fluorescent Probes and Research Chemicals., 9th ed. Molecular Probes, Inc Eugene, Oreg., (2001).

“A divalent cation indicator” refers to a molecule that is readily permeable to a cell membrane or otherwise amenable to transport into a cell e.g., via liposomes, etc., and upon entering a cell, exhibits a fluorescence signal, or other detectable signal, that is either enhanced or quenched upon contact with a divalent cation.

“Specifically bind(s)” or “bind(s) specifically” when referring to a peptide refers to a peptide molecule which has intermediate or high binding affinity, exclusively or predominately, to a target molecule. The phrases “specifically binds to” refers to a binding reaction which is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions can require a binding moiety that is selected for its specificity for a particular target antigen. A variety of assay formats can be used to select ligands that are specifically reactive with a particular protein. For example, solid-phase ELISA immunoassays, immunoprecipitation, Biacore and Western blot are used to identify peptides that specifically react with the antigen. Typically a specific or selective reaction will be at least twice background signal or noise and more typically more than 10 times background.

“Naturally-occurring” as applied to an object refers to the fact that an object can be found in nature. For example, a polypeptide or polynucleotide sequence that is present in an organism (including viruses) that can be isolated from a source in nature and which has not been intentionally modified in the laboratory is naturally-occurring.

This invention relies on routine techniques in the field of recombinant genetics. Basic texts disclosing the general methods of use in this invention include (Sambrook et al., Molecular Cloning, A Laboratory Manual, 2: 1989; Kriegler, Gene Transfer and Expression: A Laboratory Manual, 1990; and Ausubel et al., Current Protocols in Molecular Biology, 2005).

Rat TRPM7 genes, nucleic acids and polymorphic variants that are substantially identical to sequences provided herein, can be isolated using rat TRPM7 nucleic acid probes and oligonucleotides under stringent hybridization conditions, by screening libraries. Alternatively, expression libraries can be used to clone rat TRPM7 protein, including polymorphic variants, by detecting expressed homologs immunologically with antisera or purified antibodies made against rat TRPM7 genes and their gene products or portions thereof.

2. Isolation of the Gene Encoding Rat TRPM7

A. General Recombinant DNA Methods

The nucleic acids used to practice this invention can be isolated from rat tissue, genetically engineered, amplified, and/or expressed/generated recombinantly. Recombinant polypeptides generated from these nucleic acids can be individually isolated or cloned and tested for a desired activity. Any recombinant expression system can be used.

Alternatively, these nucleic acids can be synthesized in vitro by well-known chemical synthesis techniques, as described in, (e.g., Adams, J. Am. Chem. Soc. 1983, 105:661; Belousov, Nucleic Acids Res. 1997, 25:3440-3444; Frenkel, Free Radic. Biol. Med. 1995, 19: 373-380; Blommers, Biochemistry 1994, 33:7886-7896; Narang, Meth. Enzymol. 1979 68:90; Brown Meth. Enzymol. 1979, 68:109; Beaucage, Tetra. Lett. 1981, 22:1859; U.S. Pat. No. 4,458,066).

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, (see, e.g., Sambrook, 1989 and Ausubel, 2005, supra).

Rat TRPM7 polypeptides and nucleic acids are used in the assays described herein. For example, recombinant rat TRPM7 can be used to make cells that constitutively express rat TRPM7. Such polypeptides and nucleic acids can be made using routine techniques in the field of recombinant genetics.

Oligonucleotides that are not commercially available can be chemically synthesized according to the solid phase phosphoramidite triester method first described by (Beaucage et al., Tetrahedron Letts. 1981, 22: 1859-1862, 1981), using an automated synthesizer, as described in (Van Devanter et al., Nucleic Acids Res. 1984, 12: 6159-6168). Purification of oligonucleotides is typically by either native acrylamide gel electrophoresis or by anion-exchange HPLC as described in (Pearson et al., J. Chrom. 1983, 255: 137-149). The sequence of the cloned genes and synthetic oligonucleotides can be verified after cloning using, e.g., the chain termination method for sequencing double-stranded templates of (Wallace et al., Gene 1981, 16:21-26). Again, as noted above, companies such as Operon Technologies, Inc. provide an inexpensive commercial source for essentially any oligonucleotide.

The sequence of the cloned genes and synthetic oligonucleotides can be verified after cloning using, e.g., the chain termination method for sequencing double-stranded templates of Wallace et al., Gene 1981, 16:21-26.

B. Cloning Methods for the Isolation of Nucleotide Sequences Encoding Rat TRPM7

In general, the nucleic acid sequences encoding genes of interest, such as rat TRPM7, are cloned from cDNA and genomic DNA libraries by hybridization with a probe, or isolated using amplification techniques with oligonucleotide primers. Rat TRPM7 sequences are typically isolated from rat nucleic acid (genomic or cDNA) libraries by hybridizing with a nucleic acid probe, the sequence of which can be derived from the sequences provided herein.

Amplification techniques using primers can also be used to amplify and isolate, e.g., a nucleic acid encoding rat TRPM7, from DNA or RNA (see, e.g., Dieffenfach et al., PCR Primer: A Laboratory Manual, 1995). These primers can be used, e.g., to amplify either the full length sequence or a probe of one to several hundred nucleotides, which is then used to screen a mammalian library for the full-length nucleic acid of choice. For example, degenerate primer sets can be used to isolate rat TRPM7 nucleic acids. Nucleic acids can also be isolated from expression libraries using antibodies as probes. Such polyclonal or monoclonal antibodies can be raised, e.g., using the sequence of SEQ ID NO:2.

To make a cDNA library, one should choose a source that is rich in TRPM7 mRNA. The mRNA is then made into cDNA using reverse transcriptase, ligated into a recombinant vector, and transfected into a recombinant host for propagation, screening and cloning. Methods for making and screening cDNA libraries are well known (see, e.g., Gubler et al., Gene 1983, 25: 263-269; Sambrook et al., supra; Ausubel et al., supra).

For a genomic library, the DNA is extracted from the tissue and either mechanically sheared or enzymatically digested to yield fragments of about 12-20 kb. The fragments are then separated by gradient centrifugation from undesired sizes and are constructed in non-lambda expression vectors. These vectors are packaged in vitro. Recombinant phage are analyzed by plaque hybridization as described in (Benton et al., Science 1977, 196:180-182). Colony hybridization is carried out as generally described in (Grunstein et al., Proc. Natl. Acad. Sci. U.S.A. 1975 72:3961-3965).

An alternative method of isolating a nucleic acid and its homologs combines the use of synthetic oligonucleotide primers and amplification of an RNA or DNA template (see U.S. Pat. Nos. 4,683,195 and 4,683,202; Innis et al., PCR Protocols: A Guide to Methods and Applications, 1990). Methods such as polymerase chain reaction (PCR) and ligase chain reaction (LCR) can be used to amplify nucleic acid sequences of, e.g., rat TRPM7 directly from mRNA, from cDNA, from genomic libraries or cDNA libraries. Degenerate oligonucleotides can be designed to amplify rat TRPM7 homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of TRPM7 encoding mRNA in physiological samples, for nucleic acid sequencing, or for other purposes. Genes amplified by the PCR reaction can be purified from agarose gels and cloned into an appropriate vector.

As described above, gene expression of rat TRPM7 can also be analyzed by techniques known in the art, e.g., reverse transcription and PCR amplification of mRNA, isolation of total RNA or poly A⁺ RNA, northern blotting, dot blotting, in situ hybridization, RNase protection, probing high density oligonucleotides, and the like. All of these techniques are standard in the art.

Synthetic oligonucleotides can be used to construct recombinant genes for use as probes or for expression of protein. This method is performed using a series of overlapping oligonucleotides usually 40-120 bp in length, representing both the sense and non-sense strands of the gene. These DNA fragments are then annealed, ligated and cloned. Alternatively, amplification techniques can be used with precise primers to amplify a specific subsequence of the TRPM7 nucleic acid. The specific subsequence is then ligated into an expression vector.

The nucleic acid encoding the protein of choice is typically cloned into intermediate vectors before transformation into prokaryotic or eukaryotic cells for replication and/or expression. These intermediate vectors are typically prokaryote vectors, e.g., plasmids, or shuttle vectors. Optionally, cells can be transfected with recombinant TRPM7 operably linked to a constitutive promoter, to provide higher levels of TRPM7 expression in cultured cells.

C. Expression in Prokaryotes and Eukaryotes

To obtain high level expression of a cloned gene or nucleic acid, such as those cDNAs encoding rat TRPM7, one typically subclones rat TRPM7 into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al. and Ausubel et al. Kits for such expression systems are commercially available. Eukaryotic expression systems for mammalian cells, including rat, are well known in the art and are also commercially available.

The promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function. The promoter typically can also include elements that are responsive to transactivation, e.g., hypoxia responsive elements, Gal4 responsive elements, lac repressor responsive elements, and the like. The promoter can be constitutive or inducible, heterologous or homologous.

In addition to the promoter, the expression vector typically contains a transcription unit or expression cassette that contains all the additional elements required for the expression of the nucleic acid in host cells. A typical expression cassette thus contains a promoter operably linked, e.g., to the nucleic acid sequence encoding rat TRPM7, and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as using a baculovirus vector in insect cells, with a rat TRPM7 encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, including rat, yeast or insect cell lines that express large quantities of protein, which are then purified using standard techniques (see, e.g., Colley et al., J. Biol. Chem. 1989, 264:17619-17622; Deutscher, Guide to Protein Purification, in Methods in Enzymology, 1990, 182). Transformation of eukaryotic and prokaryotic cells are performed according to standard techniques (see, e.g., Morrison, J. Bact. 1977, 132: 349-351; Clark-Curtiss et al., Methods in Enzymology 101: 347-362, and Wu et al., 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

After the expression vector is introduced into the cells, the transfected cells are cultured under conditions favoring expression of the rat TRPM7 protein, which can be recovered from the culture using standard techniques identified below.

3. Peptides and Polypeptides

The invention provides, inter alia, methods for inhibiting the activity of rat TRPM7 polypeptides, e.g., a polypeptide of the invention. The invention also provides, inter alia, methods for screening for compositions that inhibit the activity of, or bind to (e.g., bind to the active site) rat TRPM7 polypeptides.

The peptides and polypeptides of the invention can be expressed recombinantly in vitro or in vivo to screen for modulators of a rat TRPM7 activity and for agents that protect cells from oxygen and/or glucose deprivation.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., (Caruthers, Nucleic Acids Res. Symp. Ser. 215-223, 1980; Horn, Nucleic Acids Res. Symp. Ser. 225-232, 1990; Banga, Therapeutic Peptides and Proteins, Formulation, Processing and Delivery Systems, 1995). For example, peptide synthesis can be performed using various solid-phase techniques (see e.g., Roberge, Science 269: 202, 1995; Merrifield, Methods Enzymol. 289: 3-13, 1997) and automated synthesis can be achieved, e.g., using the ABI 431A Peptide Synthesizer (Perkin Elmer) in accordance with the instructions provided by the manufacturer.

A polypeptide can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholin-yl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (e.g., containing the CN-moiety in place of COOH) can be substituted for Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of e.g., pipecolic acid, thiazolidine carboxylic acid, 3- or 4-hydroxy e.g., diethylprocarbonate or para-bromophenacyl bromide. Other mimetics include, e.g., those generated by hydroxylation of

A component of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R- or S-form

The skilled artisan will recognize that individual synthetic residues and polypeptides incorporating these mimetics can be synthesized using a variety of procedures and methodologies, which are well described in the scientific and patent literature, e.g., (Gilman et al., Organic Syntheses Collective). Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., (al-Obeidi, Mol. Biotechnol. 1998, 9: 205-223; Hruby, Curr. Opin. Chem. Biol. 1997, 1: 114-119; Ostergaard, Mol. Divers. 1997, 3: 17-27; Ostresh, Methods Enzymol. 1996, 267: 220-234). Modified peptides of the invention can be further produced by chemical modification methods, see, e.g., (Belousov Nucleic Acids Res. 1997, 25: 3440-3444; Frenkel, Free Radic. Biol. Med. 1995, 19: 373-380; Blommers, Biochemistry 1994, 33: 7886-7896).

Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams, Biochemistry 1995, 34: 1787-1797; Dobeli, Protein Expr. Purif. 1998, 12: 404-14). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, (see e.g., Kroll, DNA Cell. Biol. 1993, 12: 441-53).

In practicing the methods of the invention, a variety of apparatus and methodologies can be used to in conjunction with the polypeptides and nucleic acids of the invention, e.g., to screen polypeptides for rat TRPM7 activity, to screen compounds as potential modulators (e.g., inhibitors or activators) of a rat TRPM7 activity, for antibodies that bind to a polypeptide of the invention, for nucleic acids that hybridize to a nucleic acid of the invention, to screen for cells expressing a polypeptide of the invention and the like.

In one aspect, the peptides and polypeptides of the invention can be bound to a solid support. Solid supports can include, e.g., membranes (e.g., nitrocellulose or nylon), a microtiter dish (e.g., PVC, polypropylene, or polystyrene), a test tube (glass or plastic), a dip stick (e.g., glass, PVC, polypropylene, polystyrene, latex and the like), a microfuge tube, or a glass, silica, plastic, metallic or polymer bead or other substrate such as paper. One solid support uses a metal (e.g., cobalt or nickel)-comprising column which binds with specificity to a histidine tag engineered onto a peptide.

Adhesion of peptides to a solid support can be direct (i.e., the protein contacts the solid support) or indirect (a particular compound or compounds are bound to the support and the target protein binds to this compound rather than the solid support). Peptides can be immobilized either covalently (e.g., utilizing single reactive thiol groups of cysteine residues (see, e.g., Colliuod Bioconjugate Chem. 1993, 4: 528-536) or non-covalently but specifically (e.g., via immobilized antibodies (see, e.g., Schuhmann, Adv. Mater. 1991, 3:388-391; Lu, Anal. Chem. 1995, 67: 83-87); the biotin/strepavidin system (see, e.g., Iwane, Biophys. Biochem. Res. Comm. 1997, 230: 76-80); metal chelating, e.g., Langmuir-Blodgett films (see, e.g., Ng, Langmuir 1995, 11: 4048-55); metal-chelating self-assembled monolayers (see, e.g., Sigal, Anal. Chem. 1996, 68:490-497) for binding of polyhistidine fusions.

Indirect binding can be achieved using a variety of linkers which are commercially available. The reactive ends can be any of a variety of functionalities including, but not limited to: amino reacting ends such as N-hydroxysuccinimide (NHS) active esters, imidoesters, aldehydes, epoxides, sulfonyl halides, isocyanate, isothiocyanate, and nitroaryl halides; and thiol reacting ends such as pyridyl disulfides, maleimides, thiophthalimides, and active halogens. The heterobifunctional crosslinking reagents have two different reactive ends, e.g., an amino-reactive end and a thiol-reactive end, while homobifunctional reagents have two similar reactive ends, e.g., bismaleimidohexane (BMH) which permits the cross-linking of sulfhydryl-containing compounds. The spacer can be of varying length and be aliphatic or aromatic. Examples of commercially available homobifunctional cross-linking reagents include, but are not limited to, the imidoesters such as dimethyl adipimidate dihydrochloride (DMA); dimethyl pimelimidate dihydrochloride (DMP); and dimethyl suberimidate dihydrochloride (DMS). Heterobifunctional reagents include commercially available active halogen-NHS active esters coupling agents such as N-succinimidyl bromoacetate and N-succinimidyl (4-iodoacetyl)aminobenzoate (SLAB) and the sulfosuccinimidyl derivatives such as sulfosuccinimidyl(4-iodoacetyl)aminobenzoate (sulfo-SIAB) (Pierce). Another group of coupling agents is the heterobifunctional and thiol cleavable agents such as N-succinimidyl 3-(2-pyridyidithio)propiona-te (SPDP) (Pierce Chemicals, Rockford, Ill.).

Antibodies can be used for binding polypeptides and peptides of the invention to a solid support. This can be done directly by binding peptide-specific antibodies to the column or it can be done by creating fusion protein chimeras comprising motif-containing peptides linked to, e.g., a known epitope (e.g., a tag (e.g., FLAG, myc) or an appropriate immunoglobulin constant domain sequence (an “immunoadhesin,” see, e.g., Capon, Nature 1989, 377: 525-531).

4. Purification of Rat TRPM7 Polypeptides

If necessary, naturally occurring or recombinant proteins can be purified for use in functional assays. Recombinant rat TRPM7 can be purified from any suitable expression system, e.g., by expressing rat TRPM7 in E. coli and then purifying the recombinant protein via affinity purification, e.g., by using antibodies that recognize a specific epitope on the protein or on part of the fusion protein, or by using glutathione affinity gel, which binds to GST. In some embodiments, the recombinant protein is a fusion protein, e.g., with GST or Gal4 at the N-terminus.

The protein of choice may be purified to substantial purity by standard techniques, including selective precipitation with such substances as ammonium sulfate; column chromatography, immunopurification methods, and others (see, e.g., Scopes, Protein Purification: Principles and Practice, 1982; U.S. Pat. No. 4,673,641; Ausubel et al., supra; and Sambrook et al., supra).

A number of procedures can be employed when recombinant protein is being purified. For example, proteins having established molecular adhesion properties can be reversibly fused to rat TRPM7. With the appropriate ligand, rat TRPM7 can be selectively adsorbed to a purification column and then freed from the column in a relatively pure form. The fused protein is then removed by enzymatic activity. Finally, rat TRPM7 could be purified using immunoaffinity columns.

A. Purification of Rat TRPM7 from Recombinant Bacteria

Recombinant proteins can be expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein.

Proteins expressed in bacteria may form insoluble aggregates (“inclusion bodies”). Several protocols are suitable for purification of inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. The protein of choice is separated from other bacterial proteins by standard separation techniques, e.g., with Ni-NTA agarose resin.

Alternatively, it is possible to purify the recombinant rat TRPM7 protein from bacteria periplasm. After lysis of the bacteria, when the protein is exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques for Purifying Rat TRPM7

Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size Differential Filtration

The molecular weight of the protein, e.g., rat TRPM7, can be used to isolated the protein from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

Column Chromatography

The protein of choice can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

5. Immunological Detection of Rat TRPM7

In addition to the detection of rat TRPM7 genes and gene expression using nucleic acid hybridization technology, one can also use, for example, immunoassays to detect rat TRPM7. Immunoassays can be used to qualitatively or quantitatively analyze rat TRPM7, e.g., to detect rat TRPM7, to measure rat TRPM7 activity, or to identify modulators of rat TRPM7 activity. A general overview of the applicable technology can be found in Harlow et al., (Antibodies: A Laboratory Manual, 1988).

A. Antibodies to Rat TRPM7

Methods of producing polyclonal and monoclonal antibodies that react specifically with a particular protein are known to those of skill in the art (see, e.g., Coligan, Current Protocols in Immunology, 1991; Harlow et al., supra; Goding, Monoclonal Antibodies: Principles and Practice 2: 1986; and Kohler et al. Nature 1975, 256:495-497). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 1989, 246: 1275-1281; Ward et al., Nature 1989, 341: 544-546). In addition, as noted above, many companies, such as BMA Biomedicals, Ltd., HTI Bio-products, and the like, provide the commercial service of making an antibody to essentially any peptide.

Monoclonal antibodies may be obtained by various techniques familiar to those skilled in the art. Briefly, spleen cells from an animal immunized with a desired antigen are immortalized, commonly by fusion with a myeloma cell (see Kohler et al. Eur. J. Immunol. 1976 6: 511-519). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by (Huse et al., Science 1989, 246: 1275-1281).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-rat TRPM7 proteins or even other related proteins, e.g., from other organisms, using a competitive binding immunoassay. Specific polyclonal antisera and monoclonal antibodies will usually bind with KD of at least about 0.1 mM, more usually at least about 1 .mu.M, preferably at least about 0.1 .mu.M or better, and most preferably, 0.01 .mu.M or better.

Once rat TRPM7 specific antibodies are available, these proteins can be detected by a variety of immunoassay methods. For a review of immunological and immunoassay procedures, see Basic and Clinical Immunology (Stites et al., 7: 1991). Moreover, the immunoassays of the present invention can be performed in any of several configurations, which are reviewed extensively in Enzyme Immunoassay (Maggio, 1980; and Harlow et al., supra).

B. Immunological Binding Assays

Rat TRPM7 can be detected and/or quantified using any of a number of well recognized immunological binding assays (see, e.g., U.S. Pat. Nos. 4,366,241; 4,376,110; 4,517,288; and 4,837,168, see also Asai, Methods in Cell Biology: Antibodies in Cell Biology, 37: 1993; Stites et al., Basic and Clinical Immunology 7, 1991). Immunological binding assays (or immunoassays) typically use an antibody that specifically binds to a protein or antigen of choice (in this case rat TRPM7, or antigenic fragments thereof). The antibody may be produced by any of a number of means well known to those of skill in the art and as described herein.

Immunoassays also often use a labeling agent to specifically bind to and label the complex formed by the antibody and antigen. The labeling agent may itself be one of the moieties comprising the antibody/antigen complex. Thus, the labeling agent may be a labeled rat TRPM7 polypeptide or a labeled anti-rat TRPM7. Alternatively, the labeling agent may be a third moiety, such a secondary antibody, that specifically binds to the antibody/antigen complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol. 1973, 111: 1401-1406; Akerstrom et al., J. Immunol. 1985, 135: 2589-2542). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Non-Competitive Assay Formats

Immunoassays for detecting rat TRPM7 in samples may be either competitive or noncompetitive. Noncompetitive immunoassays are assays in which the amount of antigen is directly measured. In one preferred “sandwich” assay, for example, the anti-antigen antibodies can be bound directly to a solid substrate on which they are immobilized. These immobilized antibodies then capture antigen present in the test sample. Antigen thus immobilized is then bound by a labeling agent, such as a second antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

Competitive Assay Formats

In competitive assays, the amount of rat TRPM7 present in the sample is measured indirectly by measuring the amount of a known, added (exogenous) antigen displaced (competed away) from an anti-antigen antibody by the unknown antigen present in a sample. In one competitive assay, a known amount of antigen is added to a sample and the sample is then contacted with an antibody that specifically binds to the antigen. The amount of exogenous antigen bound to the antibody is inversely proportional to the concentration of antigen present in the sample. In a particularly preferred embodiment, the antibody is immobilized on a solid substrate. The amount of antigen bound to the antibody may be determined either by measuring the amount of antigen present in an antigen/antibody complex, or alternatively by measuring the amount of remaining uncomplexed protein. The amount of antigen may be detected by providing a labeled antigen molecule.

A hapten inhibition assay is another preferred competitive assay. In this assay the known antigen is immobilized on a solid substrate. A known amount of anti-antigen antibody is added to the sample, and the sample is then contacted with the immobilized antigen. The amount of anti-antigen antibody bound to the known immobilized antigen is inversely proportional to the amount of antigen present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Cross-Reactivity Determinations

Immunoassays in the competitive binding format can also be used for crossreactivity determinations. For example, rat TRPM7 proteins can be immobilized to a solid support. Proteins are added to the assay that compete for binding of the antisera to the immobilized antigen. The ability of the added protein to compete for binding of the antisera to the immobilized protein is compared to the ability of antigen to compete with itself. The percent crossreactivity for the above proteins is calculated, using standard calculations. Those antisera with less than 10% crossreactivity with the added proteins are selected and pooled. The cross-reacting antibodies are optionally removed from the pooled antisera by immunoabsorption with the added proteins.

The immunoabsorbed and pooled antisera are then used in a competitive binding immunoassay as described above to compare a second protein thought to be perhaps an allele, interspecies homologs, or polymorphic variant of rat TRPM7, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the first protein that is required to inhibit 50% of binding, then the second protein is said to specifically bind to the polyclonal antibodies generated to the immunogen of choice.

Other Assay Formats

Western blot (immunoblot) analysis can be used to detect and quantify the presence of rat TRPM7 in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind rat TRPM7. The anti-antigen antibodies specifically bind to the antigen on the solid support. These antibodies may be directly labeled or alternatively may be subsequently detected using labeled antibodies (e.g., labeled sheep anti-mouse antibodies) that specifically bind to the anti-antigen antibodies.

Other assay formats include, for example, liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 1986, 5: 34-41).

Reduction of Non-Specific Binding

One of skill in the art will appreciate that it is often desirable to minimize non-specific binding in immunoassays. Particularly where the assay involves an antigen or antibody immobilized on a solid substrate, it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Labels

The particular label or detectable group used in this assay or any other assay of the present invention is not a critical aspect of the invention. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS®), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic beads (e.g., polystyrene, polypropylene, latex, and the like).

The label can be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize a specific protein, or secondary antibodies that recognize antibodies to the specific protein.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, and the like Chemiluminescent compounds include luciferin, and 2,3-dihydrophthalazined-iones, e.g., luminol. For a review of various labeling or signal producing systems that may be used, see (U.S. Pat. No. 4,391,904).

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

6. Assays for Modulators of Rat TRPM7 Production or Activity

TRPM7 has been identified as a MgATP regulated and calcium-permeant ion channel required for cell viability. As an ion channel, TRPM7 conducts calcium and monovalent cations to depolarize cells and increase intracellular calcium. TRPM7 currents are activated at low MgATP levels and are blocked by a number of divalent and polyvalent cations, including magnesium, zinc, spermine, 2-aminophenoxyborate, Mn(III) tetrakis (4-benzoic acid) porphyrin chloride, and lanthanum (Harteneck, Arch Pharamcol 2005 371″307-314). Both Mg²⁺ and Zn² permeate TRPM7 channels and block the movovalent cation flow through them (Kozak et al., Biophys. 2003 84:2293-2305). The TRPM7 channel produces pronounced outward currents at nonphysiological voltages ranging from +50 to +100 mV and small inward currents at negative potentials between −100 to −40 mV when expressed heterologously in mammalian cells (Jiang et al, J. Gen. Physiol. 2005 126(2), 137-150) TRPM7 has also been shown to be modulated by Src-family kinases (Jiang et al., J. Biol. Chem. 2003 278:42867-42876), phosphatidylinositol 4,5-biphosphate (PIP₂) (Runnels et al., Nat Cell Biol 2002 4:329-336), and its own α-kinase domain (Takezawa et al., PNAS USA 2004 101:6009-6014). Heterologously expressed TRPM7 channels, e.g., TPRM7 channels expressed in HEK-293 cells, exhibit currents with a high Ca²⁺ permeability, an outwardly rectifying I-V curve, enhancement by low Ca²⁺ concentration and a block of current by the polyvalent cation gadolinium. Overexpression of TRPM7 channels has been shown to be lethal to HEK-293 cells. The lethality can be prevented by increasing extracellular Mg²⁺ to restore Mg²⁺ homeostasis (Aarts et al., Cell 2003 115:863-877).

TRPM7 channels have been demonstrated to play a key role in neuronal cell death, and, in particular in anoxic cell death. TRPM7 is a mediator of ischemic induced toxicity and, in particular, ischemia induced injury activated by oxidative stress. Suppression of TRPM7 has been shown to increase survival of neuronal cells following ischemia induced toxicity. (Aarts et al., The Neuroscientist 2005 11(2):116-123; Aarts et al., Cell 2003 115:863-877)

The present invention provides, inter alia, cell based systems that can be used to identify modulators, for example, inhibitors or activators of TRPM7 production or activity. The amount or activity of a TRPM7 channel can be assessed using a variety of assays, including measuring current, measuring membrane potential, measuring ion flux, measuring ligand binding, measuring second messengers and transcription levels or physiological effects such as cell survival.

Modulators of the TRPM7 channels can be tested using biologically active TRPM7, either recombinant or naturally occurring. Rat TRPM7 can be isolated, co-expressed or expressed in a cell, or expressed in a membrane derived from a cell. Samples or assays that are treated with a potential TRPM7 channel inhibitor or activator can be compared to control samples without the test compound, to examine the extent of modulation. Control samples (untreated with activators or inhibitors) are assigned a relative TRPM7 activity value of 100%. Inhibition of channels comprising TRPM7 is achieved when the ion channel activity value relative to the control is, for example, about 90%, preferably about 50%, more preferably about 25%. Activation of channels comprising TRPM7 is achieved when the ion channel activity value relative to the control is 110%, more preferably 150%, more preferable 200% higher.

Changes in ion flux can be assessed by determining changes in polarization (i.e., electrical potential) of the cell membrane expressing the TRPM7 channel. A preferred means to determine changes in cellular polarization is by measuring changes in current (thereby measuring changes in polarization) with voltage-clamp and patch-clamp techniques, e.g., the “cell-attached” mode, the “inside-out” mode, and the “whole cell” mode (see, e.g., Runnels et al. Science 2001 291:1043-1047, Jiang et al, J. Gen. Physiol. 2005 126(2), 137-150). Whole cell currents are conveniently determined using the standard methodology (see, e.g., Hamil et al., PFlugers. Archiv. 1981, 391:85). Other known assays include: radiolabeled rubidium flux assays and fluorescence assays using ion-sensitive dyes, voltage-sensitive dyes (see, e.g., Vestergarrd-Bogind et al., J. Membrane Biol. 1988, 88:67-75; Daniel et al., J. Pharmacol. Meth. 1991, 25:185-193; Holevinsky et al., J Membrane Biology 1994, 137:59-70). Generally, the compounds to be tested are present in the range from about 1 pM to about 100 mM.

The present invention provides, inter alia, methods of identifying molecules that bind TRPM7, methods of identifying molecules that modulate TRPM7 ion channel activity, and/or methods of identifying molecules that alter expression of TRPM7 within a cell. These molecules are candidate bioactive agents that can be useful for treating conditions or diseases regulated by TRPM7 ion channel activity, e.g., stroke, Alzheimer's disease, Parkinson's disease, Huntington's disease, dementia, epilepsy, spinocerebellar ataxia, spinal and bulbar muscular dystrophy, dentatorubropallidoluysian atrophy, brain injury, spinal cord injury, and other traumatic nervous system injuries. In a preferred embodiment, these methods can be used to identify drug candidates that inhibit rat TRPM7 channel activity.

The present invention provides methods of screening for a candidate bioactive agent capable of binding to TRPM7. In some embodiments, the candidate bioactive agent will bind to a particular domain of the TRPM7 protein, such as, the C-terminal kinase domain.

In one embodiment for binding assays, either TRPM7 or a candidate bioactive agent is labeled. The label can be any detectable label, such as those described herein. The label will provide a means of detecting the binding of the candidate agent to TRPM7. In some binding assays, TRPM7 will be immobilized or covalently attached to a surface and contacted with a labeled candidate bioactive agent. In other assays, a library of candidate bioactive agents will be immobilized to a surface or covalently attached to a surface, e.g., biochip and contacted with a labeled TRPM7.

The present invention provides methods for blocking or reducing rat TRPM7 gene expression as well as methods for screening for a candidate bioactive agent capable of blocking or reducing TRPM7 gene expression and thus, TRPM7 activity. In some embodiments, TRPM7 gene expression will be blocked by anti-sense RNAs and DNAs, ribozymes or by targeting deoxyribonucleotide sequences complementary to the regulatory region of the target gene to form triple helical structures that prevent transcription of the target gene in target cells in the body. (See generally, Helene, Anticancer Drug Des., 6: 569-584, 1991; Helene, et al., Ann. N.Y. Acad. Sci., 660: 27-36, 1992; Maher, Bioassays, 14: 807-815, 1992).

Antisense nucleic acids (DNA, RNA, modified, analogues, and the like) can be made using any suitable method for producing a nucleic acid, such as the chemical synthesis and recombinant methods disclosed herein. In one embodiment, for example, antisense RNA molecules of the invention can be prepared by de novo chemical synthesis or by cloning. For example, an antisense RNA that hybridizes to rat TRPM7 mRNA can be made by inserting (ligating) a TRPM7 sequence in reverse orientation operably linked to a promoter in a vector (e.g., plasmid). Provided that the promoter and, preferably termination and polyadenylation signals, are properly positioned, the strand of the inserted sequence corresponding to the noncoding strand will be transcribed and act as an antisense oligonucleotide of the invention.

Ribozymes are enzymatic RNA molecules capable of catalyzing the specific cleavage of RNA. The mechanism of ribosome action involves sequence specific hybridization of the ribozyme molecule to complementary target RNA, followed by endonucleolytic cleavage. Within the scope of the invention are engineered hammerhead motif ribozyme molecules that specifically and efficiently catalyze endonucleolytic cleavage of rat TRPM7 sequences. Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site can be evaluated for predicted structural features such as secondary structure that can render the oligonucleotide sequence unsuitable. The suitability of candidate targets can also be evaluated by testing their accessibility to hybridization with complementary oligonucleotides, using ribonuclease protection assays.

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizable stretches of either purines or pyrimidines to be present on one strand of a duplex. Nucleotide sequences can be pyrimidine-based, which will result in TAT and CGC+ triplets across the three associated strands of the resulting triple helix. The pyrimidine-rich molecules provide base complementarily to a purine-rich region of a single strand of the duplex in a parallel orientation to that strand. In addition, nucleic acid molecules can be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation can be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

The anti-sense RNA and DNA molecules, ribozymes and triple helix molecules of the invention can be prepared by any method known in the art for the synthesis of RNA molecules. These include techniques for chemically synthesizing oligodeoxyribonucleotides that are well known in the art such a,s for example, solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors which contain suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

An alternative method of blocking or reducing TRPM7 gene expression is by sense suppression. Introduction of vectors in which a nucleic acid is configured in the sense orientation with respect to the promoter has been shown to be an effective means by which to block the transcription of target genes. Another means of inhibiting gene function is by creation of dominant negative mutations. In this approach, non-functional, mutant polypeptides of the present invention, which retain the ability to interact with wild-type subunits, are introduced.

Expression of TRPM7 can also be specifically suppressed by methods such as RNA interference (RNAi). A review of this technique is found in Science, 288: 1370-1372 (2000). Briefly, traditional methods of gene suppression, employing anti-sense RNA or DNA, operate by binding to the reverse sequence of a gene of interest such that binding interferes with subsequent cellular processes and therefore blocks synthesis of the corresponding protein. RNAi also operates on a post-translational level and is sequence specific, but suppresses gene expression far more efficiently. In RNA intereference methods, post-transcriptional gene silencing is brought about by a sequence-specific RNA degradation process which results in the rapid degradation of transcripts of sequence-related genes. Small nucleic acid molecules, such as short interfering nucleic acid (siNA), short interfering RNA (siRNA), double-stranded RNA (dsRNA), micro-RNA (mRNA), and short hairpin RNA (shRNA) molecules can all be used to modulate the expression of TRPM7 genes. Small nucleic acid molecules capable of suppressing TRPM7 through RNA intereference can be prepared by methods known in the art. See, for example, US Publication No. 2005/0124567 and Aarts et al., Cell 2003 115:863-877.

Accordingly, the present invention provides molecules capable of modulating e.g., blocking or reducing rat TRPM7 activity, as well as methods of screening for a candidate bioactive agent capable of modulating rat TRPM7 activity, such as anti-sense RNAs and DNAs, ribozymes, and other small nucleic acid molecules such as those described herein. All of these agents can be used as therapeutic agents for blocking the expression of certain TRPM7 genes in vivo. In some embodiments, they can be used to prevent TRPM7 gene transcription into mRNAs, to inhibit translation of TRPM7 mRNAs into proteins, and to block activities of preexisting TRPM7 proteins. Standard immunoassays, such as western blotting, ELISA, and the like, can be performed to confirm that the candidate bioactive agent has an effect on TRPM7 gene expression. Alternatively, TRPM7 expression can be determined by RT-PCR. Methods of performing RT-PCR are known in the art and are thus, not described herein. The effect of these molecules on TRPM7 channel activity can be assessed using a variety of assays described herein, including measuring current, measuring membrane potential, measuring ion flux, and measuring cell survival.

In some embodiments, the present invention provides methods for identifying molecules that modulate the divalent or monovalent cationic permeability of the TRPM7 channel.

Modulation of the monovalent cationic permeability of the TRPM7 channel can, for example, be determined by measuring the inward and outward currents in whole cell patch clamp assays or single-channel membrane patch assays in the presence and absence of the candidate bioactive agent. In an alternative embodiment, the modulation of monovalent cation activity can be monitored as a function of cation currents and/or membrane-potential of a cell comprising a TRPM7 channel. For example, the modulation of membrane potential can be detected with the use of a membrane potential-sensitive probe, such as bis-(1,3-dibutylbarbituric acid)trimethine oxonol (DiBAC4(3)) (Handbook of Fluorescent Probes and Research Chemicals, 9th ed. Molecular Probes). The use of a fluorescent membrane potential-sensitive probe allows rapid detection of change in membrane potential by monitoring change in fluorescence with the use of such methods as fluorescence microscopy, flow cytometry and fluorescence spectroscopy, including use of high through-put screening methods utilizing fluorescence detection (Alvarez-Barrientos, et al., “Applications of Flow Cytometry to Clinical Microbiology”, Clinical Microbiology Reviews, 13(2): 167-195, (2000)).

Modulation of the monovalent cationic permeability of the TRPM7 channel by a candidate agent can be determined by contacting a cell that expresses TRPM7 with a monovalent cation and a monovalent cation indicator that reacts with the monovalent cation to generate a signal. The intracellular levels of the monovalent cation can be measured by detecting the indicator signal in the presence and absence of a candidate bioactive agent. Additionally, the intracellular monovalent cation levels in cells that express TRPM7 with cells that do not express TRPM7 can be compared in the presence and absence of a candidate bioactive agent.

The monovalent cation indicator can be, for example, a sodium or potassium indicator. Examples of sodium indicators include SBFI, CoroNa Green, CoroNa Red, and Sodium Green (Handbook of Fluorescent Probes and Research Chemicals, 9th ed. Molecular Probes). Examples of potassium indicators include PBFI (Handbook of Fluorescent Probes and Research Chemicals, 9th ed. Molecular Probes).

The present invention provides methods for identifying molecules that modulate the divalent cationic permeability of the TRPM7 channel. The TRPM7 channel is permeable to the divalent cations, zinc, nickel, barium, cobalt, magnesium, manganese, strontium, cadmium, and calcium (Harteneck, Arch Pharmacol 2005 371:307-314). Modulation of the divalent cationic permeability of the TRPM7 channel can, for example, be determined by measuring the inward and outward currents in whole cell patch clamp assays or single-channel membrane patch assays in the presence and absence of the candidate bioactive agent. In an alternative embodiment, the modulation of divalent cation activity can be monitored as a function of cation currents and/or membrane-potential of a cell comprising a TRPM7 channel.

Modulation of the divalent cationic permeability of the TRPM7 channel by a candidate agent can be determined by contacting a cell that expresses TRPM7 with a divalent cation and a divalent cation indicator that reacts with the divalent cation to generate a signal. The intracellular levels of the divalent cation can be measured by detecting the indicator signal in the presence and absence of a candidate bioactive agent. Additionally, the intracellular divalent cation levels in cells that express TRPM7 with cells that do not express TRPM7 can be compared in the presence and absence of a candidate bioactive agent.

The divalent cation indicator can be, for example, a fluorescent magnesium indicator. Examples of magnesium indicators include furaptra (commercially available from Molecular Probes™, Invitrogen Detection Technologies).

Many forms of neurodegenerative disease are attributed to calcium ions. Excessive Ca²⁺ influx or release from intracellular stores can elevate Ca²⁺ loads to levels that exceed the capacity of Ca²⁺-regulator mechanisms (Aarts et al., Cell 2003 115:863-877). The methods of the present invention include methods of detecting Ca²⁺ flux through TRPM7 channels. The levels of intracellular Ca²⁺ levels are detectable, for example, using indicators specific for Ca²⁺. Indicators that are specific for Ca²⁺ include, but are not limited to, fura-2, indo-1, rhod-2, fura-4F, fura-5F, fura-6F and fura-FF, fluo-3, fluo-4, Oregon Green 488 BAPTA, Calcium Green, X-rhod-1 and fura-red (Handbook of Fluorescent Probes and Research Chemicals, 9th ed. Molecular Probes). Ca²⁺ loading can be determined by measuring Ca²⁺ accumulation in the cells. See, for example, Sattler et al., J. Neurochem, 1998 71, 2349-2364 and Aarts et al., Cell 2003 115:863-877.

Both the levels of monovalent and divalent cations into the cell can be measured either separately or simultaneously. For example, a Ca²⁺ specific indicator can be used to detect levels of Ca²⁺ and a monovalent cation specific indicator can be used to detect levels of monovalent cation. In some embodiments, the Ca²⁺ indicator and the monovalent cation specific indicator are chosen such that the signals from the indicators are capable of being detected simultaneously. For example, in some embodiments, both indicators will have a fluorescent signal but the excitation and/or emission spectra of both indicators will be distinct such that the signal from each indicator can be detected at the same time.

In yet another embodiment, both the levels of divalent or monovalent cations and the change in membrane potential can be measured simultaneously. In this embodiment a Ca²⁺ specific indicator can be used to detect levels of Ca²⁺ and a membrane potential sensitive probe can be used to detect changes in the membrane potential. The Ca²⁺ indicator and the membrane potential sensitive probe can be chosen such that the signals from the indictors and probes are capable of being detected simultaneously. For example, in some embodiments, both the indicator and probe will have a fluorescent signal but the excitation and/or emission spectra of both indicators will be distinct such that the signal from each indicator can be detected at the same time.

In some embodiments of the present invention, before modulation of the TRPM7 channel can be measured, TRPM7 must be activated. TRPM7 channels are activated by millimolar levels of MgATP levels (Nadler et al., Nature 2001 411:590-595)

In some embodiments, the ion permeability of TRPM7 is measured in intact cells, e.g., HEK-293 cells, that are transformed with a vector comprising nucleic acid encoding TRPM7 and an inducible promoter operably linked thereto. After inducement of the promoter, the TRPM7 polypeptides are produced and form a TRPM7 channel. Endogenous levels of intracellular ions can be measured prior to inducement and then compared to the levels of intracellular ions measured subsequent to inducement. In one embodiment, fluorescent molecules can be used to detect intracellular monovalent and divalent cation levels.

In certain embodiments, the candidate bioactive agents can, for example, open TRPM7 channels in a variety of cells such as cells of the nervous systems of vertebrates. In a preferred embodiment, the candidate bioactive agents will close, e.g., inhibit, TRPM7 channels in a variety of cells such as cells of the nervous. Preferred candidate bioactive agents will close or inhibit TRPM7 channels. The closing or inhibition of the TRPM7 channels can, for example, prevent or significantly decrease neuronal cell death following ischemic injury.

The present provides methods for identifying candidate bioactive agents that modulate expression levels of TRPM7 within cells. Candidate agents can be used that wholly or partially suppress or enhance the expression of TRPM7 within cells, thereby altering the cellular phenotype. Examples of these candidate agents include antisense cDNAs and DNAs, regulatory binding proteins and/or nucleic acids, as well as any of the other candidate bioactive agents herein described that modulate transcription or translation of nucleic acids encoding TRPM7.

The present invention also provides antibodies that specifically bind to unique epitopes on the TRPM7 polypeptide, e.g., unique epitopes of the protein. Such antibodies can be assayed not only for binding to TRPM7 but also for their ability to modulate TRPM7 monovalent cation permeability. The anti-TRPM7 polypeptide antibodies can comprise polyclonal or monoclonal antibodies. The anti-TRPM7 polypeptide antibodies can further comprise monovalent antibodies. The anti-TRPM7 polypeptide antibodies can further comprise humanized antibodies or human antibodies. The anti-TRPM7 polypeptide antibodies can further comprise heteroconjugate antibodies. Heteroconjugate antibodies are composed of two covalently joined antibodies.

A particularly useful assay for use in the present invention measures the effect that a compound of interest has on cells expressing TRPM7 that have been denied oxygen and glucose. By measuring cell survival or cell death after the denial of oxygen and glucose and comparing the amount of cell survival in a control cell sample versus the amount of cell survival in a cell sample treated with a test compound, it can be determined whether the test compound is a modulator of TRPM7 activity and of ischemic death. Assays for measuring cell survival are known in the art and include, for example, assays for measuring lactate dehydrogenase which is released from dying cells and assays for measuring ATP in living cells. A preferred candidate bioactive agent will rescue cells that have been denied oxygen and glucose. If desired, further tests can be performed to confirm that the compound had an effect on TRPM7 gene expression or biological activity of the protein. Standard immunoassays can be used, such as western blotting, ELISA and the like. For measurement of mRNA, amplification, e.g., using PCR, LCR, or hybridization assays, e.g., northern hybridization, RNase protection, dot blotting, are preferred. The level of protein or mRNA can be detected, for example, using directly or indirectly labeled detection agents, e.g., fluorescently or radioactively labeled nucleic acids, radioactively or enzymatically labeled antibodies, and the like, as described herein. After a compound is determined to have an effect on rat TRPM7 activity or/and gene or protein expression or/and cell survival, the compound can be used in an animal model, in particular a rat model, for ischemic injury, including, for example, stroke.

Preferably, the TRPM7 used in these assays will have at least 99% identity to the amino acid sequence as set forth in SEQ ID NO:2. Preferably rat cells and cell lines are used.

A. Candidate Bioactive Agents

The term “modulator”, “candidate substance”, “candidate bioactive agent”, “drug candidate”, “agent” or grammatical equivalents as used herein describes any molecule, e.g., protein, oligopeptide, small organic molecule, polysaccharide, polynucleotide or oligonucleotide (e.g., antisense, siRNA), to be tested for bioactive agents that are capable of directly or indirectly altering the activity of a target gene, protein, or cell. Accordingly, the term “candidate bioactive agent” as used herein describes any molecule that binds to TRPM7, modulates the activity of a TRPM7 ion channel, or alters the expression of TRPM7 within cells. Candidate agents may be bioactive agents that are known or suspected to bind to ion channel proteins or known to modulate the activity of ion channel proteins, or alter the expression of ion channel proteins within cells. Candidate agents can also be mimics of bioactive agents that are known or suspected to bind to ion channel proteins or known to modulate the activity of ion channel proteins, or alter the expression of ion channel proteins within cells. In a particularly preferred method, the candidate agents induce a response, or maintain such a response as indicated, for example, reduction of neuronal cell death following ischemic injury.

Candidate agents encompass numerous chemical classes, though typically they are organic molecules. Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents can be subjected to directed or random chemical modifications, such as acylation, alkylation, esterification, amidification to produce structural analogs.

B. Combinatorial Chemical Libraries

The invention provides methods for identifying/screening for modulators (e.g., inhibitors, activators) of rat TRPM7 activity. In practicing the screening methods of the invention, a candidate compound is provided. Combinatorial chemical libraries are one means to assist in the generation of new chemical compound leads for, e.g., compounds that inhibit a rat TRPM7 activity. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, the systematic, combinatorial mixing of 100 interchangeable chemical building blocks results in the theoretical synthesis of 100 million tetrameric compounds or 10 billion pentameric compounds. (See, e.g., Gallop et al., J. Med. Chem. 1994, 37: 1233-1250). Preparation and screening of combinatorial chemical libraries are well known to those of skill in the art, (see, e.g., U.S. Pat. Nos. 6,004,617; 5,985,356). Such combinatorial chemical libraries include, but are not limited to, peptide libraries. (see, e.g., U.S. Pat. No. 5,010,175; Furka, Int. J. Pept. Prot. Res. 1991, 37: 487-493; Houghton et al., Nature 1991, 354: 84-88). Other chemistries for generating chemical diversity libraries include, but are not limited to: peptoids (see, e.g., WO 91/19735), encoded peptides (see, e.g., WO 93/20242), random bio-oligomers (see, e.g., WO 92/00091), benzodiazepines (see, e.g., U.S. Pat. No. 5,288,514), diversomers such as hydantoins, benzodiazepines and dipeptides (see, e.g., Hobbs, Proc. Nat. Acad. Sci. USA 1993, 90: 6909-6913), vinylogous polypeptides (see, e.g., Hagihara, J. Amer. Chem. Soc. 1992, 114: 6568), non-peptidal peptidomimetics with a Beta-D-Glucose scaffolding (see, e.g., Hirschmann, J. Amer. Chem. Soc. 1992, 114: 9217-9218), analogous organic syntheses of small compound libraries (see, e.g., Chen, J. Amer. Chem. Soc. 1994, 116: 2661), oligocarbamates (see, e.g., Cho, Science 1993, 261:1303), and/or peptidyl phosphonates (see, e.g., Campbell, J. Org. Chem. 1994, 59: 658). See also (Gordon, J. Med. Chem. 1994, 37: 1385); for nucleic acid libraries, peptide nucleic acid libraries, (see, e.g., U.S. Pat. No. 5,539,083); for antibody libraries, (see, e.g., Vaughn, Nature Biotechnology 1996, 14: 309-314); for carbohydrate libraries, (see, e.g., Liang et al., Science 1996, 274: 1520-1522, U.S. Pat. No. 5,593,853); for small organic molecule libraries, (see, e.g., for isoprenoids U.S. Pat. No. 5,569,588); for thiazolidinones and metathiazanones, (U.S. Pat. No. 5,549,974); for pyrrolidines, (U.S. Pat. Nos. 5,525,735) and 5,519,134; for morpholino compounds, (U.S. Pat. No. 5,506,337); for benzodiazepines (U.S. Pat. No. 5,288,514).

Devices for the preparation of combinatorial libraries are commercially available (see, e.g., U.S. Pat. Nos. 6,045,755; 5,792,431; 357 MPS, 390 MPS), (Advanced Chem Tech, Louisville Ky., Symphony, Rainin, Woburn, Mass., 433A Applied Biosystems, Foster City, Calif., 9050 Plus, Millipore, Bedford, Mass.). A number of robotic systems have also been developed for solution phase chemistries. These systems include automated workstations, e.g., like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) that mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., and the like).

The compounds tested as modulators of rat TRPM7 genes or gene products can be any small organic molecule, or a biological entity, such as a protein, e.g., an antibody or peptide, a sugar, a nucleic acid, e.g., an antisense oligonucleotide or RNAi, or a ribozyme, or a lipid. Alternatively, modulators can be genetically altered versions of a rat TRPM7 protein. Typically, test compounds will be small organic molecules, peptides, lipids, and lipid analogs.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, that are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one embodiment, high throughput screening methods involve providing a combinatorial small organic molecule or peptide library containing a large number of potential therapeutic compounds (potential modulator or ligand compounds). Such “combinatorial chemical libraries” or “ligand libraries” (as described above) are then screened in one or more assays, as described herein, to identify those library members (particular chemical species or subclasses) that display a desired characteristic activity. The compounds thus identified can serve as conventional “lead compounds” or can themselves be used as potential or actual therapeutics.

C. Arrays or “Biochips”

The invention provides methods for identifying/screening for modulators (e.g., inhibitors, activators) of rat TRPM7 activity, using arrays. Potential modulators, including small molecules, nucleic acids, polypeptides (including antibodies) can be immobilized to arrays. Nucleic acids or polypeptides of the invention can be immobilized to or applied to an array. Arrays can be used to screen for or monitor libraries of compositions (e.g., small molecules, antibodies, nucleic acids, and the like) for their ability to bind to or modulate the activity of a nucleic acid or a polypeptide of the invention. For example, in one aspect of the invention, a monitored parameter is transcript expression of a gene comprising a nucleic acid of the invention. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Alternatively, arrays comprising genomic nucleic acid can also be used to determine the genotype of a newly engineered strain made by the methods of the invention. Polypeptide arrays can be used to simultaneously quantify a plurality of proteins. Small molecule arrays can be used to simultaneously analyze a plurality of TRPM7 modulating or binding activities.

The present invention can be practiced with any known “array,” also referred to as a “microarray” or “nucleic acid array” or “polypeptide array” or “antibody array” or “biochip,” or variation thereof. Arrays are generically a plurality of “spots” or “target elements,” each target element comprising a defined amount of one or more biological molecules, e.g., oligonucleotides, immobilized onto a defined area of a substrate surface for specific binding to a sample molecule, e.g., mRNA transcripts. In practicing the methods of the invention, any known array and/or method of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; (see also, e.g., WO 99/51773; WO 99/09217; WO 97/46313; WO 96/17958; see also, e.g., Johnston, Curr. Biol. 8, 1998; Schummer, Biotechniques 1997, 23: 1087-1092; Kern, Biotechniques 1997, 23: 120-124; Solinas-Toldo, Genes, 1997, 20: 399-407; Bowtell, Nature Genetics Supp. 1999. 21: 25-32). See also published U.S. patent applications Nos. 20010018642; 20010019827; 20010016322; 20010014449; 20010014448; 20010012537; 20010008765.

The terms “array” or “microarray” or “biochip” or “chip” as used herein is a plurality of target elements, each target element comprising a defined amount of one or more polypeptides (including antibodies) or nucleic acids immobilized onto a defined area of a substrate surface.

D. Solid State and Soluble High Throughput Assays

In certain embodiments, the invention provide soluble assays using molecules such as a domain such as ligand binding domain, an active site, and the like; a domain that is covalently linked to a heterologous protein to create a chimeric molecule; rat TRPM7; a cell or tissue expressing rat TRPM7, either naturally occurring or recombinant. In another embodiment, the invention provides solid phase based in vitro assays in a high throughput format, where the domain, chimeric molecule, rat TRPM7, or cell or tissue expressing rat TRPM7 is attached to a solid phase substrate.

In exemplary high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-1500 different compounds. It is possible to assay several different plates per day; assay screens for up to about 6,000-20,000 different compounds is possible using the integrated systems of the invention.

The molecule of interest can be bound to the solid state component, directly or indirectly, via covalent or non covalent linkage, e.g., via a tag. The tag can be any of a variety of components. In general, a molecule that binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, and the like) Antibodies to molecules with natural binders such as biotin are also widely available and appropriate tag binders; see, SIGMA Immunochemicals 1998 catalogue SIGMA, St. Louis Mo.

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody that recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott et al., The Adhesion Molecule Facts Book I , 1993. Similarly, toxins and venoms, viral epitopes, hormones (e.g., opiates, steroids, and the like), intracellular receptors (e.g. that mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages.

Tag binders can be fixed to solid substrates using any of a variety of methods currently available. Solid substrates are commonly derivatized or functionalized by exposing all or a portion of the substrate to a chemical reagent that fixes a chemical group to the surface that is reactive with a portion of the tag binder. For example, groups that are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. (See, e.g., Merrifield, J. Am. Chem. Soc. 1963 85: 2149-2154(describing solid phase synthesis of, e.g., peptides); Geysen et al., J. Immun. Meth. 1987 102: 259-274 (describing synthesis of solid phase components on pins); Frank et al., Tetrahedron 1988, 44: 6031-6040, (describing synthesis of various peptide sequences on cellulose disks); Fodor et al., Science, 1991, 251: 767-777; Sheldon et al., Clinical Chemistry 1993, 39: 718-719; and Kozal et al., Nature Medicine 1996, 7: 753-759 (all describing arrays of biopolymers fixed to solid substrates). Non-chemical approaches for fixing tag binders to substrates include other common methods, such as heat, cross-linking by UV radiation, and the like.

E. Computer-Based Assays

Yet another assay for compounds that modulate rat TRPM7 activity involves computer assisted drug design, in which a computer system is used to generate a three-dimensional structure of rat TRPM7 based on the structural information encoded by the amino acid sequence. The input amino acid sequence interacts directly and actively with a preestablished algorithm in a computer program to yield secondary, tertiary, and quaternary structural models of the protein. The models of the protein structure are then examined to identify regions of the structure that have the ability to bind, e.g., ligands. These regions are then used to identify ligands that bind to the protein.

The three-dimensional structural model of the protein is generated by entering rat TRPM7 amino acid sequences of at least 10 amino acid residues or corresponding nucleic acid sequences encoding a rat TRPM7 polypeptide into the computer system. The amino acid sequence of the polypeptide or the nucleic acid encoding the polypeptide is selected from the group consisting of the sequences provided herein, and conservatively modified versions thereof. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art. The three-dimensional structural model of the protein can be saved to a computer readable form and be used for further analysis (e.g., identifying potential ligand binding regions of the protein and screening for mutations, alleles and interspecies homologs of the gene).

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential ligand binding regions are identified by the computer system. Three-dimensional structures for potential ligands are generated by entering amino acid or nucleotide sequences or chemical formulas of compounds, as described above. The three-dimensional structure of the potential ligand is then compared to that of the rat TRPM7 protein to identify ligands that bind to rat TRPM7. Binding affinity between the protein and ligands is determined using energy terms to determine which ligands have an enhanced probability of binding to the protein. The results, such as three-dimensional structures for potential ligands and binding affinity of ligands, can also be saved to a computer readable form and can be used for further analysis (e.g., generating a three dimensional model of mutated proteins having an altered binding affinity for a ligand).

7. Transgenic and “Knock-Out” Animals

The invention provides transgenic rats comprising a nucleic acid, a polypeptide, an expression cassette or vector or a transfected or transformed cell of the invention. A “transgenic rat” is a rat having cells that contain DNA that has been artificially inserted into a cell, which DNA becomes part of the genome of the animal that develops from that cell.

These rats can be used, e.g., as in vivo models to screen for agents that modulate rat TRPM7 activity in vivo.

In one aspect, the inserted transgenic sequence is a sequence of the invention designed such that it does not express a functional rat TRPM7 polypeptide. The defect can be designed to be on the transcriptional, translational and/or the protein level.

The coding sequences for the polypeptides, e.g., rat TRPM7 polypeptides, to be expressed in the transgenic non-human animals can be designed to be constitutive, or, under the control of tissue-specific, developmental-specific or inducible transcriptional regulatory factors. Transgenic rats can be designed and generated using any method known in the art; (see, e.g., U.S. Pat. Nos. 6,211,428; 6,187,992; 6,156,952; 6,118,044; 6,111,166; 6,107,541; 5,959,171; 5,922,854; 5,892,070; 5,880,327; 5,891,698; 5,639,940; 5,573,933; 5,387,742; 5,087,571, describing making and using transformed cells and eggs and transgenic mice, rats, rabbits, sheep, pigs and cows). One exemplary method to produce genetically altered rats is to genetically modify embryonic stem cells. The modified cells are injected into the blastocoel of a blastocyst. This is then grown in the uterus of a pseudopregnant female. In order to readily detect chimeric progeny, the blastocysts can be obtained from a different parental line than the embryonic stem cells. For example, the blastocysts and embryonic stem cells can be derived from parental lines with different hair color or other readily observable phenotype. The resulting chimeric rats can be bred in order to obtain non-chimeric rats that have received the modified genes through germ-line transmission. Techniques for the introduction of embryonic stem cells into blastocysts and the resulting generation of transgenic animals are well known.

Because cells contain more than one copy of a gene, the cell lines obtained from a first round of targeting are likely to be heterozygous for the targeted allele. Homozygosity, in which both alleles are modified, can be achieved in a number of ways. In one approach, a number of cells in which one copy has been modified are grown. They are then subjected to another round of targeting using a different selectable marker. Alternatively, homozygotes can be obtained by breeding animals heterozygous for the modified allele, according to traditional Mendelian genetics. In some situations, it may be desirable to have two different modified alleles. This can be achieved by successive rounds of gene targeting or by breeding heterozygotes, each of which carries one of the desired modified alleles. See, e.g., (U.S. Pat. No. 5,789,215).

A variety of methods are available for the production of transgenic rats associated with this invention. DNA can be injected into the pronucleus of a fertilized egg before fusion of the male and female pronuclei, or injected into the nucleus of an embryonic cell (e.g., the nucleus of a two-cell embryo) following the initiation of cell division (Brinster et al., Proc. Nat. Acad. Sci. 1985, 82: 4438-4442). Embryos can be infected with viruses, especially retroviruses, modified to carry inorganic-ion receptor nucleotide sequences of the invention.

Pluripotent stem cells derived from the inner cell mass of the embryo and stabilized in culture can be manipulated in culture to incorporate nucleotide sequences of the invention. A transgenic rats can be produced from such cells through implantation into a blastocyst that is implanted into a foster mother and allowed to come to term.

The procedures for manipulation of the rodent embryo and for microinjection of DNA into the pronucleus of the zygote are well known to those of ordinary skill in the art (Hogan et al., supra).

Methods for the culturing of embryonic stem (ES) cells and the subsequent production of transgenic animals by the introduction of DNA into ES cells using methods such as electroporation, calcium phosphate/DNA precipitation and direct injection also are well known to those of ordinary skill in the art (E. J. Robertson, Teratocarcinomas and Embryonic Stem Cells, A Practical Approach, 1987).

In cases involving random gene integration, a clone containing the sequence(s) of the invention is co-transfected with a gene encoding resistance. Alternatively, the gene encoding neomycin resistance is physically linked to the sequence(s) of the invention. Transfection and isolation of desired clones are carried out by any one of several methods well known to those of ordinary skill in the art (E. J. Robertson, supra).

DNA molecules introduced into ES cells can also be integrated into the chromosome through the process of homologous recombination (Capecchi, Science 1989, 244: 1288-1292). Methods for positive selection of the recombination event (i.e., neo resistance) and dual positive-negative selection (i.e., neo resistance and gancyclovir resistance) and the subsequent identification of the desired clones by PCR have been described by (Capecchi, supra and Joyner et al. Nature 1989, 338: 153-156), the teachings of which are incorporated herein in their entirety including any drawings. The final phase of the procedure is to inject targeted ES cells into blastocysts and to transfer the blastocysts into pseudopregnant females. The resulting chimeric animals are bred and the offspring are analyzed by Southern blotting to identify individuals that carry the transgene. Procedures for the production of non-rodent mammals and other animals have been discussed by others (Houdebine et al., supra; Pursel et al., Science 1989, 244: 1281-1288; and Simms et al., Bio/Technology 1988, 6: 179-183).

A. Rat TRPM7 Functional Knock-Outs

The invention provides rats that do not express their endogenous rat TRPM7 polypeptides, or, express their endogenous rat TRPM7 polypeptides at lower than wild type levels (thus, while not completely “knocked out” their rat TRPM7 activity is functionally “knocked out”). The invention also provides “knock-out animals” and methods for making and using them. For example, in one aspect, the transgenic or modified rats of the invention comprise a “knock-out rats,” engineered not to express an endogenous gene, e.g., an endogenous rat TRPM7 gene, which is replaced with a gene expressing a polypeptide of the invention, or, a fusion protein comprising a polypeptide of the invention. Thus, in one aspect, the inserted transgenic sequence is a sequence of the invention designed such that it does not express a functional rat TRPM7 polypeptide. The defect can be designed to be on the transcriptional, translational and/or the protein level. Because the endogenous rat TRPM7 gene has been “knocked out,” only the inserted polypeptide of the invention is expressed.

A “knock-out animal” is a specific type of transgenic animal having cells that contain DNA containing an alteration in the nucleic acid sequence that reduces the biological activity of the polypeptide normally encoded therefrom by at least 80% compared to the unaltered gene. The alteration can be an insertion, deletion, frameshift mutation, missense mutation, introduction of stop codons, mutation of critical amino acid residue, removal of an intron junction, and the like. Preferably, the alteration is an insertion or deletion, or is a frameshift mutation that creates a stop codon. Typically, the disruption of specific endogenous genes can be accomplished by deleting some portion of the gene or replacing it with other sequences to generate a null allele. Cross-breeding mammals having the null allele generates a homozygous mammals lacking an active copy of the gene.

Variations on this basic technique also exist and are well known in the art. For example, a “knock-in” construct refers to the same basic arrangement of a nucleic acid encoding a 5′ genomic locus fragment linked to nucleic acid encoding a positive selectable marker, which in turn is linked to a nucleic acid encoding a 3′ genomic locus fragment, but which differs in that none of the coding sequence is omitted and thus the 5′ and the 3′ genomic fragments used were initially contiguous before being disrupted by the introduction of the nucleic acid encoding the positive selectable marker gene. This “knock-in” type of construct is thus very useful for the construction of mutant transgenic animals when only a limited region of the genomic locus of the gene to be mutated, such as a single exon, is available for cloning and genetic manipulation. Alternatively, the “knock-in” construct can be used to specifically eliminate a single functional domain of the targeted gene, resulting in a transgenic animal that expresses a polypeptide of the targeted gene that is defective in one function, while retaining the function of other domains of the encoded polypeptide. This type of “knock-in” mutant frequently has the characteristic of a so-called “dominant negative” mutant because, especially in the case of proteins that homomultimerize, it can specifically block the action of the polypeptide product of the wild-type gene from which it was derived.

Each knock-out construct to be inserted into the cell must first be in the linear form. Therefore, if the knock-out construct has been inserted into a vector, linearization is accomplished by digesting the DNA with a suitable restriction endonuclease selected to cut only within the vector sequence and not within the knock-out construct sequence. For insertion, the knock-out construct is added to the ES cells under appropriate conditions for the insertion method chosen, as is known to the skilled artisan. Where more than one construct is to be introduced into the ES cell, each knock-out construct can be introduced simultaneously or one at a time.

After suitable ES cells containing the knock-out construct in the proper location have been identified by the selection techniques outlined above, the cells can be inserted into an embryo. Insertion can be accomplished in a variety of ways known to the skilled artisan, however a preferred method is by microinjection. For microinjection, about 10-30 cells are collected into a micropipette and injected into embryos that are at the proper stage of development to permit integration of the foreign ES cell containing the knock-out construct into the developing embryo. For instance, the transformed ES cells can be microinjected into blastocytes. The suitable stage of development for the embryo used for insertion of ES cells is very species dependent. The embryos are obtained by perfusing the uterus of pregnant females. Suitable methods for accomplishing this are known to the skilled artisan. After the ES cell has been introduced into the embryo, the embryo can be implanted into the uterus of a pseudopregnant foster mother for gestation as described above.

Yet other methods of making knock-out or disruption transgenic animals are also generally known. See, for example, (Hogan B. et al., Manipulating The Mouse Embryo, 2ND EDITION, Cold Spring Harbor Press 1994). Recombinase dependent knock-outs can also be generated, e.g. by homologous recombination to insert target sequences, such that tissue specific and/or temporal control of inactivation of a target gene can be controlled by recombinase sequences (described infra).

Animals containing more than one knock-out construct and/or more than one transgene expression construct can be prepared any of several ways. The preferred manner of preparation is to generate a series of mammals, each containing one of the desired transgenic phenotypes. Such animals are bred together through a series of crosses, backcrosses and selections, to ultimately generate a single animal containing all desired knock-out constructs and/or expression constructs, where the animal is otherwise congenic (genetically identical) to the wild type except for the presence of the knock-out construct(s) and/or transgene(s).

The functional rat TRPM7 “knock-out” rats of the invention are of several types. Some rats of the invention that are functional rat TRPM7 “knock-outs” express sufficient levels of a rat TRPM7 inhibitory nucleic acid, e.g., antisense sequences or ribozymes of the invention, to decrease the levels or knock-out the expression of functional polypeptide. Some rats of the invention that are functional rat TRPM7 “knock-outs” express sufficient levels of a rat TRPM7 dominant negative polypeptide such that the effective amount of free endogenous active rat TRPM7 is decreased. Some rats of the invention that are rat TRPM7 “knock-outs” express sufficient levels of an antibody of the invention, e.g., a rat TRPM7 antibody, such that the effective amount of free endogenous active rat TRPM7 is decreased. Some non-human animals of the invention that are functional rat TRPM7 “knock-outs” are “conventional” knock-outs in that their endogenous rat TRPM7 gene has been disrupted or mutated.

8. Kits for Identifying Modulators of TRPM7 Activity

Also provided are kits that allow for the identification of modulators of TRPM7 activity. In one embodiment, such kits provide the materials necessary to assess the activity of rat TRPM7 in vitro. In one embodiment, this kit contains aliquots of isolated rat TRPM7 and cultured cells. In another embodiment, the kit contains cell lines that express either wildtype or mutant rat TRPM7. In yet another embodiment, additional components in some of these kits may include instructions for carrying out the assay. In other embodiments, reaction vessels and auxiliary reagents such as chromogens, buffers, media, enzymes, and the like may also be included in the kits.

### EXAMPLES

### Example 1

### The Cloning of Rat TRPM7 Gene

Rat cDNA was reverse transcripted from rat brain RNA and used as a template for PCR of the rat TRPM7 gene. PCR primers were designed based on virtual cDNA sequence of rat TRPM7. Two pieces of PCR fragments covering the open reading frame and some UTR region, a 4355 bp and 4566 bp region, were obtained and cloned into a PCRII TOPO vector. They were put together as one piece of a 6982 bp insert in the PCRII vector. The sequence was confirmed and aligned with human, mouse and rat genomic sequence.

The primers were designed as follows:

| | |
|---|---|
| rTRPM7F38 | |
| 5′AGCTGGTCGCACAATTATGAAAG3′ | (SEQ ID NO:10) |
| rTRPM7R4939 | |
| 5′ATACTCTGCGACAGCCTCATCA3′ | (SEQ ID NO:11) |
| rTRPM7F2456 | |
| 5′TGCTGTTGTCTGATATGTGGATG3′ | (SEQ ID NO:12) |
| rTRPM7R7022 | |
| 5′CACCTTTTCTCTCCACATCAACTT3′ | (SEQ ID NO:13) |

### Example 2

### Sequence Alignment of Rat, Mouse and Human TRPM7 Nucleic Acid

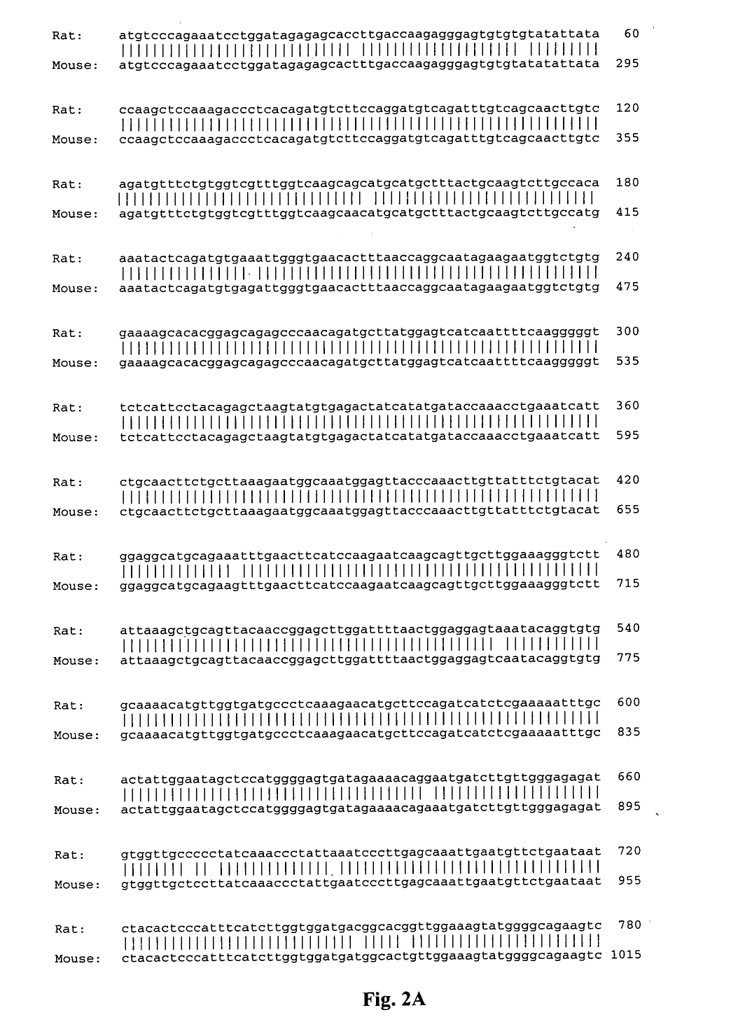

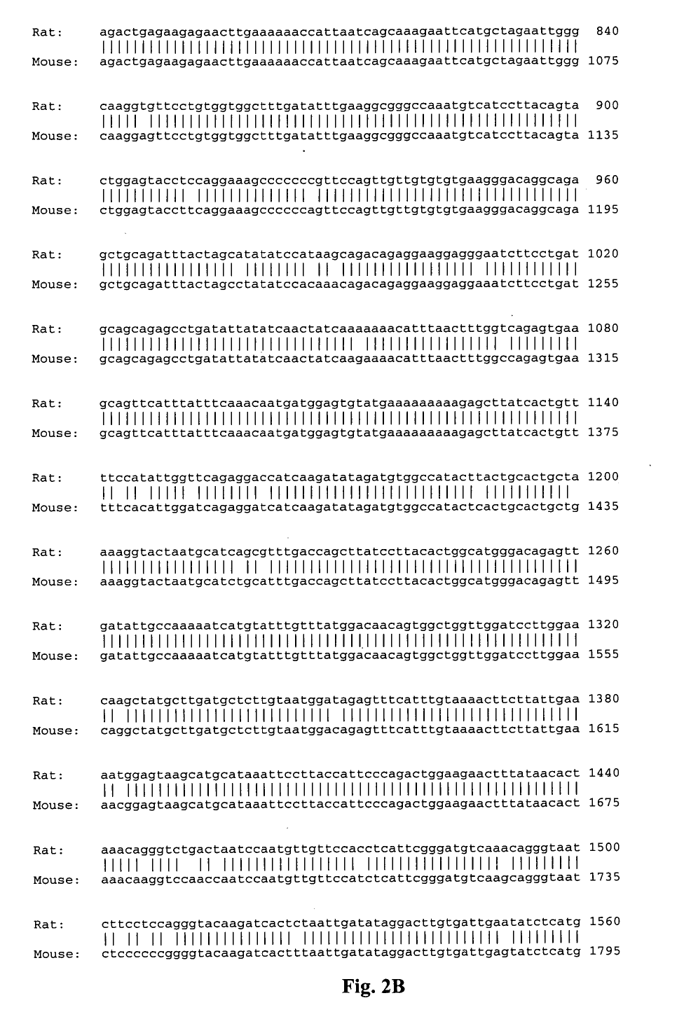

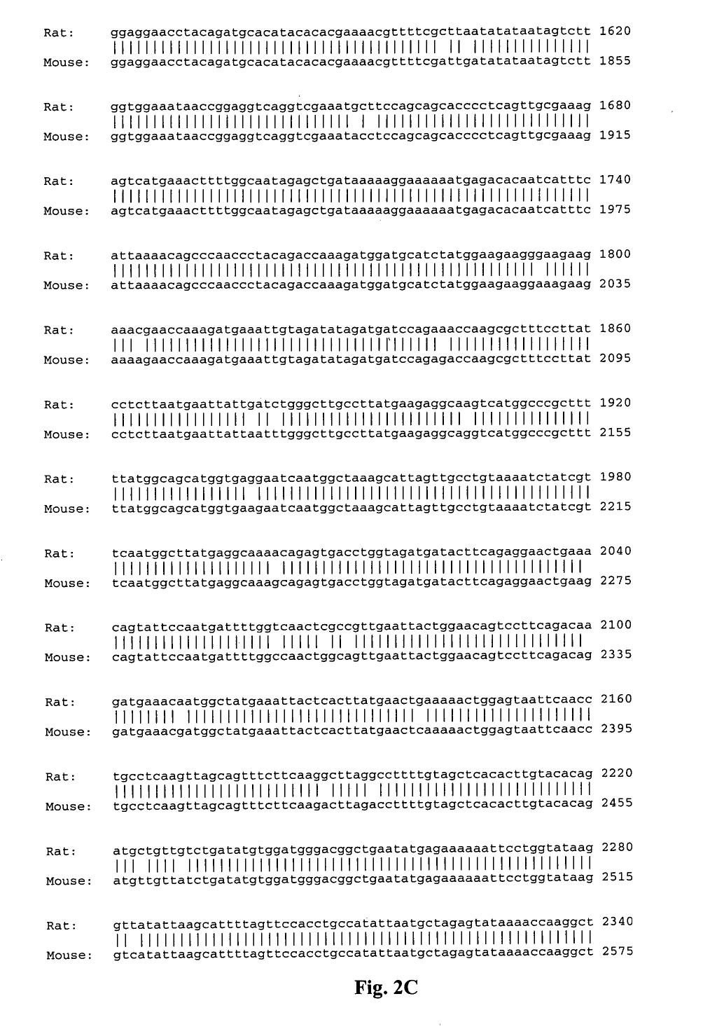

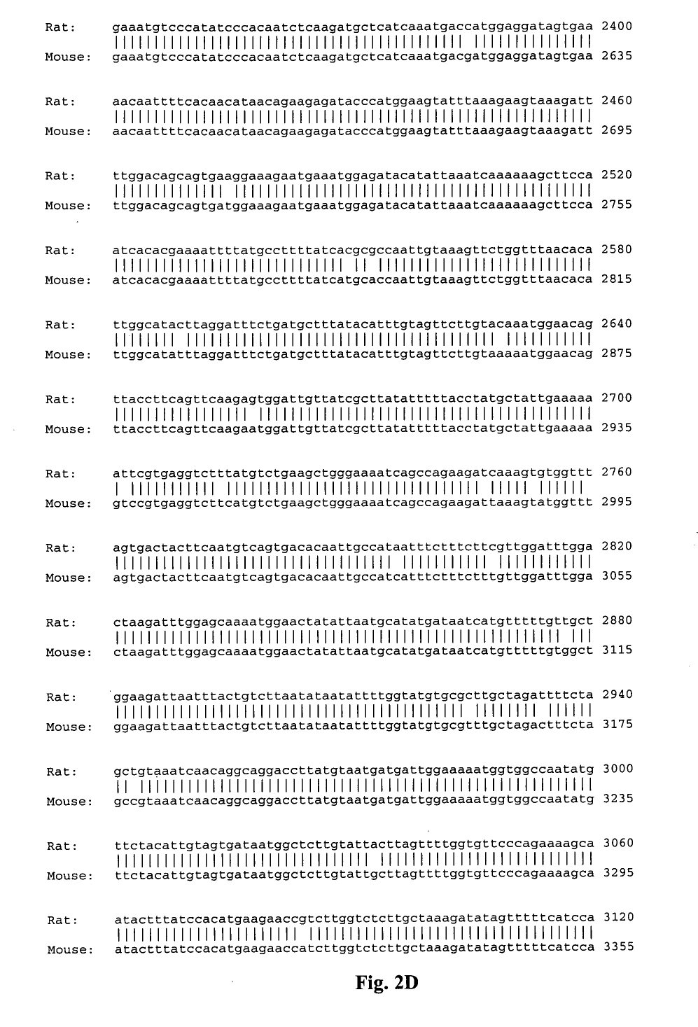

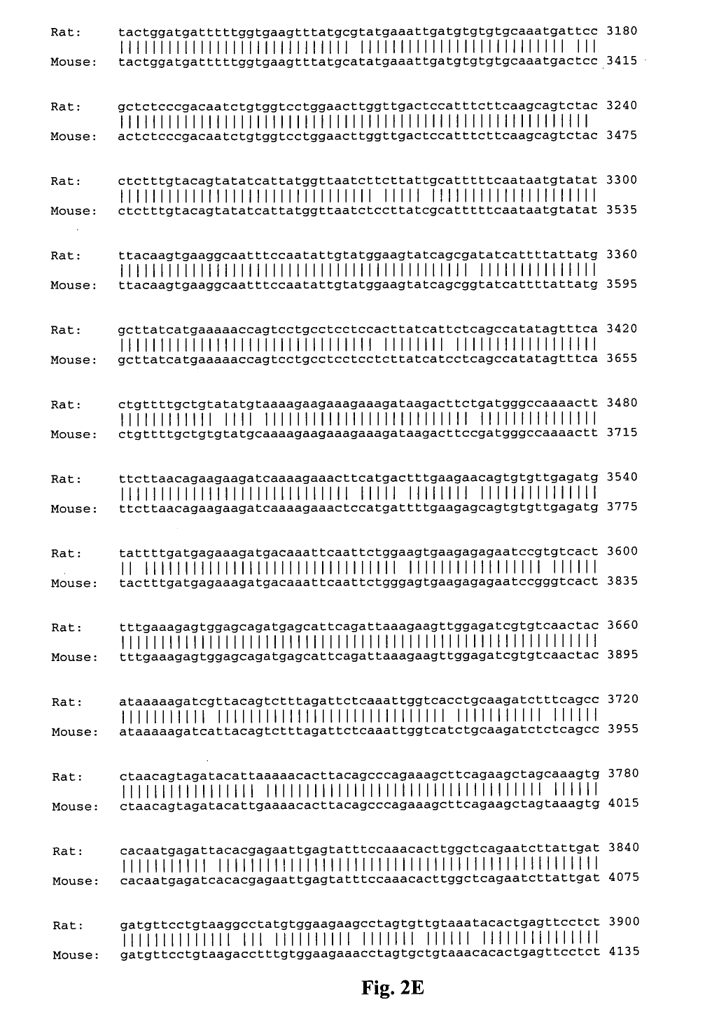

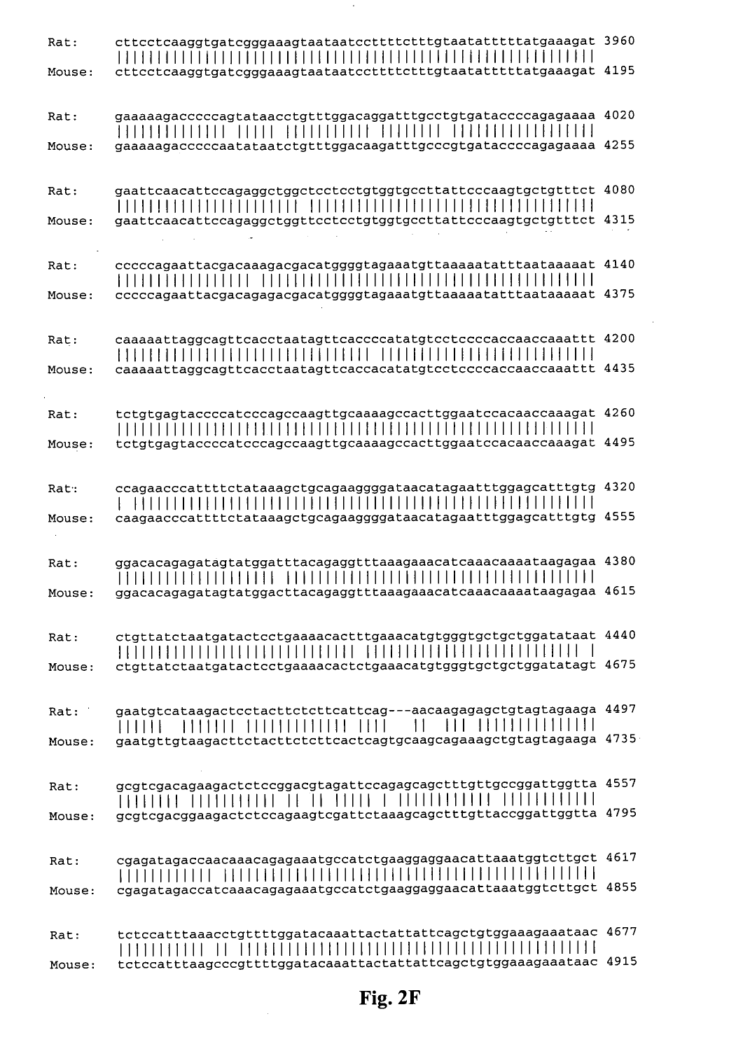

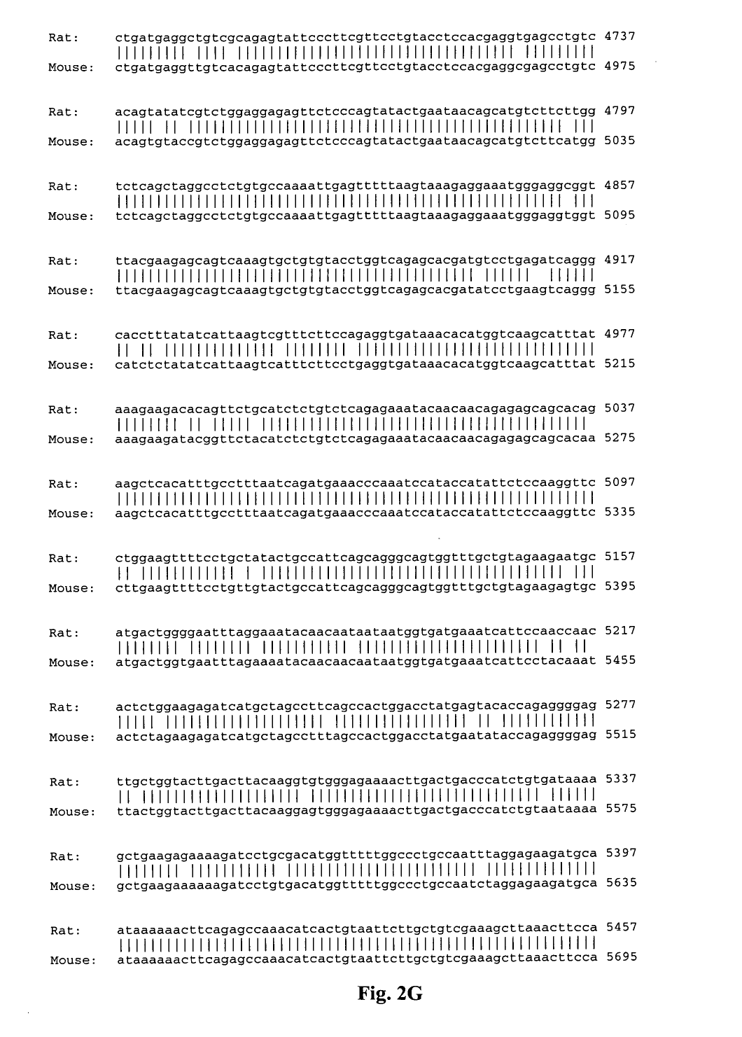

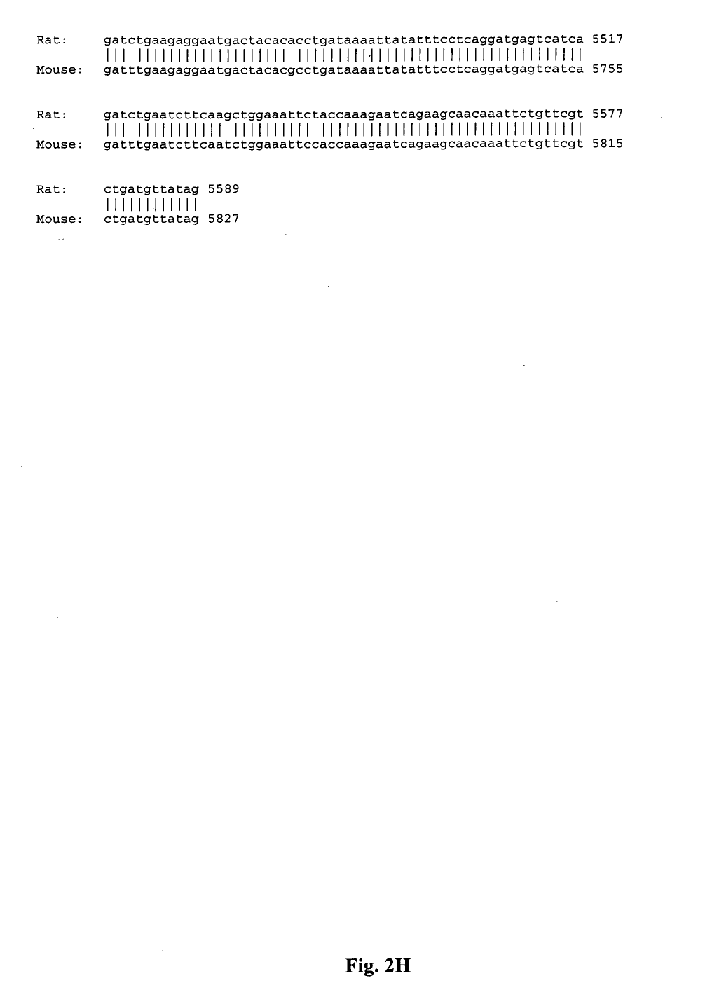

Rat TRPM7 open reading frame (SEQ ID NO:1) was compared with mouse TRPM7 (NM_{—}021450.1) (SEQ ID NO:14). The results are shown in FIG. 2.

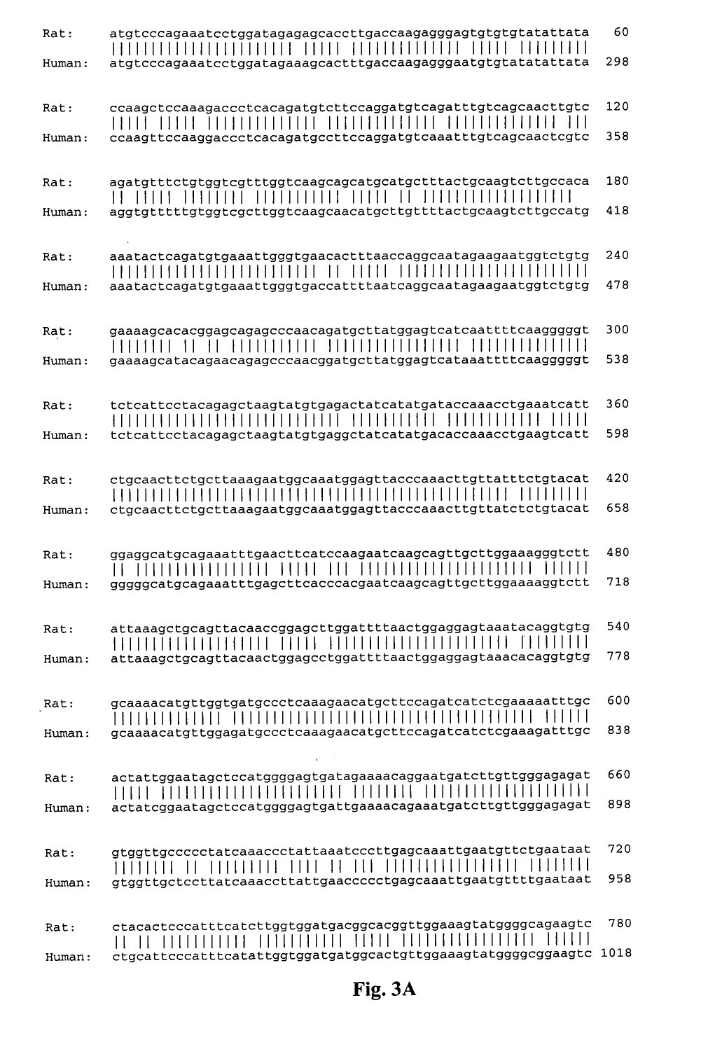

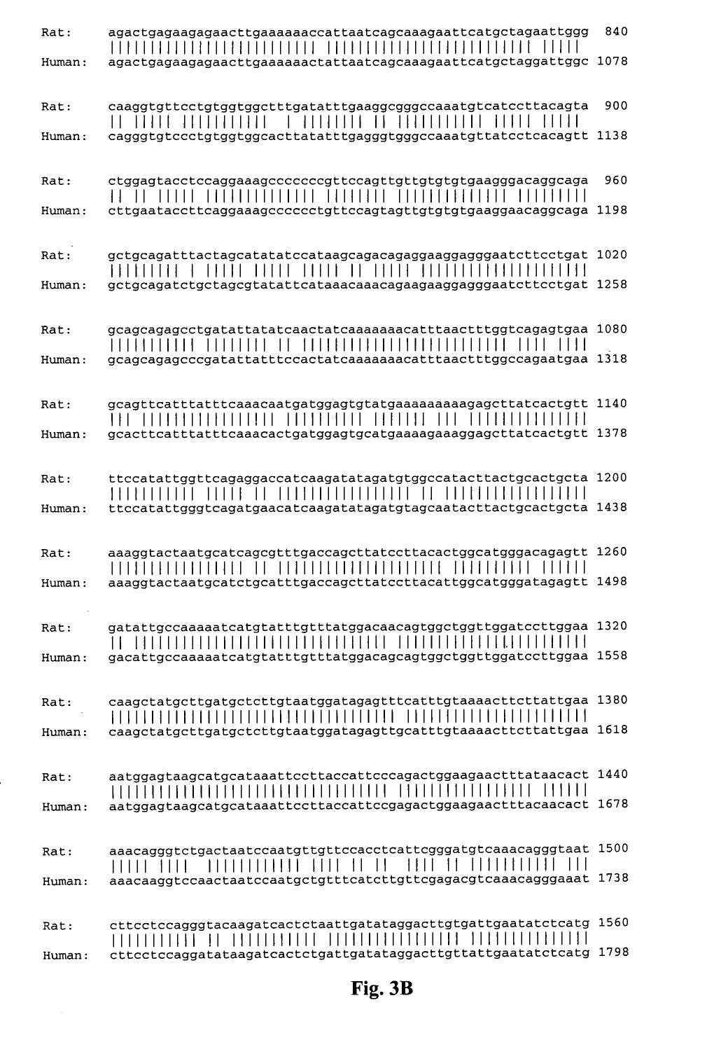

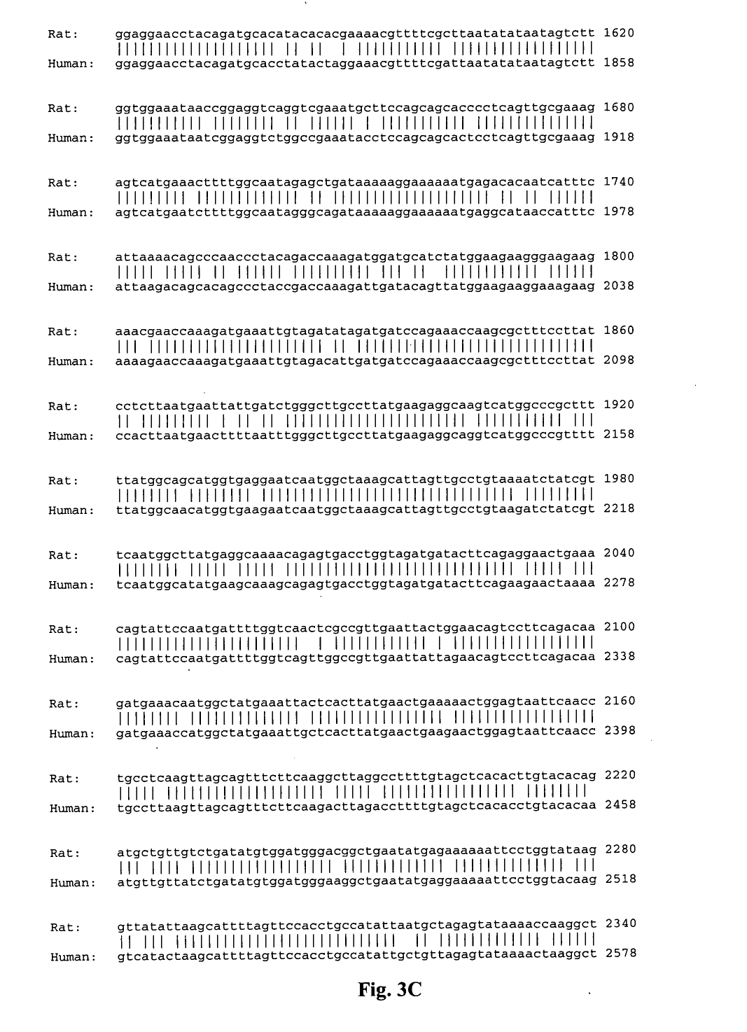

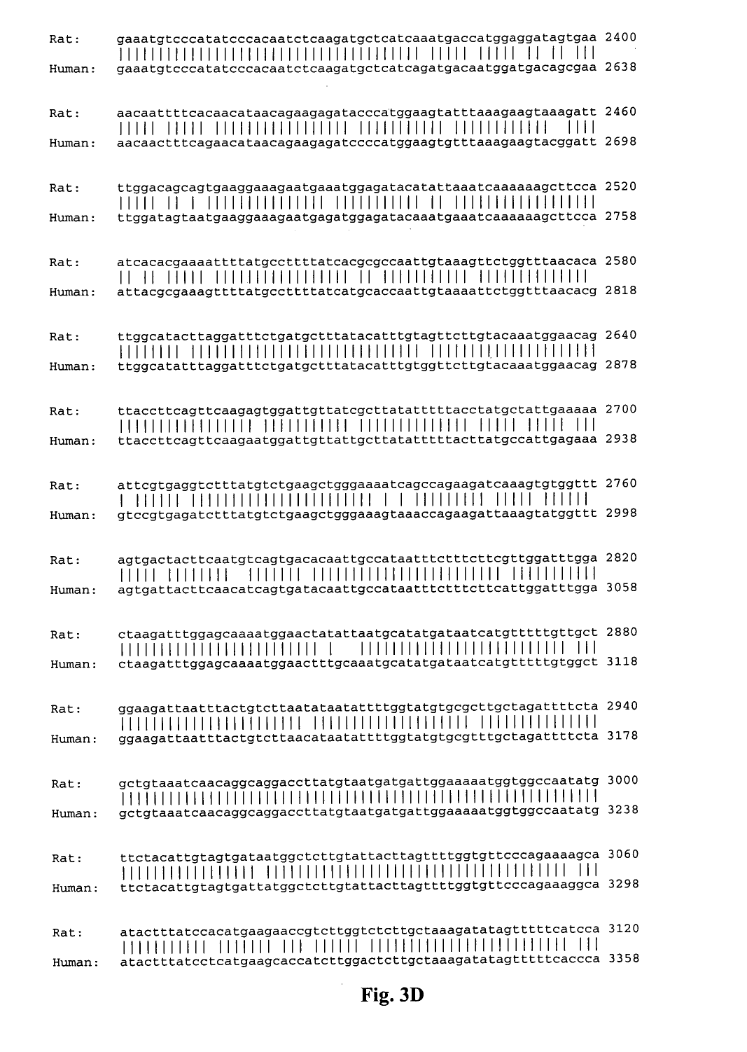

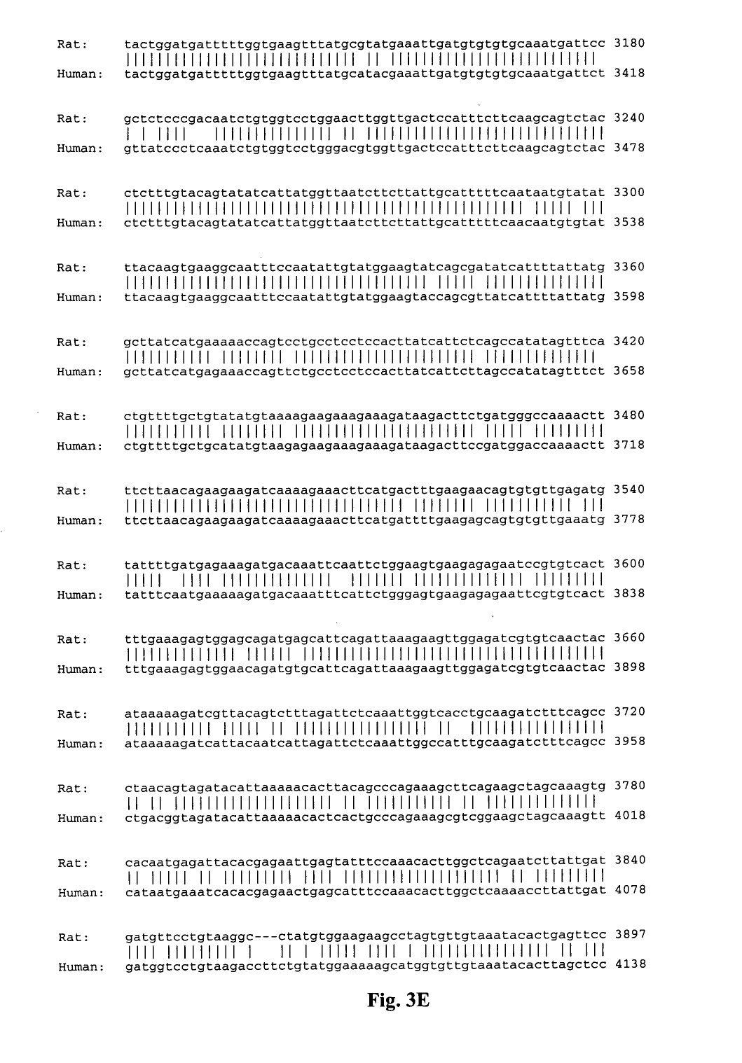

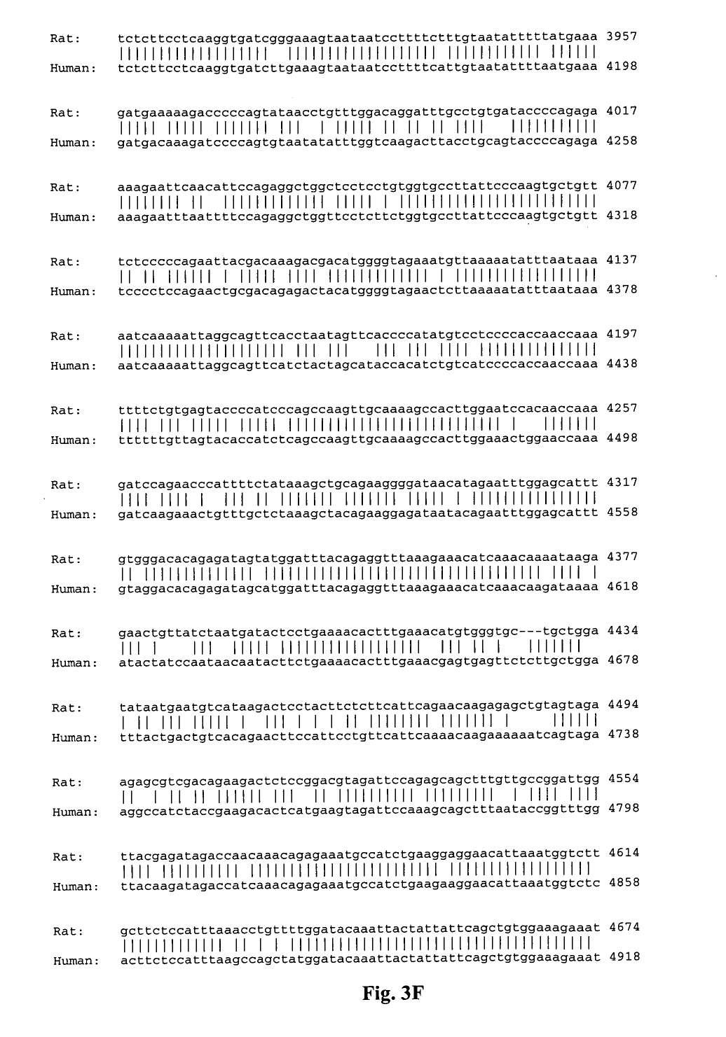

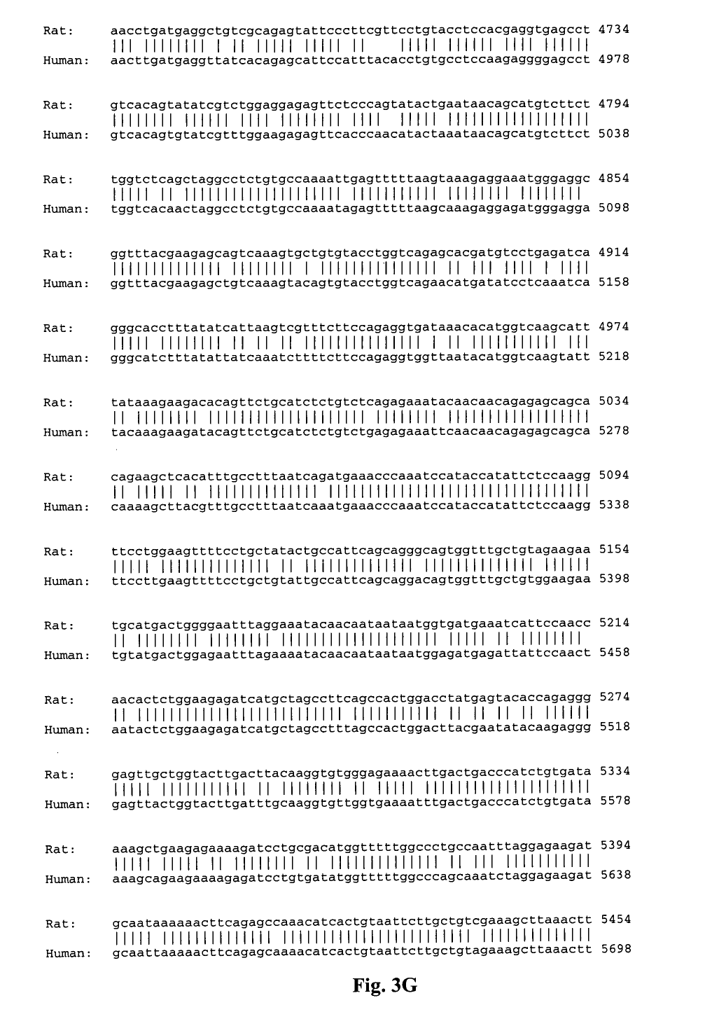

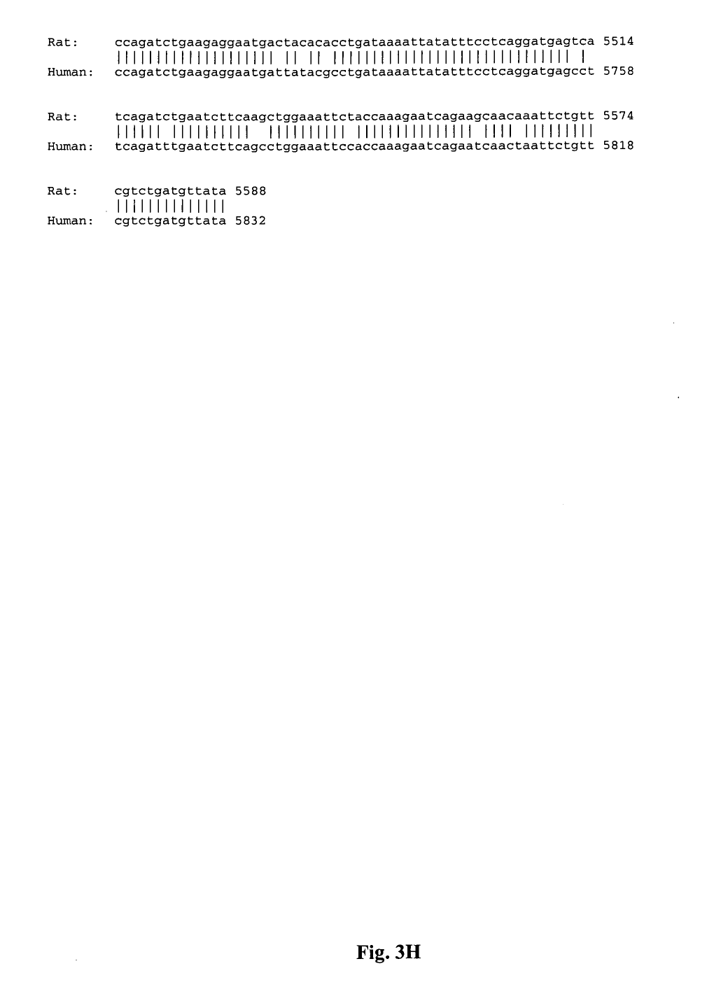

Rat TRPM7 open reading frame (SEQ ID NO:1) was compared with human TRPM7 (NM_{—}017672.2) (SEQ ID NO:15). The results are shown in FIG. 3.

### Example 3

### Protein Sequence Alignment

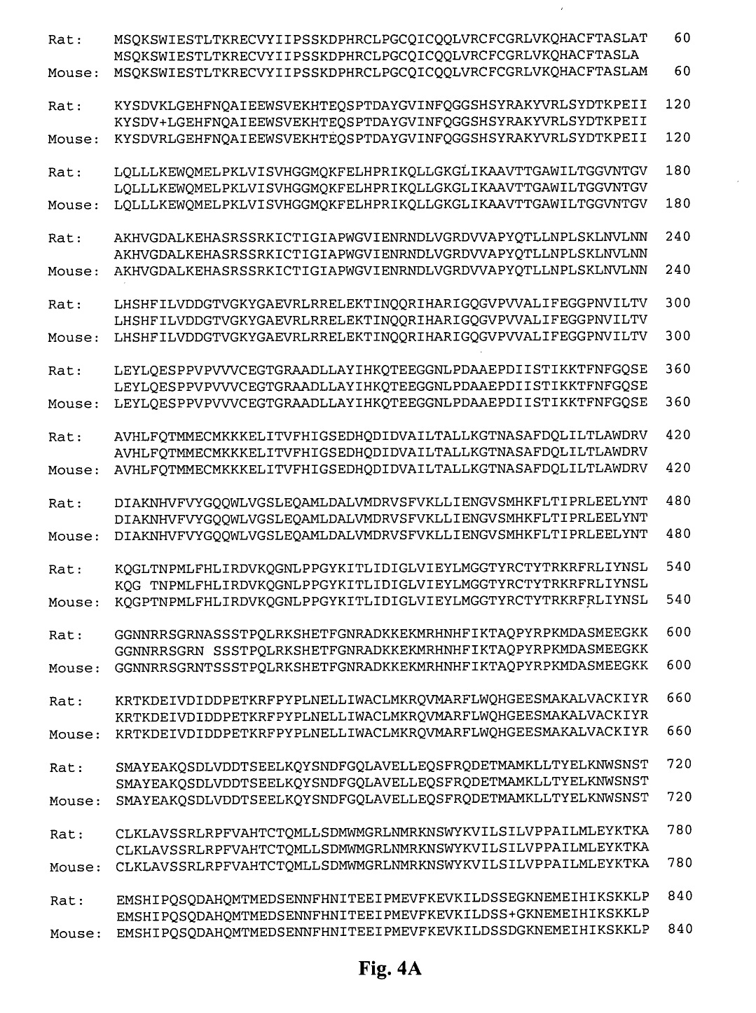

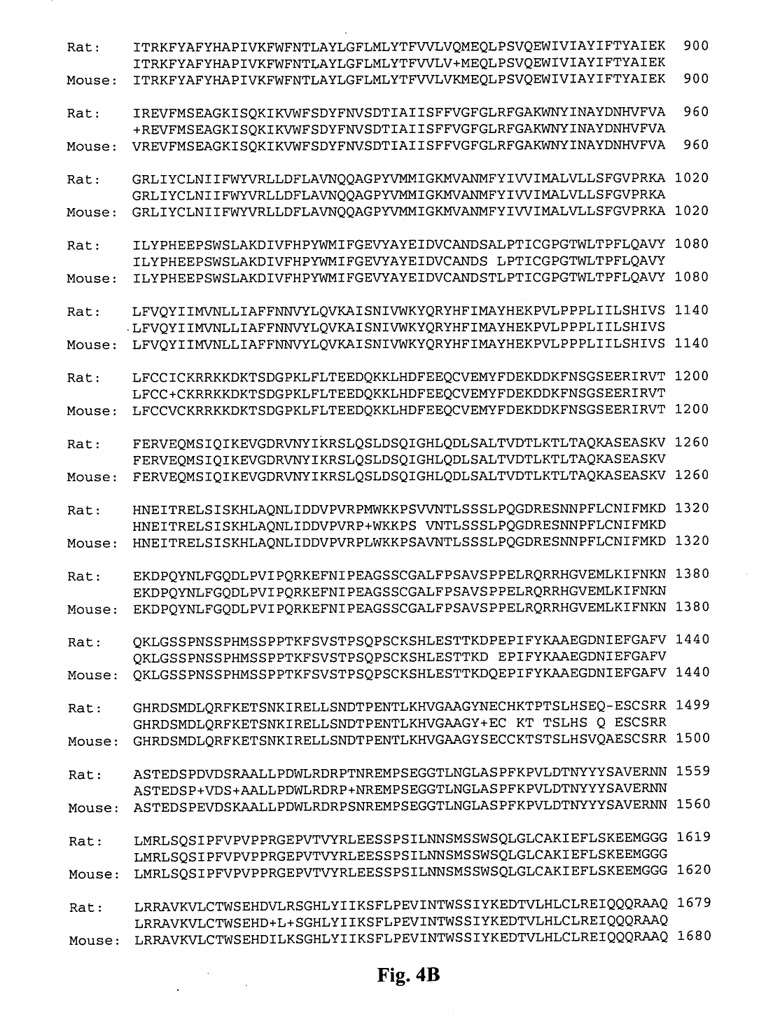

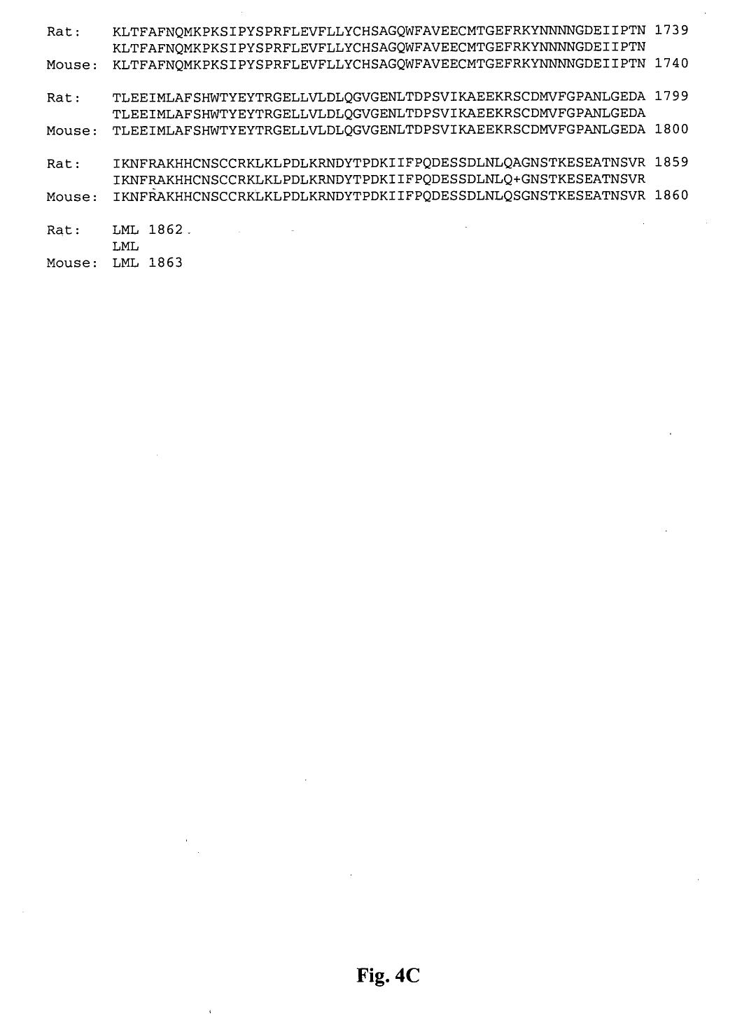

Rat TRPM7 (SEQ ID NO:2) was compared with mouse TRPM7 (SEQ ID NO:16). The results are shown in FIG. 4.

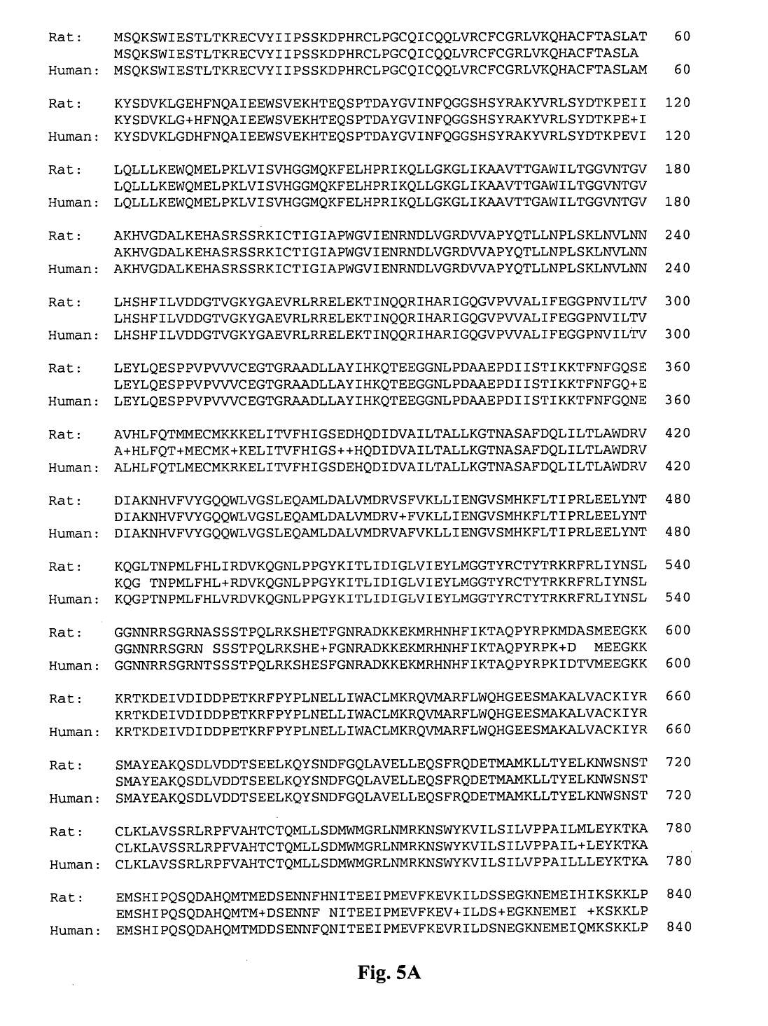

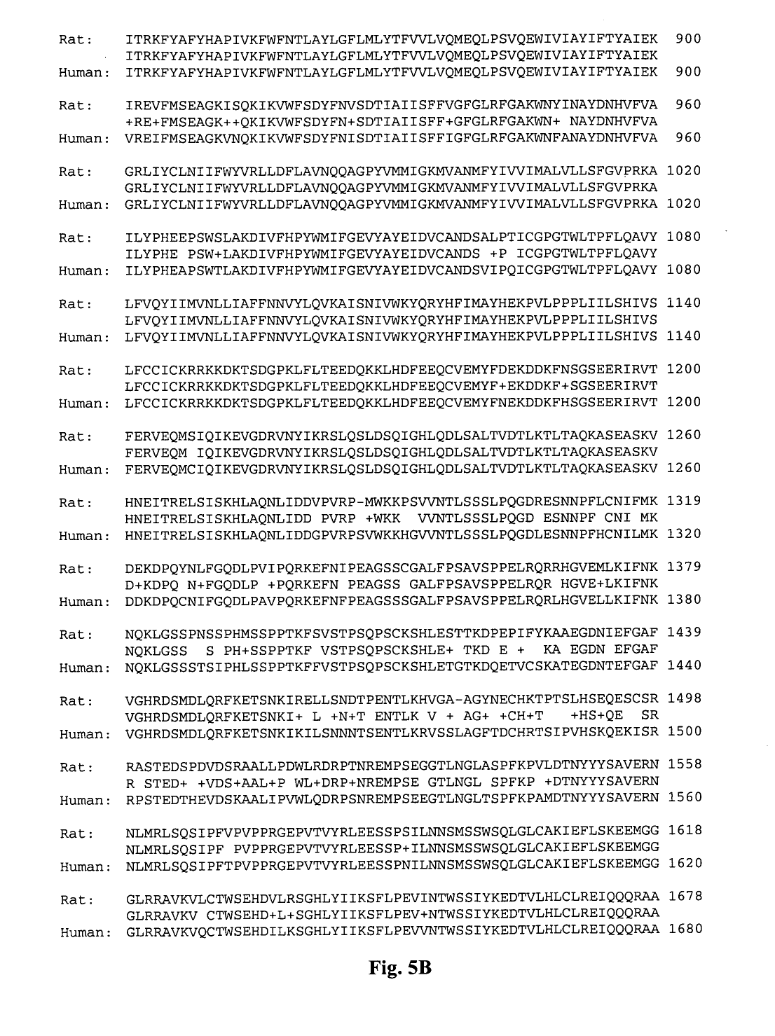

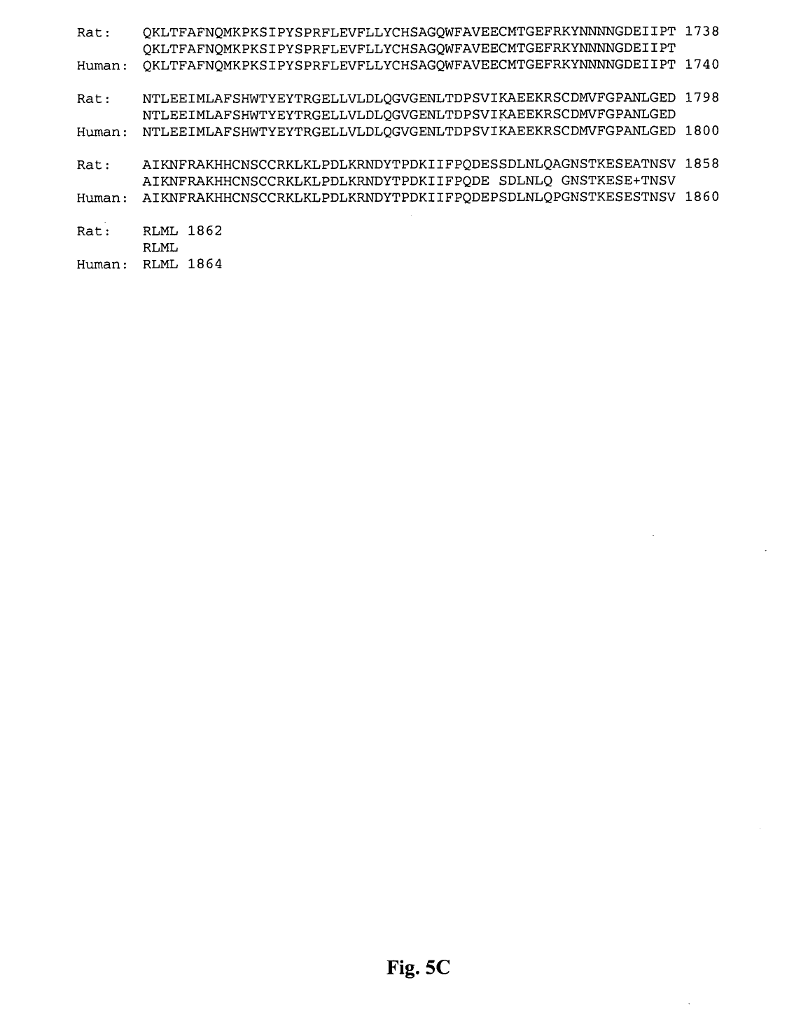

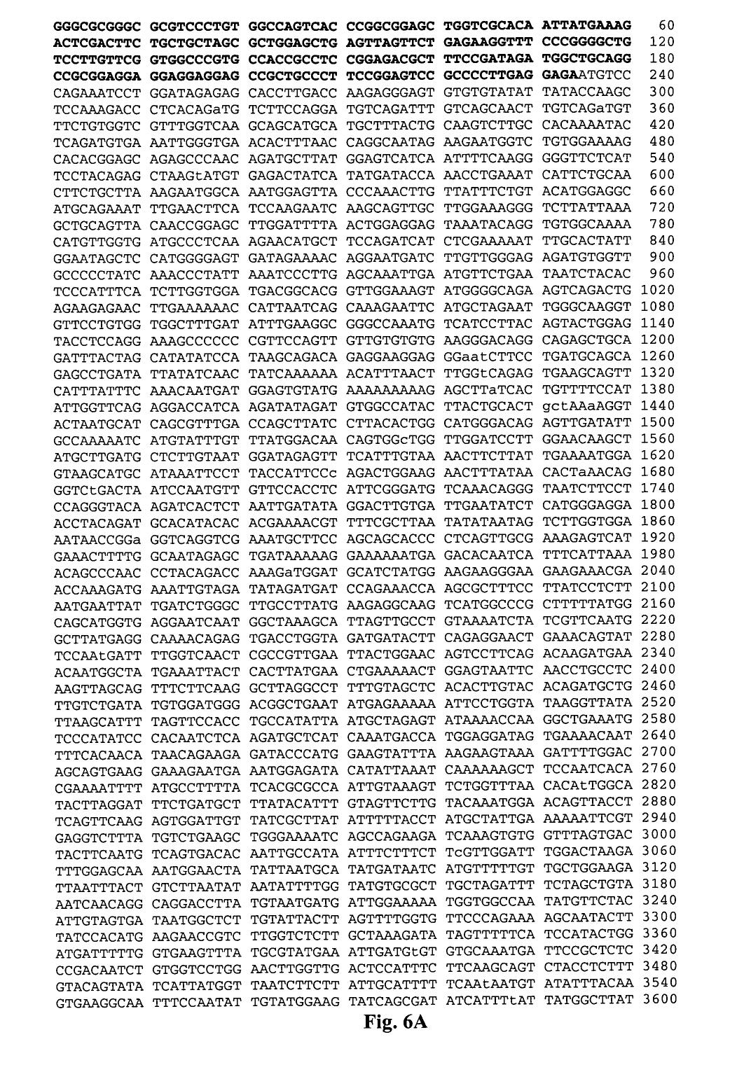

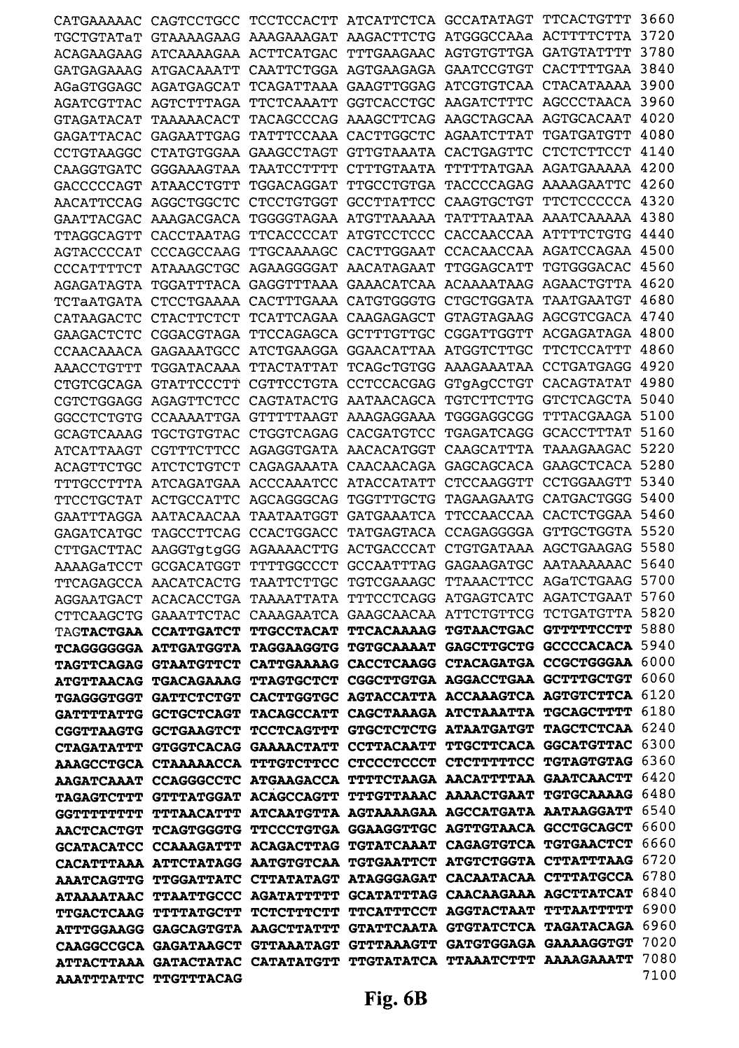

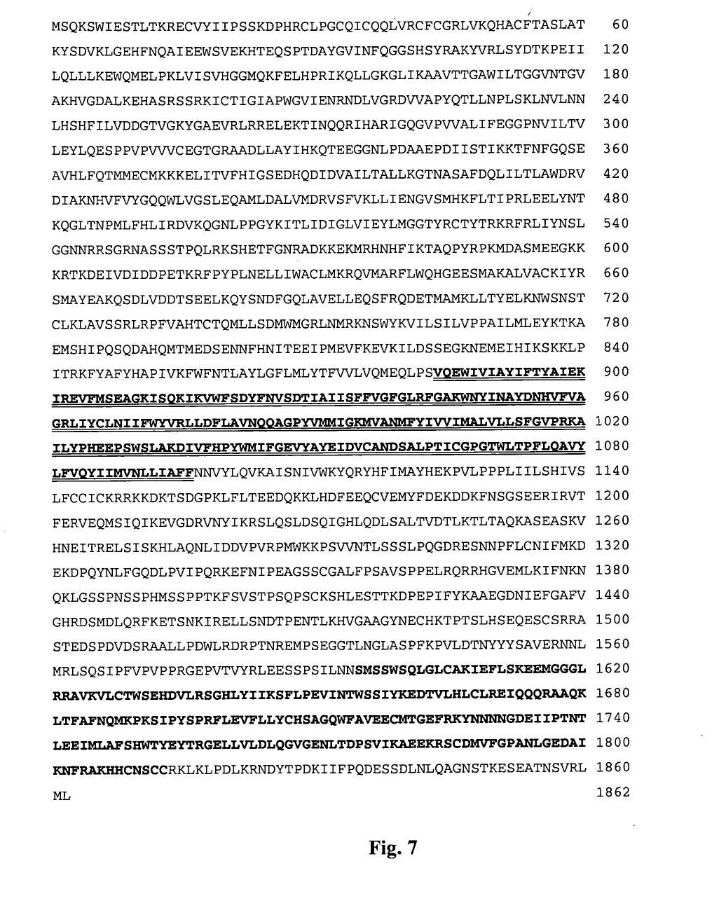

Rat TRPM7 (SEQ ID NO:2) was compared with human TRPM7 (SEQ ID NO:18). The results are shown in FIG. 5.

### Example 4

### Assay for Screening for a Bioactive Agent that Modulates TRPM7 Activity

TRPM7 mediates ischemia induced toxicity. Cells that overexpress TRPM7 should therefore be more sensitive to removal of oxygen and glucose from the medium than control cells. A bioactive agent that inhibits TRPM7 activity will have a protective effect on the cells that have been denied glucose and oxygen. Two exemplary assays that can be used for measuring cell death induced by ischemia include an assay for measuring lactate dehydrogenase (LDH), which is released from dying cells and an assay for measuring ATP in living cells, such as the CellTiter Glo kit® from Promega. Cell death can also be measured by fluorescent measurements of propidium iodide and dihydrorhodamine.

An exemplary LDH assay is as follows. Cells are plated in polylysine coated plate. Plates are used when cells are at least 90% confluent. Media is removed and replaced with 300 ul/well HBSS or DME without O₂ and glucose. 50 μl samples are collected at different time points for LDH evaluation and transferred to a clear 96-well plate. 200 μl of NADH solution (1 mg/10 ml PBS) is added to each well of 50 μl samples. After at least 20 minutes, 10 μl of 22.7 mM pyruvate solution (5 mg/ml PBS) is added and absorbance is read at 340 nm. A candidate bioactive agent can be added before or after deprivation of O₂ or glucose to determine if the agent has any effect on cell survival.

An exemplary CellTiter Glo kit assay is as follows. Cells are plated in polylysine coated plates. Plates are used when cells are at least 90% confluent. Media is removed and replaced with 300 ul/well HBSS or DME without O₂ and glucose. A vial of solution and reagent is thawed. The solution is added to the reagent and mixed well. 100 μl of the reagent mixture is added to each well. The cells are covered and placed on a gyrating platform to mix solution for at least 10 minutes. Luminescence is read in FLEX station, @5 reads=normal resolution. A candidate bioactive agent can be added before deprivation of O₂ and glucose or after to determine if the agent has any effect on cell survival.

Cell death can also be measured by fluorescence measurement of propidium iodide and dihydrorhodamine, respectively using a multiwell plate fluorescence scanner.

### Example 5

### Oxygen-Glucose Deprivation

In order to deprive cells of oxygen and glucose, cells are transferred to an anaerobic chamber containing a 5% CO₂, 10% H₂, and 85% N₂ (<0.2% O₂) atmosphere (Golberg et al., J. Neurosci. 1993 13:3510-3524). Cells are washed with 500 μl of deoxygenated glucose-free bicarbonate solution and maintained anoxic for an appropriate duration at 37° C. Oxygen-glucose deprivation is terminated by washing the cultures with oxygenated glucose-containing (20 mM) bicarbonate solution.

What is claimed:

1. An isolated nucleic acid encoding at least one functional domain of a rat transient receptor potential channel TRPM7, wherein the nucleic acid comprises a polynucleotide sequence having at least 96% identity to SEQ ID NO:1, SEQ ID NO:5, SEQ ID NO:6, or SEQ ID NO:7.

2. The nucleic acid of claim 1 wherein the nucleic acid comprises SEQ ID NO:1.

3. The nucleic acid of claim 1 wherein the nucleic acid comprises SEQ ID NO:5.

4. The nucleic acid of claim 1 wherein the nucleic acid comprises SEQ ID NO:6.

5. The nucleic acid of claim 1 wherein the nucleic acid comprises SEQ ID NO:7.

6. An expression vector comprising the rat nucleic acid of claim 1 operatively linked to a regulatory sequence that controls expression of the polynucleotide in a host cell.

7. The expression vector of claim 6 wherein the polynucleotide is operatively linked to the regulatory sequence in an antisense orientation.

8. The expression vector of claim 6 wherein the polynucleotide is operatively linked to the regulatory sequence in an sense orientation.

9. A host cell comprising the polynucleotide of claim 1 or progeny of the cell.

10. The nucleic acid of claim 1 wherein the nucleic acid is isolated from rat.

11. A method of producing a polypeptide comprising culturing the host cell of claim 9 under conditions such that the polypeptide is expressed, and recovering the polypeptide.

12. An isolated polypeptide encoded by a polynucleotide of claim 1.

13. An isolated rat transient receptor potential channel TRPM7 polypeptide comprising an amino acid sequence having at least 99% identity to SEQ ID NO:2.

14. The polypeptide of claim 10 wherein the polypeptide is SEQ ID NO:2.

15. An ion transport domain comprising an amino acid sequence having at least 99% identity to SEQ ID NO:3.

16. The ion transport domain of claim 15 wherein the amino acid sequence is SEQ ID NO:3.

17. An alpha kinase domain comprising an amino acid sequence having at least 99% identity to SEQ ID NO:4.

18. The alpha kinase domain of claim 17 wherein the amino acid sequence is SEQ ID NO:4.

19. A method of screening bioactive agents comprising:
providing a host cell that expresses a rat TRPM7 gene;
adding a candidate bioactive agent to the cell; and
determining the effect of the candidate bioactive agent on expression of the rat TRPM7 gene.

20. The method of claim 19 wherein the determining comprises comparing the level of expression in the absence of the bioactive agent to the level of expression in the presence of the bioactive agent.

21. A method for screening for a bioactive agent that binds to a rat TRPM7 protein comprising
contacting the rat TRPM7 protein with a candidate bioactive agent; and
determining the binding of the candidate bioactive agent to the rat protein.

22. A method for screening for a bioactive agent that increases or decreases ion flux through a rat TRPM7 channel comprising
contacting a candidate bioactive agent with a TRPM7 protein wherein the TRPM7 protein forms a TRPM7 channel; and
determining the functional effect of the candidate bioactive agent on the TRPM7 channel.

23. The method of claim 22 wherein the determining comprises comparing ion flux in the absence of the bioactive agent to ion flux in the presence of the bioactive agent.

24. The method of claim 22 wherein the ion is Ca²⁺.

25. A method for screening for a bioactive agent that modulates the monovalent or divalent cationic permeability of a rat TRPM7 channel comprising a TRPM7 protein comprising
contacting the rat TRPM7 channel with a candidate bioactive agent;
activating the channel; and
detecting whether the bioactive agent modulates the monovalent or divalent cationic permeability of the channel.

26. The method of claim 25 wherein the monovalent or divalent cationic permeability of the channel is increased by contacting the channel with the bioactive agent.

27. The method of claim 25 wherein the monovalent or divalent cationic permeability of the channel is decreased by contacting the channel with the bioactive agent.

28. A method for screening for a bioactive agent that modulates the monovalent or divalent cationic permeability of a channel comprising a TRPM7 protein comprising
providing a recombinant cell comprising a recombinant nucleic acid encoding a TRPM7 protein and an inducible promoter operably linked thereto;
inducing the recombinant cell to express the TRPM7 protein and form a channel comprising the TRPM7 protein;
contacting the recombinant cell with a candidate bioactive agent;
activating the channel; and
detecting modulation of the monovalent or divalent cation permeability of the channel.

29. The method of claim 28 wherein the monovalent or divalent cationic permeability of the channel is increased by contacting the cell with the bioactive agent.

30. The method of claim 28 wherein the monovalent or divalent cationic permeability of the channel is decreased by contacting the cell with the bioactive agent

31. The method of claim 28 wherein contacting the channel with the bioactive agent alters the membrane potential of a cell comprising the TRPM7 protein.

32. A method for screening for a bioactive agent that modulates the monovalent or divalent cationic permeability of a channel comprising a TRPM7 protein comprising
providing a recombinant cell comprising a recombinant nucleic acid encoding a TRPM7 protein and an inducible promoter operably linked thereto;
inducing the recombinant cell to express the TRPM7 protein and form a channel comprising the TRPM7 protein;
contacting the recombinant cell with a candidate bioactive agent;
activating the channel; and
detecting the monovalent or divalent cation permeability of the channel.

33. The method of claim 32 wherein the detecting comprises comparing the intracellular monovalent or divalent cation levels to intracellular monovalent or divalent cation levels in a cell that does not express the TRPM7 protein but that is in contact with the bioactive agent.

34. A method for screening for a bioactive agent that modulates the activity of a rat TRPM7 protein comprising 4
contacting a candidate bioactive agent with a TRPM7 protein wherein the TRPM7 protein forms a TRPM7 channel;
inducing anoxic neuronal cell death; and
measuring cell death.

35. A method for screening for a bioactive agent that modulates the activity of a rat TRPM7 protein comprising
contacting a candidate bioactive agent with a TRPM7 protein wherein the TRPM7 protein forms a TRPM7 channel;
inducing cell death by denying oxygen and glucose to cells; and
measuring cell death.

36. A method of evaluating the effect of a TRPM7 modulating drug comprising
administering the drug to a mammal;
removing a cell sample from the mammal; and
determining the expression of a rat TRPM7 gene.

37. A method for increasing expression of a rat TRPM7 gene comprising
providing a biological system in which expression of a rat TRPM7 gene is to be increased;
contacting the system with an expression cassette comprising a polynucleotide encoding a rat TRPM7 polypeptide, wherein said polynucleotide is under the control of a promoter operable in eukaryotic cells; and
increasing expression of the gene encoding the protein.

38. A method of screening for a modulator of ischemic injury, comprising
contacting a recombinant cell or cell line that expresses the rat TRPM7 gene with a test compound;
and detecting an increase or a decrease in the amount of cell death.

39. A knock-out rat, wherein an endogenous gene sequence comprising a rat TRPM7 gene is disrupted.

40. An isolated nucleic acid encoding a rat transient receptor potential channel TRPM7, wherein the nucleic acid comprises SEQ ID NO:1 or a conservatively modified variant thereof.


SEQUENCE LISTING (19 sequences)

SEQ ID NO: 1 - DNA, 5589 bp, Rattus
atgtcccagaaatcctggatagagagcaccttgaccaagagggagtgtgtgtatattataccaagctccaaagaccctcacagatgtcttccaggatgtcagatttgtcagcaacttgtcagatgtttctgtggtcgtttggtcaagcagcatgcatgctttactgcaagtcttgccacaaaatactcagatgtgaaattgggtgaacactttaaccaggcaatagaagaatggtctgtggaaaagcacacggagcagagcccaacagatgcttatggagtcatcaattttcaagggggttctcattcctacagagctaagtatgtgagactatcatatgataccaaacctgaaatcattctgcaacttctgcttaaagaatggcaaatggagttacccaaacttgttatttctgtacatggaggcatgcagaaatttgaacttcatccaagaatcaagcagttgcttggaaagggtcttattaaagctgcagttacaaccggagcttggattttaactggaggagtaaatacaggtgtggcaaaacatgttggtgatgccctcaaagaacatgcttccagatcatctcgaaaaatttgcactattggaatagctccatggggagtgatagaaaacaggaatgatcttgttgggagagatgtggttgccccctatcaaaccctattaaatcccttgagcaaattgaatgttctgaataatctacactcccatttcatcttggtggatgacggcacggttggaaagtatggggcagaagtcagactgagaagagaacttgaaaaaaccattaatcagcaaagaattcatgctagaattgggcaaggtgttcctgtggtggctttgatatttgaaggcgggccaaatgtcatccttacagtactggagtacctccaggaaagcccccccgttccagttgttgtgtgtgaagggacaggcagagctgcagatttactagcatatatccataagcagacagaggaaggagggaatcttcctgatgcagcagagcctgatattatatcaactatcaaaaaaacatttaactttggtcagagtgaagcagttcatttatttcaaacaatgatggagtgtatgaaaaaaaaagagcttatcactgttttccatattggttcagaggaccatcaagatatagatgtggccatacttactgcactgctaaaaggtactaatgcatcagcgtttgaccagcttatccttacactggcatgggacagagttgatattgccaaaaatcatgtatttgtttatggacaacagtggctggttggatccttggaacaagctatgcttgatgctcttgtaatggatagagtttcatttgtaaaacttcttattgaaaatggagtaagcatgcataaattccttaccattcccagactggaagaactttataacactaaacagggtctgactaatccaatgttgttccacctcattcgggatgtcaaacagggtaatcttcctccagggtacaagatcactctaattgatataggacttgtgattgaatatctcatgggaggaacctacagatgcacatacacacgaaaacgttttcgcttaatatataatagtcttggtggaaataaccggaggtcaggtcgaaatgcttccagcagcacccctcagttgcgaaagagtcatgaaacttttggcaatagagctgataaaaaggaaaaaatgagacacaatcatttcattaaaacagcccaaccctacagaccaaagatggatgcatctatggaagaagggaagaagaaacgaaccaaagatgaaattgtagatatagatgatccagaaaccaagcgctttccttatcctcttaatgaattattgatctgggcttgccttatgaagaggcaagtcatggcccgctttttatggcagcatggtgaggaatcaatggctaaagcattagttgcctgtaaaatctatcgttcaatggcttatgaggcaaaacagagtgacctggtagatgatacttcagaggaactgaaacagtattccaatgattttggtcaactcgccgttgaattactggaacagtccttcagacaagatgaaacaatggctatgaaattactcacttatgaactgaaaaactggagtaattcaacctgcctcaagttagcagtttcttcaaggcttaggccttttgtagctcacacttgtacacagatgctgttgtctgatatgtggatgggacggctgaatatgagaaaaaattcctggtataaggttatattaagcattttagttccacctgccatattaatgctagagtataaaaccaaggctgaaatgtcccatatcccacaatctcaagatgctcatcaaatgaccatggaggatagtgaaaacaattttcacaacataacagaagagatacccatggaagtatttaaagaagtaaagattttggacagcagtgaaggaaagaatgaaatggagatacatattaaatcaaaaaagcttccaatcacacgaaaattttatgccttttatcacgcgccaattgtaaagttctggtttaacacattggcatacttaggatttctgatgctttatacatttgtagttcttgtacaaatggaacagttaccttcagttcaagagtggattgttatcgcttatatttttacctatgctattgaaaaaattcgtgaggtctttatgtctgaagctgggaaaatcagccagaagatcaaagtgtggtttagtgactacttcaatgtcagtgacacaattgccataatttctttcttcgttggatttggactaagatttggagcaaaatggaactatattaatgcatatgataatcatgtttttgttgctggaagattaatttactgtcttaatataatattttggtatgtgcgcttgctagattttctagctgtaaatcaacaggcaggaccttatgtaatgatgattggaaaaatggtggccaatatgttctacattgtagtgataatggctcttgtattacttagttttggtgttcccagaaaagcaatactttatccacatgaagaaccgtcttggtctcttgctaaagatatagtttttcatccatactggatgatttttggtgaagtttatgcgtatgaaattgatgtgtgtgcaaatgattccgctctcccgacaatctgtggtcctggaacttggttgactccatttcttcaagcagtctacctctttgtacagtatatcattatggttaatcttcttattgcatttttcaataatgtatatttacaagtgaaggcaatttccaatattgtatggaagtatcagcgatatcattttattatggcttatcatgaaaaaccagtcctgcctcctccacttatcattctcagccatatagtttcactgttttgctgtatatgtaaaagaagaaagaaagataagacttctgatgggccaaaacttttcttaacagaagaagatcaaaagaaacttcatgactttgaagaacagtgtgttgagatgtattttgatgagaaagatgacaaattcaattctggaagtgaagagagaatccgtgtcacttttgaaagagtggagcagatgagcattcagattaaagaagttggagatcgtgtcaactacataaaaagatcgttacagtctttagattctcaaattggtcacctgcaagatctttcagccctaacagtagatacattaaaaacacttacagcccagaaagcttcagaagctagcaaagtgcacaatgagattacacgagaattgagtatttccaaacacttggctcagaatcttattgatgatgttcctgtaaggcctatgtggaagaagcctagtgttgtaaatacactgagttcctctcttcctcaaggtgatcgggaaagtaataatccttttctttgtaatatttttatgaaagatgaaaaagacccccagtataacctgtttggacaggatttgcctgtgataccccagagaaaagaattcaacattccagaggctggctcctcctgtggtgccttattcccaagtgctgtttctcccccagaattacgacaaagacgacatggggtagaaatgttaaaaatatttaataaaaatcaaaaattaggcagttcacctaatagttcaccccatatgtcctccccaccaaccaaattttctgtgagtaccccatcccagccaagttgcaaaagccacttggaatccacaaccaaagatccagaacccattttctataaagctgcagaaggggataacatagaatttggagcatttgtgggacacagagatagtatggatttacagaggtttaaagaaacatcaaacaaaataagagaactgttatctaatgatactcctgaaaacactttgaaacatgtgggtgctgctggatataatgaatgtcataagactcctacttctcttcattcagaacaagagagctgtagtagaagagcgtcgacagaagactctccggacgtagattccagagcagctttgttgccggattggttacgagatagaccaacaaacagagaaatgccatctgaaggaggaacattaaatggtcttgcttctccatttaaacctgttttggatacaaattactattattcagctgtggaaagaaataacctgatgaggctgtcgcagagtattcccttcgttcctgtacctccacgaggtgagcctgtcacagtatatcgtctggaggagagttctcccagtatactgaataacagcatgtcttcttggtctcagctaggcctctgtgccaaaattgagtttttaagtaaagaggaaatgggaggcggtttacgaagagcagtcaaagtgctgtgtacctggtcagagcacgatgtcctgagatcagggcacctttatatcattaagtcgtttcttccagaggtgataaacacatggtcaagcatttataaagaagacacagttctgcatctctgtctcagagaaatacaacaacagagagcagcacagaagctcacatttgcctttaatcagatgaaacccaaatccataccatattctccaaggttcctggaagttttcctgctatactgccattcagcagggcagtggtttgctgtagaagaatgcatgactggggaatttaggaaatacaacaataataatggtgatgaaatcattccaaccaacactctggaagagatcatgctagccttcagccactggacctatgagtacaccagaggggagttgctggtacttgacttacaaggtgtgggagaaaacttgactgacccatctgtgataaaagctgaagagaaaagatcctgcgacatggtttttggccctgccaatttaggagaagatgcaataaaaaacttcagagccaaacatcactgtaattcttgctgtcgaaagcttaaacttccagatctgaagaggaatgactacacacctgataaaattatatttcctcaggatgagtcatcagatctgaatcttcaagctggaaattctaccaaagaatcagaagcaacaaattctgttcgtctgatgttatag

SEQ ID NO: 2 - protein, 1862 aa, Rattus
MSQKSWIESTLTKRECVYIIPSSKDPHRCLPGCQICQQLVRCFCGRLVKQHACFTASLATKYSDVKLGEHFNQAIEEWSVEKHTEQSPTDAYGVINFQGGSHSYRAKYVRLSYDTKPEIILQLLLKEWQMELPKLVISVHGGMQKFELHPRIKQLLGKGLIKAAVTTGAWILTGGVNTGVAKHVGDALKEHASRSSRKICTIGIAPWGVIENRNDLVGRDVVAPYQTLLNPLSKLNVLNNLHSHFILVDDGTVGKYGAEVRLRRELEKTINQQRIHARIGQGVPVVALIFEGGPNVILTVLEYLQESPPVPVVVCEGTGRAADLLAYIHKQTEEGGNLPDAAEPDIISTIKKTFNFGQSEAVHLFQTMMECMKKKELITVFHIGSEDHQDIDVAILTALLKGTNASAFDQLILTLAWDRVDIAKNHVFVYGQQWLVGSLEQAMLDALVMDRVSFVKLLIENGVSMHKFLTIPRLEELYNTKQGLTNPMLFHLIRDVKQGNLPPGYKITLIDIGLVIEYLMGGTYRCTYTRKRFRLIYNSLGGNNRRSGRNASSSTPQLRKSHETFGNRADKKEKMRHNHFIKTAQPYRPKMDASMEEGKKKRTKDEIVDIDDPETKRFPYPLNELLIWACLMKRQVMARFLWQHGEESMAKALVACKIYRSMAYEAKQSDLVDDTSEELKQYSNDFGQLAVELLEQSFRQDETMAMKLLTYELKNWSNSTCLKLAVSSRLRPFVAHTCTQMLLSDMWMGRLNMRKNSWYKVILSILVPPAILMLEYKTKAEMSHIPQSQDAHQMTMEDSENNFHNITEEIPMEVFKEVKILDSSEGKNEMEIHIKSKKLPITRKFYAFYHAPIVKFWFNTLAYLGFLMLYTFVVLVQMEQLPSVQEWIVIAYIFTYAIEKIREVFMSEAGKISQKIKVWFSDYFNVSDTIAIISFFVGFGLRFGAKWNYINAYDNHVFVAGRLIYCLNIIFWYVRLLDFLAVNQQAGPYVMMIGKMVANMFYIVVIMALVLLSFGVPRKAILYPHEEPSWSLAKDIVFHPYWMIFGEVYAYEIDVCANDSALPTICGPGTWLTPFLQAVYLFVQYIIMVNLLIAFFNNVYLQVKAISNIVWKYQRYHFIMAYHEKPVLPPPLIILSHIVSLFCCICKRRKKDKTSDGPKLFLTEEDQKKLHDFEEQCVEMYFDEKDDKFNSGSEERIRVTFERVEQMSIQIKEVGDRVNYIKRSLQSLDSQIGHLQDLSALTVDTLKTLTAQKASEASKVHNEITRELSISKHLAQNLIDDVPVRPMWKKPSVVNTLSSSLPQGDRESNNPFLCNIFMKDEKDPQYNLFGQDLPVIPQRKEFNIPEAGSSCGALFPSAVSPPELRQRRHGVEMLKIFNKNQKLGSSPNSSPHMSSPPTKFSVSTPSQPSCKSHLESTTKDPEPIFYKAAEGDNIEFGAFVGHRDSMDLQRFKETSNKIRELLSNDTPENTLKHVGAAGYNECHKTPTSLHSEQESCSRRASTEDSPDVDSRAALLPDWLRDRPTNREMPSEGGTLNGLASPFKPVLDTNYYYSAVERNNLMRLSQSIPFVPVPPRGEPVTVYRLEESSPSILNNSMSSWSQLGLCAKIEFLSKEEMGGGLRRAVKVLCTWSEHDVLRSGHLYIIKSFLPEVINTWSSIYKEDTVLHLCLREIQQQRAAQKLTFAFNQMKPKSIPYSPRFLEVFLLYCHSAGQWFAVEECMTGEFRKYNNNNGDEIIPTNTLEEIMLAFSHWTYEYTRGELLVLDLQGVGENLTDPSVIKAEEKRSCDMVFGPANLGEDAIKNFRAKHHCNSCCRKLKLPDLKRNDYTPDKIIFPQDESSDLNLQAGNSTKESEATNSVRLML

SEQ ID NO: 3 - protein, 213 aa, Rattus
VQEWIVIAYIFTYAIEKIREVFMSEAGKISQKIKVWFSDYFNVSDTIAIISFFVGFGLRFGAKWNYINAYDNHVFVAGRLIYCLNIIFWYVRLLDFLAVNQQAGPYVMMIGKMVANMFYIVVIMALVLLSFGVPRKAILYPHEEPSWSLAKDIVFHPYWMIFGEVYAYEIDVCANDSALPTICGPGTWLTPFLQAVYLFVQYIIMVNLLIAFF

SEQ ID NO: 4 - protein, 219 aa, Rattus
SMSSWSQLGLCAKIEFLSKEEMGGGLRRAVKVLCTWSEHDVLRSGHLYIIKSFLPEVINTWSSIYKEDTVLHLCLREIQQQRAAQKLTFAFNQMKPKSIPYSPRFLEVFLLYCHSAGQWFAVEECMTGEFRKYNNNNGDEIIPTNTLEEIMLAFSHWTYEYTRGELLVLDLQGVGENLTDPSVIKAEEKRSCDMVFGPANLGEDAIKNFRAKHHCNSCC

SEQ ID NO: 5 - DNA, 7100 bp, Rattus
gggcgcgggcgcgtccctgtggccagtcacccggcggagctggtcgcacaattatgaaagactcgacttctgctgctagcgctggagctgagttagttctgagaaggtttcccggggctgtccttgttcggtggcccgtgccaccgcctccggagacgctttccgatagatggctgcaggccgcggaggaggaggaggagccgctgcccttccggagtccgccccttgaggagaatgtcccagaaatcctggatagagagcaccttgaccaagagggagtgtgtgtatattataccaagctccaaagaccctcacagatgtcttccaggatgtcagatttgtcagcaacttgtcagatgtttctgtggtcgtttggtcaagcagcatgcatgctttactgcaagtcttgccacaaaatactcagatgtgaaattgggtgaacactttaaccaggcaatagaagaatggtctgtggaaaagcacacggagcagagcccaacagatgcttatggagtcatcaattttcaagggggttctcattcctacagagctaagtatgtgagactatcatatgataccaaacctgaaatcattctgcaacttctgcttaaagaatggcaaatggagttacccaaacttgttatttctgtacatggaggcatgcagaaatttgaacttcatccaagaatcaagcagttgcttggaaagggtcttattaaagctgcagttacaaccggagcttggattttaactggaggagtaaatacaggtgtggcaaaacatgttggtgatgccctcaaagaacatgcttccagatcatctcgaaaaatttgcactattggaatagctccatggggagtgatagaaaacaggaatgatcttgttgggagagatgtggttgccccctatcaaaccctattaaatcccttgagcaaattgaatgttctgaataatctacactcccatttcatcttggtggatgacggcacggttggaaagtatggggcagaagtcagactgagaagagaacttgaaaaaaccattaatcagcaaagaattcatgctagaattgggcaaggtgttcctgtggtggctttgatatttgaaggcgggccaaatgtcatccttacagtactggagtacctccaggaaagcccccccgttccagttgttgtgtgtgaagggacaggcagagctgcagatttactagcatatatccataagcagacagaggaaggagggaatcttcctgatgcagcagagcctgatattatatcaactatcaaaaaaacatttaactttggtcagagtgaagcagttcatttatttcaaacaatgatggagtgtatgaaaaaaaaagagcttatcactgttttccatattggttcagaggaccatcaagatatagatgtggccatacttactgcactgctaaaaggtactaatgcatcagcgtttgaccagcttatccttacactggcatgggacagagttgatattgccaaaaatcatgtatttgtttatggacaacagtggctggttggatccttggaacaagctatgcttgatgctcttgtaatggatagagtttcatttgtaaaacttcttattgaaaatggagtaagcatgcataaattccttaccattcccagactggaagaactttataacactaaacagggtctgactaatccaatgttgttccacctcattcgggatgtcaaacagggtaatcttcctccagggtacaagatcactctaattgatataggacttgtgattgaatatctcatgggaggaacctacagatgcacatacacacgaaaacgttttcgcttaatatataatagtcttggtggaaataaccggaggtcaggtcgaaatgcttccagcagcacccctcagttgcgaaagagtcatgaaacttttggcaatagagctgataaaaaggaaaaaatgagacacaatcatttcattaaaacagcccaaccctacagaccaaagatggatgcatctatggaagaagggaagaagaaacgaaccaaagatgaaattgtagatatagatgatccagaaaccaagcgctttccttatcctcttaatgaattattgatctgggcttgccttatgaagaggcaagtcatggcccgctttttatggcagcatggtgaggaatcaatggctaaagcattagttgcctgtaaaatctatcgttcaatggcttatgaggcaaaacagagtgacctggtagatgatacttcagaggaactgaaacagtattccaatgattttggtcaactcgccgttgaattactggaacagtccttcagacaagatgaaacaatggctatgaaattactcacttatgaactgaaaaactggagtaattcaacctgcctcaagttagcagtttcttcaaggcttaggccttttgtagctcacacttgtacacagatgctgttgtctgatatgtggatgggacggctgaatatgagaaaaaattcctggtataaggttatattaagcattttagttccacctgccatattaatgctagagtataaaaccaaggctgaaatgtcccatatcccacaatctcaagatgctcatcaaatgaccatggaggatagtgaaaacaattttcacaacataacagaagagatacccatggaagtatttaaagaagtaaagattttggacagcagtgaaggaaagaatgaaatggagatacatattaaatcaaaaaagcttccaatcacacgaaaattttatgccttttatcacgcgccaattgtaaagttctggtttaacacattggcatacttaggatttctgatgctttatacatttgtagttcttgtacaaatggaacagttaccttcagttcaagagtggattgttatcgcttatatttttacctatgctattgaaaaaattcgtgaggtctttatgtctgaagctgggaaaatcagccagaagatcaaagtgtggtttagtgactacttcaatgtcagtgacacaattgccataatttctttcttcgttggatttggactaagatttggagcaaaatggaactatattaatgcatatgataatcatgtttttgttgctggaagattaatttactgtcttaatataatattttggtatgtgcgcttgctagattttctagctgtaaatcaacaggcaggaccttatgtaatgatgattggaaaaatggtggccaatatgttctacattgtagtgataatggctcttgtattacttagttttggtgttcccagaaaagcaatactttatccacatgaagaaccgtcttggtctcttgctaaagatatagtttttcatccatactggatgatttttggtgaagtttatgcgtatgaaattgatgtgtgtgcaaatgattccgctctcccgacaatctgtggtcctggaacttggttgactccatttcttcaagcagtctacctctttgtacagtatatcattatggttaatcttcttattgcatttttcaataatgtatatttacaagtgaaggcaatttccaatattgtatggaagtatcagcgatatcattttattatggcttatcatgaaaaaccagtcctgcctcctccacttatcattctcagccatatagtttcactgttttgctgtatatgtaaaagaagaaagaaagataagacttctgatgggccaaaacttttcttaacagaagaagatcaaaagaaacttcatgactttgaagaacagtgtgttgagatgtattttgatgagaaagatgacaaattcaattctggaagtgaagagagaatccgtgtcacttttgaaagagtggagcagatgagcattcagattaaagaagttggagatcgtgtcaactacataaaaagatcgttacagtctttagattctcaaattggtcacctgcaagatctttcagccctaacagtagatacattaaaaacacttacagcccagaaagcttcagaagctagcaaagtgcacaatgagattacacgagaattgagtatttccaaacacttggctcagaatcttattgatgatgttcctgtaaggcctatgtggaagaagcctagtgttgtaaatacactgagttcctctcttcctcaaggtgatcgggaaagtaataatccttttctttgtaatatttttatgaaagatgaaaaagacccccagtataacctgtttggacaggatttgcctgtgataccccagagaaaagaattcaacattccagaggctggctcctcctgtggtgccttattcccaagtgctgtttctcccccagaattacgacaaagacgacatggggtagaaatgttaaaaatatttaataaaaatcaaaaattaggcagttcacctaatagttcaccccatatgtcctccccaccaaccaaattttctgtgagtaccccatcccagccaagttgcaaaagccacttggaatccacaaccaaagatccagaacccattttctataaagctgcagaaggggataacatagaatttggagcatttgtgggacacagagatagtatggatttacagaggtttaaagaaacatcaaacaaaataagagaactgttatctaatgatactcctgaaaacactttgaaacatgtgggtgctgctggatataatgaatgtcataagactcctacttctcttcattcagaacaagagagctgtagtagaagagcgtcgacagaagactctccggacgtagattccagagcagctttgttgccggattggttacgagatagaccaacaaacagagaaatgccatctgaaggaggaacattaaatggtcttgcttctccatttaaacctgttttggatacaaattactattattcagctgtggaaagaaataacctgatgaggctgtcgcagagtattcccttcgttcctgtacctccacgaggtgagcctgtcacagtatatcgtctggaggagagttctcccagtatactgaataacagcatgtcttcttggtctcagctaggcctctgtgccaaaattgagtttttaagtaaagaggaaatgggaggcggtttacgaagagcagtcaaagtgctgtgtacctggtcagagcacgatgtcctgagatcagggcacctttatatcattaagtcgtttcttccagaggtgataaacacatggtcaagcatttataaagaagacacagttctgcatctctgtctcagagaaatacaacaacagagagcagcacagaagctcacatttgcctttaatcagatgaaacccaaatccataccatattctccaaggttcctggaagttttcctgctatactgccattcagcagggcagtggtttgctgtagaagaatgcatgactggggaatttaggaaatacaacaataataatggtgatgaaatcattccaaccaacactctggaagagatcatgctagccttcagccactggacctatgagtacaccagaggggagttgctggtacttgacttacaaggtgtgggagaaaacttgactgacccatctgtgataaaagctgaagagaaaagatcctgcgacatggtttttggccctgccaatttaggagaagatgcaataaaaaacttcagagccaaacatcactgtaattcttgctgtcgaaagcttaaacttccagatctgaagaggaatgactacacacctgataaaattatatttcctcaggatgagtcatcagatctgaatcttcaagctggaaattctaccaaagaatcagaagcaacaaattctgttcgtctgatgttatagtactgaaccattgatctttgcctacatttcacaaaagtgtaactgacgtttttcctttcaggggggaattgatggtataggaaggtgtgtgcaaaatgagcttgctggccccacacatagttcagaggtaatgttctcattgaaaagcacctcaaggctacagatgaccgctgggaaatgttaacagtgacagaaagttagtgctctcggcttgtgaaggacctgaagctttgctgttgagggtggtgattctctgtcacttggtgcagtaccattaaccaaagtcaagtgtcttcagattttattggctgctcagttacagccattcagctaaagaatctaaattatgcagcttttcggttaagtggctgaagtcttcctcagtttgtgctctctgataatgatgttagctctcaactagatatttgtggtcacaggaaaactattccttacaattttgcttcacaggcatgttacaaagcctgcactaaaaaccatttgtcttccctccctccctctctttttcctgtagtgtagaagatcaaatccagggcctcatgaagaccattttctaagaaacattttaagaatcaactttagagtctttgtttatggatacagccagtttttgttaaacaaaactgaattgtgcaaaagggtttttttttttaacatttatcaatgttaagtaaaagaaagccatgataaataaggattaactcactgttcagtgggtgttccctgtgaggaaggttgcagttgtaacagcctgcagctgcatacatccccaaagatttacagacttagtgtatcaaatcagagtgtcatgtgaactctcacatttaaaattctataggaatgtgtcaatgtgaattctatgtctggtacttatttaagaaatcagttgttggattatccttatatagtatagggagatcacaatacaactttatgccaataaaataacttaattgcccagatatttttgcatatttagcaacaagaaaagcttatcatttgactcaagttttatgctttctctttcttttcatttcctaggtactaattttaatttttatttggaagggagcagtgtaaagcttatttgtattcaatagtgtatctcatagatacagacaaggccgcagagataagctgttaaatagtgtttaaagttgatgtggagagaaaaggtgtattacttaaagatactataccatatatgttttgtatatcattaaatctttaaaagaaattaaatttattcttgtttacag

SEQ ID NO: 6 - DNA, 639 bp, Rattus
gttcaagagtggattgttatcgcttatatttttacctatgctattgaaaaaattcgtgaggtctttatgtctgaagctgggaaaatcagccagaagatcaaagtgtggtttagtgactacttcaatgtcagtgacacaattgccataatttctttcttcgttggatttggactaagatttggagcaaaatggaactatattaatgcatatgataatcatgtttttgttgctggaagattaatttactgtcttaatataatattttggtatgtgcgcttgctagattttctagctgtaaatcaacaggcaggaccttatgtaatgatgattggaaaaatggtggccaatatgttctacattgtagtgataatggctcttgtattacttagttttggtgttcccagaaaagcaatactttatccacatgaagaaccgtcttggtctcttgctaaagatatagtttttcatccatactggatgatttttggtgaagtttatgcgtatgaaattgatgtgtgtgcaaatgattccgctctcccgacaatctgtggtcctggaacttggttgactccatttcttcaagcagtctacctctttgtacagtatatcattatggttaatcttcttattgcatttttc

SEQ ID NO: 7 - DNA, 657 bp, Rattus
agcatgtcttcttggtctcagctaggcctctgtgccaaaattgagtttttaagtaaagaggaaatgggaggcggtttacgaagagcagtcaaagtgctgtgtacctggtcagagcacgatgtcctgagatcagggcacctttatatcattaagtcgtttcttccagaggtgataaacacatggtcaagcatttataaagaagacacagttctgcatctctgtctcagagaaatacaacaacagagagcagcacagaagctcacatttgcctttaatcagatgaaacccaaatccataccatattctccaaggttcctggaagttttcctgctatactgccattcagcagggcagtggtttgctgtagaagaatgcatgactggggaatttaggaaatacaacaataataatggtgatgaaatcattccaaccaacactctggaagagatcatgctagccttcagccactggacctatgagtacaccagaggggagttgctggtacttgacttacaaggtgtgggagaaaacttgactgacccatctgtgataaaagctgaagagaaaagatcctgcgacatggtttttggccctgccaatttaggagaagatgcaataaaaaacttcagagccaaacatcactgtaattcttgctgt

SEQ ID NO: 8 - DNA, 234 bp, Rattus
gggcgcgggcgcgtccctgtggccagtcacccggcggagctggtcgcacaattatgaaagactcgacttctgctgctagcgctggagctgagttagttctgagaaggtttcccggggctgtccttgttcggtggcccgtgccaccgcctccggagacgctttccgatagatggctgcaggccgcggaggaggaggaggagccgctgcccttccggagtccgccccttgaggaga

SEQ ID NO: 9 - DNA, 1277 bp, Rattus
tactgaaccattgatctttgcctacatttcacaaaagtgtaactgacgtttttcctttcaggggggaattgatggtataggaaggtgtgtgcaaaatgagcttgctggccccacacatagttcagaggtaatgttctcattgaaaagcacctcaaggctacagatgaccgctgggaaatgttaacagtgacagaaagttagtgctctcggcttgtgaaggacctgaagctttgctgttgagggtggtgattctctgtcacttggtgcagtaccattaaccaaagtcaagtgtcttcagattttattggctgctcagttacagccattcagctaaagaatctaaattatgcagcttttcggttaagtggctgaagtcttcctcagtttgtgctctctgataatgatgttagctctcaactagatatttgtggtcacaggaaaactattccttacaattttgcttcacaggcatgttacaaagcctgcactaaaaaccatttgtcttccctccctccctctctttttcctgtagtgtagaagatcaaatccagggcctcatgaagaccattttctaagaaacattttaagaatcaactttagagtctttgtttatggatacagccagtttttgttaaacaaaactgaattgtgcaaaagggtttttttttttaacatttatcaatgttaagtaaaagaaagccatgataaataaggattaactcactgttcagtgggtgttccctgtgaggaaggttgcagttgtaacagcctgcagctgcatacatccccaaagatttacagacttagtgtatcaaatcagagtgtcatgtgaactctcacatttaaaattctataggaatgtgtcaatgtgaattctatgtctggtacttatttaagaaatcagttgttggattatccttatatagtatagggagatcacaatacaactttatgccaataaaataacttaattgcccagatatttttgcatatttagcaacaagaaaagcttatcatttgactcaagttttatgctttctctttcttttcatttcctaggtactaattttaatttttatttggaagggagcagtgtaaagcttatttgtattcaatagtgtatctcatagatacagacaaggccgcagagataagctgttaaatagtgtttaaagttgatgtggagagaaaaggtgtattacttaaagatactataccatatatgttttgtatatcattaaatctttaaaagaaattaaatttattcttgtttacag

SEQ ID NO: 10 - DNA, 23 bp, Artificial Sequence
  Oligonucleotide primer
agctggtcgcacaattatgaaag

SEQ ID NO: 11 - DNA, 22 bp, Artificial Sequence
  Oligonucleotide primer
atactctgcgacagcctcatca

SEQ ID NO: 12 - DNA, 23 bp, Artificial Sequence
  Oligonucleotide primer
tgctgttgtctgatatgtggatg

SEQ ID NO: 13 - DNA, 24 bp, Artificial Sequence
  Oligonucleotide primer
caccttttctctccacatcaactt

SEQ ID NO: 14 - DNA, 5592 bp, Mus musculus
atgtcccagaaatcctggatagagagcactttgaccaagagggagtgtgtatatattataccaagctccaaagaccctcacagatgtcttccaggatgtcagatttgtcagcaacttgtcagatgtttctgtggtcgtttggtcaagcaacatgcatgctttactgcaagtcttgccatgaaatactcagatgtgagattgggtgaacactttaaccaggcaatagaagaatggtctgtggaaaagcacacggagcagagcccaacagatgcttatggagtcatcaattttcaagggggttctcattcctacagagctaagtatgtgagactatcatatgataccaaacctgaaatcattctgcaacttctgcttaaagaatggcaaatggagttacccaaacttgttatttctgtacatggaggcatgcagaagtttgaacttcatccaagaatcaagcagttgcttggaaagggtcttattaaagctgcagttacaaccggagcttggattttaactggaggagtcaatacaggtgtggcaaaacatgttggtgatgccctcaaagaacatgcttccagatcatctcgaaaaatttgcactattggaatagctccatggggagtgatagaaaacagaaatgatcttgttgggagagatgtggttgctccttatcaaaccctattgaatcccttgagcaaattgaatgttctgaataatctacactcccatttcatcttggtggatgatggcactgttggaaagtatggggcagaagtcagactgagaagagaacttgaaaaaaccattaatcagcaaagaattcatgctagaattgggcaaggagttcctgtggtggctttgatatttgaaggcgggccaaatgtcatccttacagtactggagtaccttcaggaaagccccccagttccagttgttgtgtgtgaagggacaggcagagctgcagatttactagcctatatccacaaacagacagaggaaggaggaaatcttcctgatgcagcagagcctgatattatatcaactatcaagaaaacatttaactttggccagagtgaagcagttcatttatttcaaacaatgatggagtgtatgaaaaaaaaagagcttatcactgtttttcacattggatcagaggatcatcaagatatagatgtggccatactcactgcactgctgaaaggtactaatgcatctgcatttgaccagcttatccttacactggcatgggacagagttgatattgccaaaaatcatgtatttgtttatggacaacagtggctggttggatccttggaacaggctatgcttgatgctcttgtaatggacagagtttcatttgtaaaacttcttattgaaaacggagtaagcatgcataaattccttaccattcccagactggaagaactttataacactaaacaaggtccaaccaatccaatgttgttccatctcattcgggatgtcaagcagggtaatctccccccggggtacaagatcactttaattgatataggacttgtgattgagtatctcatgggaggaacctacagatgcacatacacacgaaaacgttttcgattgatatataatagtcttggtggaaataaccggaggtcaggtcgaaatacctccagcagcacccctcagttgcgaaagagtcatgaaacttttggcaatagagctgataaaaaggaaaaaatgagacacaatcatttcattaaaacagcccaaccctacagaccaaagatggatgcatctatggaagaaggaaagaagaaaagaaccaaagatgaaattgtagatatagatgatccagagaccaagcgctttccttatcctcttaatgaattattaatttgggcttgccttatgaagaggcaggtcatggcccgctttttatggcagcatggtgaagaatcaatggctaaagcattagttgcctgtaaaatctatcgttcaatggcttatgaggcaaagcagagtgacctggtagatgatacttcagaggaactgaagcagtattccaatgattttggccaactggcagttgaattactggaacagtccttcagacaggatgaaacgatggctatgaaattactcacttatgaactcaaaaactggagtaattcaacctgcctcaagttagcagtttcttcaagacttagaccttttgtagctcacacttgtacacagatgttgttatctgatatgtggatgggacggctgaatatgagaaaaaattcctggtataaggtcatattaagcattttagttccacctgccatattaatgctagagtataaaaccaaggctgaaatgtcccatatcccacaatctcaagatgctcatcaaatgacgatggaggatagtgaaaacaattttcacaacataacagaagagatacccatggaagtatttaaagaagtaaagattttggacagcagtgatggaaagaatgaaatggagatacatattaaatcaaaaaagcttccaatcacacgaaaattttatgccttttatcatgcaccaattgtaaagttctggtttaacacattggcatatttaggatttctgatgctttatacatttgtagttcttgtaaaaatggaacagttaccttcagttcaagaatggattgttatcgcttatatttttacctatgctattgaaaaagtccgtgaggtcttcatgtctgaagctgggaaaatcagccagaagattaaagtatggtttagtgactacttcaatgtcagtgacacaattgccatcatttctttctttgttggatttggactaagatttggagcaaaatggaactatattaatgcatatgataatcatgtttttgtggctggaagattaatttactgtcttaatataatattttggtatgtgcgtttgctagactttctagccgtaaatcaacaggcaggaccttatgtaatgatgattggaaaaatggtggccaatatgttctacattgtagtgataatggctcttgtattgcttagttttggtgttcccagaaaagcaatactttatccacatgaagaaccatcttggtctcttgctaaagatatagtttttcatccatactggatgatttttggtgaagtttatgcatatgaaattgatgtgtgtgcaaatgactccactctcccgacaatctgtggtcctggaacttggttgactccatttcttcaagcagtctacctctttgtacagtatatcattatggttaatctccttatcgcatttttcaataatgtatatttacaagtgaaggcaatttccaatattgtatggaagtatcagcggtatcattttattatggcttatcatgaaaaaccagtcctgcctcctcctcttatcatcctcagccatatagtttcactgttttgctgtgtatgcaaaagaagaaagaaagataagacttccgatgggccaaaacttttcttaacagaagaagatcaaaagaaactccatgattttgaagagcagtgtgttgagatgtactttgatgagaaagatgacaaattcaattctgggagtgaagagagaatccgggtcacttttgaaagagtggagcagatgagcattcagattaaagaagttggagatcgtgtcaactacataaaaagatcattacagtctttagattctcaaattggtcatctgcaagatctctcagccctaacagtagatacattgaaaacacttacagcccagaaagcttcagaagctagtaaagtgcacaatgagatcacacgagaattgagtatttccaaacacttggctcagaatcttattgatgatgttcctgtaagacctttgtggaagaaacctagtgctgtaaacacactgagttcctctcttcctcaaggtgatcgggaaagtaataatccttttctttgtaatatttttatgaaagatgaaaaagacccccaatataatctgtttggacaagatttgcccgtgataccccagagaaaagaattcaacattccagaggctggttcctcctgtggtgccttattcccaagtgctgtttctcccccagaattacgacagagacgacatggggtagaaatgttaaaaatatttaataaaaatcaaaaattaggcagttcacctaatagttcaccacatatgtcctccccaccaaccaaattttctgtgagtaccccatcccagccaagttgcaaaagccacttggaatccacaaccaaagatcaagaacccattttctataaagctgcagaaggggataacatagaatttggagcatttgtgggacacagagatagtatggacttacagaggtttaaagaaacatcaaacaaaataagagaactgttatctaatgatactcctgaaaacactctgaaacatgtgggtgctgctggatatagtgaatgttgtaagacttctacttctcttcactcagtgcaagcagaaagctgtagtagaagagcgtcgacggaagactctccagaagtcgattctaaagcagctttgttaccggattggttacgagatagaccatcaaacagagaaatgccatctgaaggaggaacattaaatggtcttgcttctccatttaagcccgttttggatacaaattactattattcagctgtggaaagaaataacctgatgaggttgtcacagagtattcccttcgttcctgtacctccacgaggcgagcctgtcacagtgtaccgtctggaggagagttctcccagtatactgaataacagcatgtcttcatggtctcagctaggcctctgtgccaaaattgagtttttaagtaaagaggaaatgggaggtggtttacgaagagcagtcaaagtgctgtgtacctggtcagagcacgatatcctgaagtcagggcatctctatatcattaagtcatttcttcctgaggtgataaacacatggtcaagcatttataaagaagatacggttctacatctctgtctcagagaaatacaacaacagagagcagcacaaaagctcacatttgcctttaatcagatgaaacccaaatccataccatattctccaaggttccttgaagttttcctgttgtactgccattcagcagggcagtggtttgctgtagaagagtgcatgactggtgaatttagaaaatacaacaacaataatggtgatgaaatcattcctacaaatactctagaagagatcatgctagcctttagccactggacctatgaatataccagaggggagttactggtacttgacttacaaggagtgggagaaaacttgactgacccatctgtaataaaagctgaagaaaaaagatcctgtgacatggtttttggccctgccaatctaggagaagatgcaataaaaaacttcagagccaaacatcactgtaattcttgctgtcgaaagcttaaacttccagatttgaagaggaatgactacacgcctgataaaattatatttcctcaggatgagtcatcagatttgaatcttcaatctggaaattccaccaaagaatcagaagcaacaaattctgttcgtctgatgttatag

SEQ ID NO: 15 - DNA, 5534 bp, Homo sapiens
atgtcccagaaatcctggatagaaagcactttgaccaagagggaatgtgtatatattataccaagttccaaggaccctcacagatgccttccaggatgtcaaatttgtcagcaactcgtcaggtgtttttgtggtcgcttggtcaagcaacatgcttgttttactgcaagtcttgccatgaaatactcagatgtgaaattgggtgaccattttaatcaggcaatagaagaatggtctgtggaaaagcatacagaacagagcccaacggatgcttatggagtcataaattttcaagggggttctcattcctacagagctaagtatgtgaggctatcatatgacaccaaacctgaagtcattctgcaacttctgcttaaagaatggcaaatggagttacccaaacttgttatctctgtacatgggggcatgcagaaatttgagcttcacccacgaatcaagcagttgcttggaaaaggtcttattaaagctgcagttacaactggagcctggattttaactggaggagtaaacacaggtgtggcaaaacatgttggagatgccctcaaagaacatgcttccagatcatctcgaaagatttgcactatcggaatagctccatggggagtgattgaaaacagaaatgatcttgttgggagagatgtggttgctccttatcaaaccttattgaaccccctgagcaaattgaatgttttgaataatctgcattcccatttcatattggtggatgatggcactgttggaaagtatggggcggaagtcagactgagaagagaacttgaaaaaactattaatcagcaaagaattcatgctaggattggccagggtgtccctgtggtggcacttatatttgagggtgggccaaatgttatcctcacagttcttgaataccttcaggaaagcccccctgttccagtagttgtgtgtgaaggaacaggcagagctgcagatctgctagcgtatattcataaacaaacagaagaaggagggaatcttcctgatgcagcagagcccgatattatttccactatcaaaaaaacatttaactttggccagaatgaagcacttcatttatttcaaacactgatggagtgcatgaaaagaaaggagcttatcactgttttccatattgggtcagatgaacatcaagatatagatgtagcaatacttactgcactgctaaaaggtactaatgcatctgcatttgaccagcttatccttacattggcatgggatagagttgacattgccaaaaatcatgtatttgtttatggacagcagtggctggttggatccttggaacaagctatgcttgatgctcttgtaatggatagagttgcatttgtaaaacttcttattgaaaatggagtaagcatgcataaattccttaccattccgagactggaagaactttacaacactaaacaaggtccaactaatccaatgctgtttcatcttgttcgagacgtcaaacagggaaatcttcctccaggatataagatcactctgattgatataggacttgttattgaatatctcatgggaggaacctacagatgcacctatactaggaaacgttttcgattaatatataatagtcttggtggaaataatcggaggtctggccgaaatacctccagcagcactcctcagttgcgaaagagtcatgaatcttttggcaatagggcagataaaaaggaaaaaatgaggcataaccatttcattaagacagcacagccctaccgaccaaagattgatacagttatggaagaaggaaagaagaaaagaaccaaagatgaaattgtagacattgatgatccagaaaccaagcgctttccttatccacttaatgaacttttaatttgggcttgccttatgaagaggcaggtcatggcccgttttttatggcaacatggtgaagaatcaatggctaaagcattagttgcctgtaagatctatcgttcaatggcatatgaagcaaagcagagtgacctggtagatgatacttcagaagaactaaaacagtattccaatgattttggtcagttggccgttgaattattagaacagtccttcagacaagatgaaaccatggctatgaaattgctcacttatgaactgaagaactggagtaattcaacctgccttaagttagcagtttcttcaagacttagaccttttgtagctcacacctgtacacaaatgttgttatctgatatgtggatgggaaggctgaatatgaggaaaaattcctggtacaaggtcatactaagcattttagttccacctgccatattgctgttagagtataaaactaaggctgaaatgtcccatatcccacaatctcaagatgctcatcagatgacaatggatgacagcgaaaacaactttcagaacataacagaagagatccccatggaagtgtttaaagaagtacggattttggatagtaatgaaggaaagaatgagatggagatacaaatgaaatcaaaaaagcttccaattacgcgaaagttttatgccttttatcatgcaccaattgtaaaattctggtttaacacgttggcatatttaggatttctgatgctttatacatttgtggttcttgtacaaatggaacagttaccttcagttcaagaatggattgttattgcttatatttttacttatgccattgagaaaagtgattacttcaacatcagtgatacaattgccataatttctttcttcattggatttggactaagatttggagcaaaatggaactttgcaaatgcatatgataatcatgtttttgtggctggaagattaatttactgtcttaacataatattttggtatgtgcgtttgctagattttctagctgtaaatcaacaggcaggaccttatgtaatgatgattggaaaaatggtggccaatatgttctacattgtagtgattatggctcttgtattacttagttttggtgttcccagaaaggcaatactttatcctcatgaagcaccatcttggactcttgctaaagatatagtttttcacccatactggatgatttttggtgaagtttatgcatacgaaattgatgtgtgtgcaaatgattctgttatccctcaaatctgtggtcctgggacgtggttgactccatttcttcaagcagtctacctctttgtacagtatatcattatggttaatcttcttattgcatttttcaacaatgtgtatttacaagtgaaggcaatttccaatattgtatggaagtaccagcgttatcattttattatggcttatcatgagaaaccagttctgcctcctccacttatcattcttagccatatagtttctctgttttgctgcatatgtaagagaagaaagaaagataagacttccgatggaccaaaacttttcttaacagaagaagatcaaaagaaacttcatgattttgaagagcagtgtgttgaaatgtatttcaatgaaaaagatgacaaatttcattctgggagtgaagagagaattcgtgtcacttttgaaagagtggaacagatgtgcattcagattaaagaagttggagatcgtgtcaactacataaaaagatcattacaatcattagattctcaaattggccatttgcaagatctttcagccctgacggtagatacattaaaaacactcactgcccagaaagcgtcggaagctagcaaagttcataatgaaatcacacgagaactgagcatttccaaacacttggctcaaaaccttattgatgatggtcctgtaagaccttctgtatggaaaaagcatggtgttgtaaatacacttagctcctctcttcctcaaggtgatcttgaaagtaataatccttttcattgtaatattttaatgaaagatgacaaagatccccagtgtaatatatttggtcaagacttacctgcagtaccccagagaaaagaatttaattttccagaggctggttcctcttctggtgccttattcccaagtgctgtttcccctccagaactgcgacagagactacatggggtagaactcttaaaaatatttaataaaaatcaaaaattaggcagttcatctactagcataccacatctgtcatccccaccaaccaaattttttgttagtacaccatctcagccaagttgcaaaagccacttggaaactggaaccaaagatcaagaaactgtttgctctaaagctacagaaggagataatacagaatttggagcatttgtaggacacagagatagcatggatttacagaggtttaaagaaacatcaaacaagataaaaatactatccaataacaatacttctgaaaacactttgaaacgagtgagttctcttgctggatttactgactgtcacagaacttccattcctgttcattcaaaacaagaaaaaatcagtagaaggccatctaccgaagacactcatgaagtagattccaaagcagctttaataccggtttggttacaagatagaccatcaaacagagaaatgccatctgaagaaggaacattaaatggtctcacttctccatttaagccagctatggatacaaattactattattcagctgtggaaagaaataacttgatgaggttatcacagagcattccatttacacctgtgcctccaagaggggagcctgtcacagtgtatcgtttggaagagagttcacccaacatactaaataacagcatgtcttcttggtcacaactaggcctctgtgccaaaatagagtttttaagcaaagaggagatgggaggaggtttacgaagagctgtcaaagtacagtgtacctggtcagaacatgatatcctcaaatcagggcatctttatattatcaaatcttttcttccagaggtggttaatacatggtcaagtatttacaaagaagatacagttctgcatctctgtctgagagaaattcaacaacagagagcagcacaaaagcttacgtttgcctttaatcaaatgaaacccaaatccataccatattctccaaggttccttgaagttttcctgctgtattgccattcagcaggacagtggtttgctgtggaagaatgtatgactggagaatttagaaaatacaacaataataatggagatgagattattccaactaatactctggaagagatcatgctagcctttagccactggacttacgaatatacaagaggggagttactggtacttgatttgcaaggtgttggtgaaaatttgactgacccatctgtgataaaagcagaagaaaagagatcctgtgatatggtttttggcccagcaaatctaggagaagatgcaattaaaaacttcagagcaaaacatcactgtaattcttgctgtagaaagcttaaacttccagatctgaagaggaatgattatacgcctgataaaattatatttcctcaggatgagccttcagatttgaatcttcagcctggaaattccaccaaagaatcagaatcaactaattctgttcgtctgatgttata

SEQ ID NO: 16 - protein, 1863 aa, Mus musculus
MSQKSWIESTLTKRECVYIIPSSKDPHRCLPGCQICQQLVRCFCGRLVKQHACFTASLAMKYSDVRLGEHFNQAIEEWSVEKHTEQSPTDAYGVINFQGGSHSYRAKYVRLSYDTKPEIILQLLLKEWQMELPKLVISVHGGMQKFELHPRIKQLLGKGLIKAAVTTGAWILTGGVNTGVAKHVGDALKEHASRSSRKICTIGIAPWGVIENRNDLVGRDVVAPYQTLLNPLSKLNVLNNLHSHFILVDDGTVGKYGAEVRLRRELEKTINQQRIHARIGQGVPVVALIFEGGPNVILTVLEYLQESPPVPVVVCEGTGRAADLLAYIHKQTEEGGNLPDAAEPDIISTIKKTFNFGQSEAVHLFQTMMECMKKKELITVFHIGSEDHQDIDVAILTALLKGTNASAFDQLILTLAWDRVDIAKNHVFVYGQQWLVGSLEQAMLDALVMDRVSFVKLLIENGVSMHKFLTIPRLEELYNTKQGPTNPMLFHLIRDVKQGNLPPGYKITLIDIGLVIEYLMGGTYRCTYTRKRFRLIYNSLGGNNRRSGRNTSSSTPQLRKSHETFGNRADKKEKMRHNHFIKTAQPYRPKMDASMEEGKKKRTKDEIVDIDDPETKRFPYPLNELLIWACLMKRQVMARFLWQHGEESMAKALVACKIYRSMAYEAKQSDLVDDTSEELKQYSNDFGQLAVELLEQSFRQDETMAMKLLTYELKNWSNSTCLKLAVSSRLRPFVAHTCTQMLLSDMWMGRLNMRKNSWYKVILSILVPPAILMLEYKTKAEMSHIPQSQDAHQMTMEDSENNFHNITEEIPMEVFKEVKILDSSDGKNEMEIHIKSKKLPITRKFYAFYHAPIVKFWFNTLAYLGFLMLYTFVVLVKMEQLPSVQEWIVIAYIFTYAIEKVREVFMSEAGKISQKIKVWFSDYFNVSDTIAIISFFVGFGLRFGAKWNYINAYDNHVFVAGRLIYCLNIIFWYVRLLDFLAVNQQAGPYVMMIGKMVANMFYIVVIMALVLLSFGVPRKAILYPHEEPSWSLAKDIVFHPYWMIFGEVYAYEIDVCANDSTLPTICGPGTWLTPFLQAVYLFVQYIIMVNLLIAFFNNVYLQVKAISNIVWKYQRYHFIMAYHEKPVLPPPLIILSHIVSLFCCVCKRRKKDKTSDGPKLFLTEEDQKKLHDFEEQCVEMYFDEKDDKFNSGSEERIRVTFERVEQMSIQIKEVGDRVNYIKRSLQSLDSQIGHLQDLSALTVDTLKTLTAQKASEASKVHNEITRELSISKHLAQNLIDDVPVRPLWKKPSAVNTLSSSLPQGDRESNNPFLCNIFMKDEKDPQYNLFGQDLPVIPQRKEFNIPEAGSSCGALFPSAVSPPELRQRRHGVEMLKIFNKNQKLGSSPNSSPHMSSPPTKFSVSTPSQPSCKSHLESTTKDQEPIFYKAAEGDNIEFGAFVGHRDSMDLQRFKETSNKIRELLSNDTPENTLKHVGAAGYSECCKTSTSLHSVQAESCSRRASTEDSPEVDSKAALLPDWLRDRPSNREMPSEGGTLNGLASPFKPVLDTNYYYSAVERNNLMRLSQSIPFVPVPPRGEPVTVYRLEESSPSILNNSMSSWSQLGLCAKIEFLSKEEMGGGLRRAVKVLCTWSEHDILKSGHLYIIKSFLPEVINTWSSIYKEDTVLHLCLREIQQQRAAQKLTFAFNQMKPKSIPYSPRFLEVFLLYCHSAGQWFAVEECMTGEFRKYNNNNGDEIIPTNTLEEIMLAFSHWTYEYTRGELLVLDLQGVGENLTDPSVIKAEEKRSCDMVFGPANLGEDAIKNFRAKHHCNSCCRKLKLPDLKRNDYTPDKIIFPQDESSDLNLQSGNSTKESEATNSVRLML

SEQ ID NO: 17 - protein, 1863 aa, Artificial Sequence
  Consensus sequence between rat and mouse
  MISC_FEATURE (60)..(60): Xaa = non-conserved amino acid
  MISC_FEATURE (66)..(66): Xaa = conserved amino acid
  MISC_FEATURE (483)..(483): Xaa = non-conserved amino acid
  MISC_FEATURE (484)..(484): Xaa = conserved amino acid
  MISC_FEATURE (550)..(550): Xaa = non-conserved amino acid
  MISC_FEATURE (551)..(551): Xaa = conserved amino acid
  MISC_FEATURE (825)..(825): Xaa = conserved amino acid
  MISC_FEATURE (877)..(877): Xaa = conserved amino acid
  MISC_FEATURE (901)..(901): Xaa = conserved amino acid
  MISC_FEATURE (1057)..(1057): Xaa = non-conserved amino acid
  MISC_FEATURE (1061)..(1061): Xaa = conserved amino acid
  MISC_FEATURE (1145)..(1145): Xaa = conserved amino acid
  MISC_FEATURE (1287)..(1287): Xaa = non-conserved amino acid
  MISC_FEATURE (1293)..(1293): Xaa = conserved amino acid
  MISC_FEATURE (1415)..(1415): Xaa = non-conserved amino acid
  MISC_FEATURE (1421)..(1421): Xaa = conserved amino acid
  MISC_FEATURE (1476)..(1476): Xaa = non-conserved amino acid
  MISC_FEATURE (1479)..(1479): Xaa = non-conserved amino acid
  MISC_FEATURE (1480)..(1480): Xaa = conserved amino acid
  MISC_FEATURE (1483)..(1483): Xaa = conserved amino acid
  MISC_FEATURE (1485)..(1485): Xaa = non-conserved amino acid
  MISC_FEATURE (1486)..(1486): Xaa = conserved amino acid
  MISC_FEATURE (1487)..(1487): Xaa = non-conserved amino acid
  MISC_FEATURE (1492)..(1492): Xaa = conserved amino acid
  MISC_FEATURE (1494)..(1494): Xaa = conserved amino acid
  MISC_FEATURE (1508)..(1508): Xaa = conserved amino acid
  MISC_FEATURE (1512)..(1512): Xaa = conserved amino acid
  MISC_FEATURE (1525)..(1525): Xaa = conserved amino acid
  MISC_FEATURE (1636)..(1636): Xaa = conserved amino acid
  MISC_FEATURE (1638)..(1638): Xaa = conserved amino acid
  MISC_FEATURE (1846)..(1846): Xaa = conserved amino acid
MSQKSWIESTLTKRECVYIIPSSKDPHRCLPGCQICQQLVRCFCGRLVKQHACFTASLAXKYSDVXLGEHFNQAIEEWSVEKHTEQSPTDAYGVINFQGGSHSYRAKYVRLSYDTKPEIILQLLLKEWQMELPKLVISVHGGMQKFELHPRIKQLLGKGLIKAAVTTGAWILTGGVNTGVAKHVGDALKEHASRSSRKICTIGIAPWGVIENRNDLVGRDVVAPYQTLLNPLSKLNVLNNLHSHFILVDDGTVGKYGAEVRLRRELEKTINQQRIHARIGQGVPVVALIFEGGPNVILTVLEYLQESPPVPVVVCEGTGRAADLLAYIHKQTEEGGNLPDAAEPDIISTIKKTFNFGQSEAVHLFQTMMECMKKKELITVFHIGSEDHQDIDVAILTALLKGTNASAFDQLILTLAWDRVDIAKNHVFVYGQQWLVGSLEQAMLDALVMDRVSFVKLLIENGVSMHKFLTIPRLEELYNTKQGXTNPMLFHLIRDVKQGNLPPGYKITLIDIGLVIEYLMGGTYRCTYTRKRFRLIYNSLGGNNRRSGRNXSSSTPQLRKSHETFGNRADKKEKMRHNHFIKTAQPYRPKMDASMEEGKKKRTKDEIVDIDDPETKRFPYPLNELLIWACLMKRQVMARFLWQHGEESMAKALVACKIYRSMAYEAKQSDLVDDTSEELKQYSNDFGQLAVELLEQSFRQDETMAMKLLTYELKNWSNSTCLKLAVSSRLRPFVAHTCTQMLLSDMWMGRLNMRKNSWYKVILSILVPPAILMLEYKTKAEMSHIPQSQDAHQMTMEDSENNFHNITEEIPMEVFKEVKILDSSXGKNEMEIHIKSKKLPITRKFYAFYHAPIVKFWFNTLAYLGFLMLYTFVVLVXMEQLPSVQEWIVIAYIFTYAIEKXREVFMSEAGKISQKIKVWFSDYFNVSDTIAIISFFVGFGLRFGAKWNYINAYDNHVFVAGRLIYCLNIIFWYVRLLDFLAVNQQAGPYVMMIGKMVANMFYIVVIMALVLLSFGVPRKAILYPHEEPSWSLAKDIVFHPYWMIFGEVYAYEIDVCANDSXLPTICGPGTWLTPFLQAVYLFVQYIIMVNLLIAFFNNVYLQVKAISNIVWKYQRYHFIMAYHEKPVLPPPLIILSHIVSLFCCXCKRRKKDKTSDGPKLFLTEEDQKKLHDFEEQCVEMYFDEKDDKFNSGSEERIRVTFERVEQMSIQIKEVGDRVNYIKRSLQSLDSQIGHLQDLSALTVDTLKTLTAQKASEASKVHNEITRELSISKHLAQNLIDDVPVRPXWKKPSXVNTLSSSLPQGDRESNNPFLCNIFMKDEKDPQYNLFGQDLPVIPQRKEFNIPEAGSSCGALFPSAVSPPELRQRRHGVEMLKIFNKNQKLGSSPNSSPHMSSPPTKFSVSTPSQPSCKSHLESTTKDXEPIFYKAAEGDNIEFGAFVGHRDSMDLQRFKETSNKIRELLSNDTPENTLKHVGAAGYXECXKTXTSLHSXQXESCSRRASTEDSPXVDSXAALLPDWLRDRPXNREMPSEGGTLNGLASPFKPVLDTNYYYSAVERNNLMRLSQSIPFVPVPPRGEPVTVYRLEESSPSILNNSMSSWSQLGLCAKIEFLSKEEMGGGLRRAVKVLCTWSEHDXLXSGHLYIIKSFLPEVINTWSSIYKEDTVLHLCLREIQQQRAAQKLTFAFNQMKPKSIPYSPRFLEVFLLYCHSAGQWFAVEECMTGEFRKYNNNNGDEIIPTNTLEEIMLAFSHWTYEYTRGELLVLDLQGVGENLTDPSVIKAEEKRSCDMVFGPANLGEDAIKNFRAKHHCNSCCRKLKLPDLKRNDYTPDKIIFPQDESSDLNLQXGNSTKESEATNSVRLML

SEQ ID NO: 18 - protein, 1864 aa, Homo sapiens
MSQKSWIESTLTKRECVYIIPSSKDPHRCLPGCQICQQLVRCFCGRLVKQHACFTASLAMKYSDVKLGDHFNQAIEEWSVEKHTEQSPTDAYGVINFQGGSHSYRAKYVRLSYDTKPEVILQLLLKEWQMELPKLVISVHGGMQKFELHPRIKQLLGKGLIKAAVTTGAWILTGGVNTGVAKHVGDALKEHASRSSRKICTIGIAPWGVIENRNDLVGRDVVAPYQTLLNPLSKLNVLNNLHSHFILVDDGTVGKYGAEVRLRRELEKTINQQRIHARIGQGVPVVALIFEGGPNVILTVLEYLQESPPVPVVVCEGTGRAADLLAYIHKQTEEGGNLPDAAEPDIISTIKKTFNFGQNEALHLFQTLMECMKRKELITVFHIGSDEHQDIDVAILTALLKGTNASAFDQLILTLAWDRVDIAKNHVFVYGQQWLVGSLEQAMLDALVMDRVAFVKLLIENGVSMHKFLTIPRLEELYNTKQGPTNPMLFHLVRDVKQGNLPPGYKITLIDIGLVIEYLMGGTYRCTYTRKRFRLIYNSLGGNNRRSGRNTSSSTPQLRKSHESFGNRADKKEKMRHNHFIKTAQPYRPKIDTVMEEGKKKRTKDEIVDIDDPETKRFPYPLNELLIWACLMKRQVMARFLWQHGEESMAKALVACKIYRSMAYEAKQSDLVDDTSEELKQYSNDFGQLAVELLEQSFRQDETMAMKLLTYELKNWSNSTCLKLAVSSRLRPFVAHTCTQMLLSDMWMGRLNMRKNSWYKVILSILVPPAILLLEYKTKAEMSHIPQSQDAHQMTMDDSENNFQNITEEIPMEVFKEVRILDSNEGKNEMEIQMKSKKLPITRKFYAFYHAPIVKFWFNTLAYLGFLMLYTFVVLVQMEQLPSVQEWIVIAYIFTYAIEKVREIFMSEAGKVNQKIKVWFSDYFNISDTIAIISFFIGFGLRFGAKWNFANAYDNHVFVAGRLIYCLNIIFWYVRLLDFLAVNQQAGPYVMMIGKMVANMFYIVVIMALVLLSFGVPRKAILYPHEAPSWTLAKDIVFHPYWMIFGEVYAYEIDVCANDSVIPQICGPGTWLTPFLQAVYLFVQYIIMVNLLIAFFNNVYLQVKAISNIVWKYQRYHFIMAYHEKPVLPPPLIILSHIVSLFCCICKRRKKDKTSDGPKLFLTEEDQKKLHDFEEQCVEMYFNEKDDKFHSGSEERIRVTFERVEQMCIQIKEVGDRVNYIKRSLQSLDSQIGHLQDLSALTVDTLKTLTAQKASEASKVHNEITRELSISKHLAQNLIDDGPVRPSVWKKHGVVNTLSSSLPQGDLESNNPFHCNILMKDDKDPQCNIFGQDLPAVPQRKEFNFPEAGSSSGALFPSAVSPPELRQRLHGVELLKIFNKNQKLGSSSTSIPHLSSPPTKFFVSTPSQPSCKSHLETGTKDQETVCSKATEGDNTEFGAFVGHRDSMDLQRFKETSNKIKILSNNNTSENTLKRVSSLAGFTDCHRTSIPVHSKQEKISRRPSTEDTHEVDSKAALIPVWLQDRPSNREMPSEEGTLNGLTSPFKPAMDTNYYYSAVERNNLMRLSQSIPFTPVPPRGEPVTVYRLEESSPNILNNSMSSWSQLGLCAKIEFLSKEEMGGGLRRAVKVQCTWSEHDILKSGHLYIIKSFLPEVVNTWSSIYKEDTVLHLCLREIQQQRAAQKLTFAFNQMKPKSIPYSPRFLEVFLLYCHSAGQWFAVEECMTGEFRKYNNNNGDEIIPTNTLEEIMLAFSHWTYEYTRGELLVLDLQGVGENLTDPSVIKAEEKRSCDMVFGPANLGEDAIKNFRAKHHCNSCCRKLKLPDLKRNDYTPDKIIFPQDEPSDLNLQPGNSTKESESTNSVRLML

SEQ ID NO: 19 - protein, 1864 aa, Artificial Sequence
  Consensus sequence between rat and human
  MISC_FEATURE (60)..(60): Xaa = non-conserved amino acid
  MISC_FEATURE (69)..(69): Xaa = conserved amino acid
  MISC_FEATURE (119)..(119): Xaa = conserved amino acid
  MISC_FEATURE (359)..(359): Xaa = conserved amino acid
  MISC_FEATURE (362)..(362): Xaa = conserved amino acid
  MISC_FEATURE (368)..(368): Xaa = conserved amino acid
  MISC_FEATURE (374)..(374): Xaa = conserved amino acid
  MISC_FEATURE (386)..(387): Xaa = conserved amino acid
  MISC_FEATURE (453)..(453): Xaa = conserved amino acid
  MISC_FEATURE (475)..(475): Xaa = non-conserved amino acid
  MISC_FEATURE (484)..(484): Xaa = conserved amino acid
  MISC_FEATURE (493)..(493): Xaa = conserved amino acid
  MISC_FEATURE (541)..(541): Xaa = non-conserved amino acid
  MISC_FEATURE (551)..(551): Xaa = conserved amino acid
  MISC_FEATURE (564)..(564): Xaa = conserved amino acid
  MISC_FEATURE (581)..(582): Xaa = non-conserved amino acid
  MISC_FEATURE (591)..(591): Xaa = conserved amino acid
  MISC_FEATURE (593)..(594): Xaa = conserved amino acid
  MISC_FEATURE (773)..(773): Xaa = conserved amino acid
  MISC_FEATURE (790)..(790): Xaa = non-conserved amino acid
  MISC_FEATURE (797)..(797): Xaa = conserved amino acid
  MISC_FEATURE (804)..(804): Xaa = conserved amino acid
  MISC_FEATURE (817)..(817): Xaa = non-conserved amino acid
  MISC_FEATURE (819)..(819): Xaa = conserved amino acid
  MISC_FEATURE (824)..(824): Xaa = conserved amino acid
  MISC_FEATURE (833)..(834): Xaa = conserved amino acid
  MISC_FEATURE (901)..(901): Xaa = conserved amino acid
  MISC_FEATURE (904)..(904): Xaa = conserved amino acid
  MISC_FEATURE (912)..(913): Xaa = conserved amino acid
  MISC_FEATURE (926)..(926): Xaa = non-conserved amino acid
  MISC_FEATURE (937)..(937): Xaa = conserved amino acid
  MISC_FEATURE (949)..(950): Xaa = conserved amino acid
  MISC_FEATURE (1003)..(1003): Xaa = non-conserved amino acid
  MISC_FEATURE (1027)..(1027): Xaa = conserved amino acid
  MISC_FEATURE (1031)..(1031): Xaa = conserved amino acid
  MISC_FEATURE (1036)..(1036): Xaa = non-conserved amino acid
  MISC_FEATURE (1038)..(1038): Xaa = non-conserved amino acid
  MISC_FEATURE (1061)..(1062): Xaa = conserved amino acid
  MISC_FEATURE (1064)..(1064): Xaa = conserved amino acid
  MISC_FEATURE (1180)..(1180): Xaa = non-conserved amino acid
  MISC_FEATURE (1183)..(1183): Xaa = conserved amino acid
  MISC_FEATURE (1190)..(1190): Xaa = conserved amino acid
  MISC_FEATURE (1208)..(1208): Xaa = conserved amino acid
  MISC_FEATURE (1254)..(1254): Xaa = non-conserved amino acid
  MISC_FEATURE (1259)..(1259): Xaa = non-conserved amino acid
  MISC_FEATURE (1263)..(1264): Xaa = non-conserved amino acid
  MISC_FEATURE (1278)..(1278): Xaa = non-conserved amino acid
  MISC_FEATURE (1282)..(1282): Xaa = conserved amino acid
  MISC_FEATURE (1285)..(1285): Xaa = non-conserved amino acid
  MISC_FEATURE (1287)..(1288): Xaa = conserved amino acid
  MISC_FEATURE (1289)..(1289): Xaa = non-conserved amino acid
  MISC_FEATURE (1292)..(1293): Xaa = conserved amino acid
  MISC_FEATURE (1297)..(1297): Xaa = non-conserved amino acid
  MISC_FEATURE (1305)..(1305): Xaa = non-conserved amino acid
  MISC_FEATURE (1307)..(1307): Xaa = conserved amino acid
  MISC_FEATURE (1313)..(1313): Xaa = non-conserved amino acid
  MISC_FEATURE (1314)..(1314): Xaa = conserved amino acid
  MISC_FEATURE (1318)..(1318): Xaa = conserved amino acid
  MISC_FEATURE (1320)..(1320): Xaa = non-conserved amino acid
  MISC_FEATURE (1322)..(1322): Xaa = conserved amino acid
  MISC_FEATURE (1327)..(1327): Xaa = conserved amino acid
  MISC_FEATURE (1329)..(1329): Xaa = conserved amino acid
  MISC_FEATURE (1336)..(1336): Xaa = conserved amino acid
  MISC_FEATURE (1337)..(1337): Xaa = non-conserved amino acid
  MISC_FEATURE (1345)..(1345): Xaa = conserved amino acid
  MISC_FEATURE (1352)..(1352): Xaa = conserved amino acid
  MISC_FEATURE (1355)..(1356): Xaa = non-conserved amino acid
  MISC_FEATURE (1358)..(1358): Xaa = non-conserved amino acid
  MISC_FEATURE (1368)..(1368): Xaa = non-conserved amino acid
  MISC_FEATURE (1369)..(1369): Xaa = conserved amino acid
  MISC_FEATURE (1374)..(1374): Xaa = conserved amino acid
  MISC_FEATURE (1383)..(1383): Xaa = non-conserved amino acid
  MISC_FEATURE (1387)..(1387): Xaa = non-conserved amino acid
  MISC_FEATURE (1388)..(1388): Xaa = conserved amino acid
  MISC_FEATURE (1389)..(1391): Xaa = non-conserved amino acid
  MISC_FEATURE (1394)..(1394): Xaa = non-conserved amino acid
  MISC_FEATURE (1399)..(1399): Xaa = non-conserved amino acid
  MISC_FEATURE (1402)..(1402): Xaa = conserved amino acid
  MISC_FEATURE (1417)..(1418): Xaa = conserved amino acid
  MISC_FEATURE (1422)..(1422): Xaa = conserved amino acid
  MISC_FEATURE (1424)..(1424): Xaa = non-conserved amino acid
  MISC_FEATURE (1425)..(1425): Xaa = conserved amino acid
  MISC_FEATURE (1426)..(1426): Xaa = non-conserved amino acid
  MISC_FEATURE (1427)..(1427): Xaa = conserved amino acid
  MISC_FEATURE (1429)..(1429): Xaa = non-conserved amino acid
  MISC_FEATURE (1430)..(1430): Xaa = conserved amino acid
  MISC_FEATURE (1435)..(1435): Xaa = non-conserved amino acid
  MISC_FEATURE (1437)..(1438): Xaa = non-conserved amino acid
  MISC_FEATURE (1441)..(1441): Xaa = non-conserved amino acid
  MISC_FEATURE (1445)..(1447): Xaa = non-conserved amino acid
  MISC_FEATURE (1452)..(1453): Xaa = non-conserved amino acid
  MISC_FEATURE (1457)..(1457): Xaa = non-conserved amino acid
  MISC_FEATURE (1460)..(1461): Xaa = conserved amino acid
  MISC_FEATURE (1462)..(1462): Xaa = non-conserved amino acid
  MISC_FEATURE (1463)..(1464): Xaa = conserved amino acid
  MISC_FEATURE (1466)..(1466): Xaa = conserved amino acid
  MISC_FEATURE (1468)..(1468): Xaa = conserved amino acid
  MISC_FEATURE (1470)..(1470): Xaa = non-conserved amino acid
  MISC_FEATURE (1474)..(1474): Xaa = conserved amino acid
  MISC_FEATURE (1476)..(1478): Xaa = conserved amino acid
  MISC_FEATURE (1481)..(1482): Xaa = conserved amino acid
  MISC_FEATURE (1483)..(1483): Xaa = non-conserved amino acid
  MISC_FEATURE (1486)..(1486): Xaa = conserved amino acid
  MISC_FEATURE (1488)..(1489): Xaa = conserved amino acid
  MISC_FEATURE (1490)..(1490): Xaa = non-conserved amino acid
  MISC_FEATURE (1491)..(1491): Xaa = conserved amino acid
  MISC_FEATURE (1494)..(1494): Xaa = conserved amino acid
  MISC_FEATURE (1496)..(1496): Xaa = non-conserved amino acid
  MISC_FEATURE (1497)..(1498): Xaa = conserved amino acid
  MISC_FEATURE (1502)..(1502): Xaa = conserved amino acid
  MISC_FEATURE (1507)..(1509): Xaa = conserved amino acid
  MISC_FEATURE (1513)..(1513): Xaa = conserved amino acid
  MISC_FEATURE (1517)..(1517): Xaa = conserved amino acid
  MISC_FEATURE (1519)..(1519): Xaa = conserved amino acid
  MISC_FEATURE (1520)..(1520): Xaa = non-conserved amino acid
  MISC_FEATURE (1522)..(1522): Xaa = conserved amino acid
  MISC_FEATURE (1526)..(1526): Xaa = conserved amino acid
  MISC_FEATURE (1534)..(1534): Xaa = conserved amino acid
  MISC_FEATURE (1541)..(1541): Xaa = conserved amino acid
  MISC_FEATURE (1547)..(1548): Xaa = conserved amino acid
  MISC_FEATURE (1572)..(1572): Xaa = conserved amino acid
  MISC_FEATURE (1576)..(1576): Xaa = non-conserved amino acid
  MISC_FEATURE (1592)..(1592): Xaa = conserved amino acid
  MISC_FEATURE (1629)..(1629): Xaa = conserved amino acid
  MISC_FEATURE (1637)..(1637): Xaa = conserved amino acid
  MISC_FEATURE (1639)..(1639): Xaa = conserved amino acid
  MISC_FEATURE (1654)..(1654): Xaa = conserved amino acid
  MISC_FEATURE (1784)..(1784): Xaa = non-conserved amino acid
  MISC_FEATURE (1791)..(1791): Xaa = non-conserved amino acid
  MISC_FEATURE (1840)..(1840): Xaa = conserved amino acid
  MISC_FEATURE (1847)..(1847): Xaa = conserved amino acid
  MISC_FEATURE (1856)..(1856): Xaa = conserved amino acid
MSQKSWIESTLTKRECVYIIPSSKDPHRCLPGCQICQQLVRCFCGRLVKQHACFTASLAXKYSDVKLGXHFNQAIEEWSVEKHTEQSPTDAYGVINFQGGSHSYRAKYVRLSYDTKPEXILQLLLKEWQMELPKLVISVHGGMQKFELHPRIKQLLGKGLIKAAVTTGAWILTGGVNTGVAKHVGDALKEHASRSSRKICTIGIAPWGVIENRNDLVGRDVVAPYQTLLNPLSKLNVLNNLHSHFILVDDGTVGKYGAEVRLRRELEKTINQQRIHARIGQGVPVVALIFEGGPNVILTVLEYLQESPPVPVVVCEGTGRAADLLAYIHKQTEEGGNLPDAAEPDIISTIKKTFNFGQXEAXHLFQTXMECMKXKELITVFHIGSXXHQDIDVAILTALLKGTNASAFDQLILTLAWDRVDIAKNHVFVYGQQWLVGSLEQAMLDALVMDRVXFVKLLIENGVSMHKFLTIPRLEELYNTKQGXTNPMLFHLXRDVKQGNLPPGYKITLIDIGLVIEYLMGGTYRCTYTRKRFRLIYNSLGGNNRRSGRNXSSSTPQLRKSHEXFGNRADKKEKMRHNHFIKTAQPYRPKXDXXMEEGKKKRTKDEIVDIDDPETKRFPYPLNELLIWACLMKRQVMARFLWQHGEESMAKALVACKIYRSMAYEAKQSDLVDDTSEELKQYSNDFGQLAVELLEQSFRQDETMAMKLLTYELKNWSNSTCLKLAVSSRLRPFVAHTCTQMLLSDMWMGRLNMRKNSWYKVILSILVPPAILXLEYKTKAEMSHIPQSQDAHQMTMXDSENNFXNITEEIPMEVFKEVXILDSXEGKNEMEIXXKSKKLPITRKFYAFYHAPIVKFWFNTLAYLGFLMLYTFVVLVQMEQLPSVQEWIVIAYIFTYAIEKXREXFMSEAGKXXQKIKVWFSDYFNXSDTIAIISFFXGFGLRFGAKWNXXNAYDNHVFVAGRLIYCLNIIFWYVRLLDFLAVNQQAGPYVMMIGKMVANMFYIVVIMALVLLSFGVPRKAILYPHEXPSWXLAKDIVFHPYWMIFGEVYAYEIDVCANDSXXPXICGPGTWLTPFLQAVYLFVQYIIMVNLLIAFFNNVYLQVKAISNIVWKYQRYHFIMAYHEKPVLPPPLIILSHIVSLFCCICKRRKKDKTSDGPKLFLTEEDQKKLHDFEEQCVEMYFXEKDDKFXSGSEERIRVTFERVEQMXIQIKEVGDRVNYIKRSLQSLDSQIGHLQDLSALTVDTLKTLTAQKASEASKVHNEITRELSISKHLAQNLIDDXPVRPXXWKKXXVVNTLSSSLPQGDXESNNPFXCNIXMKDXKDPQXNXFGQDLPXXPQRKEFNXPEAGSSXGALFPSAVSPPELRQRXHGVEXLKIFNKNQKLGSSXXSXPHXSSPPTKFXVSTPSQPSCKSHLEXXTKDXEXXXXKAXEGDNXEFGAFVGHRDSMDLQRFKETSNKIXXLXXNXTXENTLKXVXXXAGXXXCHXTXXXXHSXQEXXSRRXSTEDXXXVDSXAALXPXWLXDRPXNREMPSEXGTLNGLXSPFKPXXDTNYYYSAVERNNLMRLSQSIPFXPVPPRGEPVTVYRLEESSPXILNNSMSSWSQLGLCAKIEFLSKEEMGGGLRRAVKVXCTWSEHDXLXSGHLYIIKSFLPEVXNTWSSIYKEDTVLHLCLREIQQQRAAQKLTFAFNQMKPKSIPYSPRFLEVFLLYCHSAGQWFAVEECMTGEFRKYNNNNGDEIIPTNTLEEIMLAFSHWTYEYTRGELLVLDLQGVGENLTDPSVIKAEEKRSCDMVFGPANLGEDAIKNFRAKHHCNSCCRKLKLPDLKRNDYTPDKIIFPQDEXSDLNLQXGNSTKESEXTNSVRLML